(19) United States
(12) Patent Application Publication
(10) Pub. No.: US 2009/0209615 A1
(43) Pub. Date: Aug. 20, 2009

(54) INHIBITORS OF MATRIX METALLOPROTEINASES TO TREAT NEUROLOGICAL DISORDERS

(76) Inventors: Stuart Lipton, Rancho Santa Fe, CA (US); Alex Strongin, San Diego, CA (US); Shahriar Mobashery, Granger (IN); Zezong Gu, San Diego, CA (US)

Correspondence Address: SCHWEGMAN, LUNDBERG & WOESSNER, P.A., P.O. BOX 2938, MINNEAPOLIS, MN, 55402, US

(21) Appl. No.: 11/576,132

(22) PCT Filed: Sep. 27, 2005
(86) PCT No.: PCT/US05/34514

§ 371 Date: Jan. 22, 2008

Related U.S. Application Data
(60) Provisional application No. 60/613,267, filed on Sep. 27, 2004.

(51) Int. Cl. A61K 31/38; A61K 31/336; A61P 25/00

(52) U.S. Cl. 514/430; 514/475

(57) ABSTRACT

The invention provides methods to treat neurological disorders, opthalmological disorders, or a combination thereof by administering a compound that inhibits MMPs. A compound that inhibits MMPs is represented by the compound of formula (I) shown herein.

### RELATED APPLICATION

This application claims priority from U.S. Provisional Application Ser. No. 60/613,267 filed Sep. 27, 2004, which application is herein incorporated by reference.

### BACKGROUND OF THE INVENTION

Specific interactions of cells within the extracellular matrix are critical for the normal function of organisms. Alterations of the extracellular matrix are carried out by a family of zinc-dependent endopeptidases called matrix metalloproteinases (MMPs). The alterations are carried out in various cellular processes such as organ development, ovulation, fetus implantation in the uterus, embryo genesis, wound healing, and angiogenesis. Massova, I.; Kotra, L. P.; Fridman, R.; Mobashery, S. FASEB J. 1998,12,1075; Forget, M.-A.; Desrosier, R. R.; Béliveau, R. Can. J. Physiol. Pharmacol. 1999, 77, 465-480.

MMPs consist of five major groups of enzymes: gelatinases, collagenases, stromelysins, membrane-type MMPs and matrilysins. The activities of MMPs in normal tissue functions is strictly regulated by a series of complicated zymogen activation processes and inhibition by protein tissue inhibitors for matrix metalloproteinases (“TIMPs”). Forget, M.-A.; Desrosier, R. R.; Béliveau, R. Can. J. Physiol Pharmacol. 1999, 77, 465-480; Brew, K.; Dinakarpandian, D.; Nagase, H. Biochim. Biophys. Acta 2000, 1477, 267-283. Westermarck, J.; Kahari, V. M. FASEB J. 1999, 13, 781-792. Excessive MMP activity, when the regulation process fails, has been implicated in cancer growth, tumor metastasis, angiogenesis in tumors, arthritis and connective tissue diseases, cardiovascular disease, inflammation and autoimmune diseases. Massova, I.; Kotra, L. P.; Fridman, R.; Mobashery, S. FASEB J. 1998,12,1075; Forget, M.-A.; Desrosier, R. R.; Béliveau, R. Can. J. Physiol. Pharmacol. 1999, 77, 465-480; Nelson, A. R.; Fingleton, B.; Rothenberg, M. L.; Matrisian, L. M. J. Clin. Oncol. 2000, 18, 1135.

Increased levels of activity for the human gelatinases MMP-2 and MMP-9 have been implicated in the process of tumor metastasis. Dalberg, K.; Eriksson, E.; Enberg, U.; Kjellman, M.; Backdahl, M. World J. Surg. 2000,24, 334-340. Salo, T.; Liotta, L. A.; Tryggvason, K. J. Biol. Chem. 1983, 258, 3058-3063. Pyke, C; Ralfkiaer, E.; Huhtala, P.; Hurskainen, T.; Dano, K.; Tryggvason, K. Cancer Res. 1992, 52, 1336-1341. Dumas, V.; Kanitakis, J.; Charvat, S.; Euvrard, S.; Faure, M.; Claudy, A. Anticancer Res. 1999, 19, 2929-2938. As a result, select inhibitors of MMPs (e.g., MMP-2 and MMP-9) are highly sought.

Several competitive inhibitors of MMPs are currently known. These inhibitors of MMPs take advantage of chelation to the active site zinc for inhibition of activity. Because of this general property, these competitive inhibitors for MMPs are often toxic to the host, which has been a major impediment in their clinical use. Greenwald, R. A. Ann. N.Y. Acad. Sci. 1999, 878, 413-419; (a) Michaelides, M. R.; Curtin, M. L. Curr. Pharm. Des. 1999, 5, 787-819. (b) Beckett, R. P.; Davidson, A. H.; Drummond, A. H.; Huxley, P.; Whittaker, M. Drug Disc. Today 1996, 1, 16-26.

Accordingly, there is a current need for inhibitors of MMPs. Such inhibitors would be useful to treat diseases other than cancer. Preferred inhibitors may exhibit greater selectivity for one or more specific MMPs than known competitive inhibitors. Such methods will preferably not include negative long-term side-effects.

### SUMMARY OF THE INVENTION

The present invention provides a method for treating a neurological disorder, an opthalmological disorder, or a combination thereof in a mammal inflicted with a neurological disorder, an opthalmological disorder, or a combination thereof. The method includes administering to the mammal in need of such treatment an effective amount of a compound of formula (I) described herein.

The present invention also provides a method for treating a neurological disorder, an opthalmological disorder, or a combination thereof in a mammal inflicted with a neurological disorder, an opthalmological disorder, or a combination thereof. The method includes administering to the mammal in need of such treatment an effective amount of a matrix metalloproteinase (MMP) inhibitor.

The present invention also provides the use of a matrix metalloproteinase (MMP) inhibitor for treating a neurological disorder, an opthalmological disorder, or a combination thereof in a mammal inflicted with a neurological disorder, an opthalmological disorder, or a combination thereof.

The present invention further provides the use a compound of formula I or an MMP inhibitor for the manufacture of a medicament useful for treating a neurological disorder, opthalmological disorder, or a combination thereof in a mammal inflicted with a neurological disorder, an opthalmological disorder, or a combination thereof.

### BRIEF DESCRIPTION OF THE FIGURES

FIG. 1 illustrates a mechanism-based inhibition of an MMP by a compound useful in the present invention.

FIG. 2 illustrates bbsynthesis of compounds useful in the present invention.

FIG. 3 illustrates a mechanism-based inhibition of an MMP by a compound useful in the present invention.

FIG. 4 illustrates neuronal nitric oxide synthase (nNOS)-associated MMP-9 activation in ischemic cortex after middle cerebral artery (MCA) ischemia and reperfusion. (A) Top: Gelatin zymography showing increased proMMP-9 expression and MMP-9 activity on the ischemic side of the brain compared to the contralateral side after 2-hour MCA occlusion and 24-hour reperfusion in C57BL/6J mice (n=7). Middle: Immunoblotting with anti-MMP-9 antibody showing increased MMP-9 expression on the ischemic side of the mouse brain compared to the control side. Bottom: A slight decrease in actin on the ischemic side of the brain may reflect cell loss. The right MCA was occluded by intraluminal filament for 2 hours and then removed for reperfusion (Wang Y F, Tsirka S E, Strickland S, Stieg P E, Soriano S G, Lipton S A. Tissue plasminogen activator (tPA) increases neuronal damage after focal cerebral ischemia in wild-type and tPA-deficient mice. Nature Medicine 1998; 4:228-231). MMP-9 was extracted from brain tissue in Tris buffer (50 mM Tris, pH 7.6, 5 mM CaCl₂,150 mM NaCl, 0.05% Brij35) containing 1% Triton X-100, followed by affinity precipitation with Gelatin-Sepharose 4B (Gu Z, Kaul M, Yan B, Kridel S J, Cui J, Strongin A, Smith J W, Liddington R C, Lipton S A. S-Nitrosylation of matrix metalloproteinases: signaling pathway to neuronal cell death. Science 2002; 297:1186-1190). (B) In situ zymography with the MMP fluorogenic substrate DQ-gelatin-FITC (Molecular Probes, Eugene, Oreg.) was performed on fresh cryostat sections of mouse brains harvested post MCA ischemia and reperfusion. Deconvolution microscopy revealed increased MMP activity in the ischemic cortex compared to the control side (Untreated, C57BL/6J, top panel). Counterstaining with Hoechst 33342 showed decreased nuclear DNA staining, indicating cell loss in the ischemic cortex after ischemia and reperfusion. Increased MMP activity in the ischemic cortex was abrogated in mice injected intraperitoneally (i.p.) prior to ischemia with 3-bromo-7-nitroindazole (3br7NI, 30 mg/kg body weight, Alexis Biochemicals, San Diego, Calif.; control contained soybean oil vehicle; second panel) and in nNOS knockout mice (Jackson Laboratory, Bar Harbor, Me.; fourth panel), but not in wild-type control mice (third panel). (C) Neurons (NeuN immunopositive) double labeled for MMP activity (arrows) in the ischemic cortex. Nuclear DNA was visualized by staining with Hoechst 33342. Some nonneuronal cells also showed MMP activity (arrowheads). (D) Colocalizaton of nNOS and MMP-9 in the ischemic cortex was detected by double immunofluorescent staining after MCA ischemia and reperfusion. Scale bars, 50 μm.

FIG. 5 illustrates S-nitrosylation and consequent activation of MMP-9 in vitro by SNOC. (A) R-proMMP-9 (1.1 mg/ml) was incubated with SNOC (200 μM) for 15 min at room temperature. S-nitrosylated MMP-9 thus generated was assessed by release of NO causing the conversion of 2,3-diaminonaphthalene (DAN) to the fluorescent compound 2,3-naphthyltriazole (NAT) (*P< 0.03 by ANOVA). The concentration of S-nitrosothiol formation was detected by conversion of the fluorescent compound 2,3-naphthyltriazole (NAT) from 2,3-diaminonaphthalene (DAN) at an emission wavelength of 360 nm and an excitation wavelength of 260 nm using a FluoroMax-2 spectrofluorometer and DataMax software (Instruments S.A., Inc., Edison, N.J.) (Gu et al., ibid.). S-Nitrosocysteine (SNOC) itself quickly decayed and thus resulted in insignificant S-nitrosothiol readings in this assay (see also B). (B) Half-lives of S-nitroso-MMP-9 (λ, circles) and SNOC (σ, triangles). NO released from S-nitroso-MMP-9 or SNOC was detected by NAT conversion from DAN. The half-lives of SNOC and S-nitroso-MMP-9 were <10 min and ˜30 min, respectively. (C) Activation of proMMP-9 by APMA, SNOC, and acidified sodium nitrite (to yield nitrosonium, NO⁺). R-proMMP-9 (100 ng/ml) was reacted with 200 μM APMA, SNOC, acidified sodium nitrite, or L-cysteine for 18 hours at room temperature and subsequently analyzed by gelatin zymography. SNOC was generated by reaction of sodium nitrite and L-cysteine as described previously (see Gu et al., ibid., for references). The digested matrix, revealed by staining with Coomassie blue, indicated proteolytic activity. (D) Kinetics of activation of R-proMMP-9 treated with APMA (□, squares), SNOC (Δ, triangles), or untreated control (◯, circles). MMP activity was assessed by the cleavage rate of fluorogenic Substrate I Peptide (25 μM, Calbiochem, San Diego, Calif.; excitation wavelength, 280±1 nm; emission wavelength, 360±5 nm).

FIG. 6 illustrates that exogenous MMP-9 activated by SNOC induces neuronal apoptosis in cerebrocortical cell culture. (A) Neurons exhibiting MMP activity were identified by in situ zymography with the fluorogenic substrate DQ-gel-FITC, in combination with immunocytochemical staining using anti-microtubule associated protein-2 (MAP-2) antibody as a neuronal marker. Nuclear DNA was labeled with Hoechst 33342. (B) The percentage of MAP-2 positive neurons displaying MMP activity increased after exposure to ˜150 pM proMMP-9 that had been pre-activated with 200 μM SNOC (*P< 0.01 by Student's t-test, n= 1500 neurons counted in 5 separate experiments) (Gu et al., ibid.). (C) Apoptotic neurons were identified by staining with anti-MAP-2 and TUNEL in conjunction with nuclear morphology, as evaluated by DNA staining with Hoechst 33342. Scale bar, 20 μm. (D) Quantification of neuronal apoptosis induced by R-proMMP-9 pre-activated by SNOC prior to addition to cerebrocortical cultures for 18 hours. SNOC-activated MMP-9 significantly increased neuronal apoptosis, whereas the MMP inhibitor GM6001 abrogated the effect (*P< 0.01 by ANOVA, n= 4000 neurons scored in 6 experiments). NO was dissipated from old SNOC by overnight incubation prior to addition.

FIG. 7 illustrates peptide mass fingerprinting analysis by matrix-assisted laser desorption/ionization time-of-flight (MALDI-TOF) mass spectroscopy of the modified thiol group of the cysteine residue within the highly conserved auto-inhibitory prodomain of human and rodent MMP-9. (A) Top: R-proMMP-9 was subjected to SDS-PAGE under nonreducing conditions and visualized by Coomassie blue staining. The mass spectra from in-gel digested R-proMMP-9 revealed seven signature masses (arrows) from eleven tryptic fragments in the mass range of 700 to 1600 Da. The peak at 873.4 Da represents the peptide CGVPDLGR (SEQ ID NO:1) alkylated with iodoacetamide in the human prodomain fragment (acet-CGVPDLR; SEQ ID NO:2). Middle: After exposure of R-proMMP-9 to 200 M SNOC in vitro, MALDI-TOF mass spectrometry revealed a peak at 848.3 Da, representing SO₂H-CGVPDLGR (CGVPDLGR is represented by SEQ ID NO: 1). Bottom: Reduction of R-proMMP-9 with dithiothreitol (DTT) followed by alkylation with iodoacetamide prior to exposure to SNOC prevented the shift of the 873 Da peak representing acet-CGVPDLR (SEQ ID NO:2). (B) Top: MALDI-TOF spectra of in-solution tryptic digest of R-proMMP-9 revealed four signature masses from six tryptic fragments (arrows). Bottom: The tryptic fragment CGVPDLGR (SEQ ID NO:1) at 816.7 Da shifted by 48 Da to 864.8 Da (arrow) after exposure to SNOC, representing SO₃H-CGVPDLGR(CGVPDLGR is represented by SEQ ID NO:1). (C) Detection of tryptic fragments by MALDI-TOF mass spectrometry of gel-purified MMP-9 from rat brains following 2-hour middle cerebral artery (MCA) occlusion/15 min reperfusion. MMP-9 was extracted in Tris buffer with 1% Triton X-100, affinity precipitated with Gelatin-Sepharose 4B, subjected to SDS-PAGE gel under nonreducing conditions, and visualized by silver staining. Top: Gel-purified MMP-9 was reduced and alkylated prior to digestion. Detergent was removed as previously described (Gu et al., ibid., and references therein). MALDI-TOF mass spectrometry revealed a mass peak at 830.3 Da (arrow), representing the iodoacetamide (57 Da)-alkylated rat peptide acet-CGVPDVGK (57+774 Da; SEQ ID NO: 3) from the propeptide domain isolated from control brains. Bottom: A mass of 821.8 Da (arrow), representing the 774 Da propeptide domain fragment plus a 48 Da modification (SO₃H-CGVPDVGK; CGVPDVGK is represented by SEQ ID NO:3) was observed in the ischemic side of the brain. MALDI-TOF spectra did not detect modification of other cysteine residues within MMP-9 tryptic fragments.

FIG. 8 illustrates one model of MMP-9 activation by S-nitrosylation and subsequent oxidation. (A) Molecular surface of a partial sequence of human MMP-9 (from ⁹⁷Pro to ⁴¹¹His without the fibronectin repeats found between ²¹⁶Val and ³⁹¹Gln) (Morgunova, E.; Tuuttila, A.; Bergmann, U.; Isupov, M.; Lindqvist, Y.; Schneider, G.; Tryggvason, K. Science 1999, 284, 1667-1670; Toggas, S. M.; Masliah, E.; Rockenstein, E. M.; Rall, G. F.; Abraham, C. R.; Mucke, L. Nature 1994, 367, 188-193). Propeptide domain (⁹⁷Pro to ¹⁰⁶Arg) and catalytic domain (⁴⁰¹His to ⁴¹¹H is). In proMMP-9, Zn²⁺ is coordinated by a cysteine and three histidine residues. R98, C99, and E402 fit the proposed consensus motif for S-nitrosylation (Stamler, J. S.; Toone, E. J.; Lipton, S. A.; Sucher, N. J. Neuron 1997, 18, 691-696). R, Arg; C, Cys; E, Glu; H, His. (B, C) Proposed structure-based chemistry of NO-induced MMP-9 activation. Reactivity of the catalytic cysteine sulfur of MMP-9 appears to be enhanced by increased nucleophilicity of ⁴⁰²Glu to S-nitrosylating agents (SNOC=Cys-NO, for example). The sulfur bound at the zinc site appears to be highly nucleophilic, which may give high initial reactivity to NO from its endogenous donors. The S-nitroso-MMP-9 propeptide domain appears to be more easily broken up in this highly polar environment and replaced by a nucleophilic water molecule. Reaction with H₂O of the S-nitrosothiol group forms sulfenic acid (—SOH), as observed in glutathione reductase (Stamler, J. S.; Hausladen, A. Nature Structural Biology 1998, 5, 247-249; Becker, K.; Savvides, S, N.; Keese, M.; Schirmer, R. H.; Karplus, P. A. Nature Structural Biology 1998, 5, 267-271). The reversible sulfenic acid can serve as an intermediate leading to subsequent irreversible oxidation steps (at least in mammals) via ROS to sulfuric (—SO₂H) and sulfonic (—SO₃H) acids.

FIG. 9 illustrates the protective effects of the MMP-2/9 inhibitor SB3CT. SB3CT decreases infarct volume of mouse brains after a 2-hour focal middle cerebral artery occlusion (MCAO) and 24-hour reperfusion compared to normal saline with 10% DMSO-vehicle treated control. Coronal sections of 1-mm thickness were prepared and stained with TTC. (A) Representative TTC staining of sections of mouse brains. SB3CT was administrated intraperitoneally as a suspension (25 mg/kg body weight per treatment). Mice were treated twice, 30 minutes before MCAO and 2 hours after MCAO. (B) Infarct volumes were determined at 24 hours after reperfusion and quantified with NIH Image (version 6.2) using methods to exclude possible confounding effects of brain swelling. Data represent mean±SEM. (Numbers of animals in each group: Vehicle, n=8; SB3CT, n=5; *P< 0.004). (C) Neurological behavioral outcomes 24 hours after focal MCAO and reperfusion. Mice were tested and scored for neurological deficits as follows: 0= no observable neurological deficit; 1= failure to extend left forepaw; 2= spontaneous circling to the left; 3= falling to the left; 4=cannot walk spontaneously. Treatment with SB3CT (n=13) significantly reduced neurological deficit outcomes comparing to the vehicle-treated control group (n= 11); P< 0.01 by Student's t-test.

FIG. 10 illustrates Laser-Doppler flowmetry of regional cerebral blood flow (rCBF). For each mouse, rCBF decreased to less than 25% of the baseline value during a 2-hour ischemia period of MCAO, and recovered to more than 50% of baseline during reperfusion. There were no significant differences in rCBF between vehicle-treated controls (solid squares, n= 11) and SB3CT treatment (solid triangles, n= 13). Ordinate values are divided by 100 (e.g., 1=100%).

FIG. 11 illustrates that SB3CT attenuates activation of MMP-9. In situ zymography with the MMP fluorogenic substrate DQ-gelatin-FITC (Molecular Probes) was performed on fresh cryostat sections of mouse brains harvested after MCA ischemia and reperfusion (A to J). (A and B) Increased MMP activity in the ischemic cortex. (C to H) MMP activity was reduced by MMP inhibitors (GM6001 in panels C and D, and 1,10-phenanthroline in E and F), but not by a cocktail of non-MMP inhibitors (protease inhibitor cocktail, Sigma P-8340, in G and H). (I and J) Deconvolution images of ischemic brains treated with SB3CT (J) compared to vehicle-treated control (I). A, C, E, and G represent fluorogenic substrate (green), reflecting MMP activity in situ. B, D, F, H, I, and J are merged images of the fluorogenic substrate and Hoechst dye counterstaining to identify nuclei. Other sections (not shown here, but published in Science paper (Gu et al., Ibid) demonstrate that much of the MMP activity is associated with neurons (NeuN positive cells). (K) Gelatin zymography showing increased pro-MMP-9 expression (dense band) and MMP-9 activity (faint band) on the ischemic side of the brain compared with the contralateral side after 2-hr MCA occlusion and 24-hr reperfusion. SB3CT attenuated the increase in pro-MMP-9 expression and active MMP-9.

FIG. 12 illustrates colocalization of MMP activity with neuronal laminin and association with neuronal apoptosis in the ischemic cortex. Column 1 shows colocalization of neurons (A1, NeuN immunoreactivity) with laminin (B1, poly-Laminin pAb from Sigma, catalog #L-9393), nuclear labeling with Hoechst dye 33342 (C1), and merged image (D1). Note the elongated laminin label represents blood vessels that are labeled in addition to neurons. Column 2 shows in situ MMP activity by zymography (A1) colabeling with laminin (B2), nuclear labeling with Hoechst, and the merged image (D2). Column 3 shows in situ MMP activity by zymography (A3), colabeling with TTJNEL (B3), apoptotic morphology by Hoechst (C3), and merged image (D3). Scale bar, 20 μm.

FIG. 13 illustrates degradation of laminin correlates with neuronal apoptosis. (A and B) Coronal brain sections were stained for laminin immunoreactivity and TUNEL to demonstrate the reduction of laminin in the ischemic cortex surrounding apoptotic-appearing cells. Panel (B) represents ischemic cortex and (A), the contralateral control hemisphere. Brain sections were counterstained with Hoechst dye to show nuclear morphology. (C) The specific MMP-2/9 inhibitor, SB3CT, attenuated laminin degradation products (arrowhead at bottom of western blot) in the ischemic hemisphere. The lower blot is the same as the upper but developed longer to demonstrate the laminin degradation bands more clearly.

FIG. 14 illustrates that thiirane inhibitor SB-3CT protects against brain damage and ameliorates neurological outcome after transient focal cerebral ischemia in mice. A, Laser Doppler flowmetry of rCBF. rCBF was measured over the ischemic core of the right MCA region, and the preischemic rCBF was assigned a value of 100% at baseline. For each mouse, rCBF decreased to 25% of the baseline value during a 2 h period of ischemia and recovered to 50% of baseline during reperfusion. There were no significant differences in rCBF between vehicle-treated controls (solid squares; n 11) and SB-3CT-treated animals (solid triangles represent mean SEM; n=13). B, Representative TTC staining of stroke in mouse-brain sections after SB-3CT treatment versus vehicle-treated control (Vehicle). SB-3CT (25 mg/kg body weight per treatment) was administrated intraperitoneally as a suspension in a vehicle solution (10% DMSO in saline). SB-3CT was administered in four groups plus parallel vehicle-treated control groups: a preischemic group treated 0.5 h before insult (0.5 h) and groups treated 2, 6, or 10 h after ischemia (labeled 2, 6, and 10 h; see Materials and Methods). Coronal sections, 1 mm in thickness, were prepared and stained with TTC. C, Quantification of infarct volume by TTC staining. Infarct volumes were determined 24 h after reperfusion. SB-3CT decreased infarct volume compared with vehicle-treated controls (Ctrl). Data represent mean SEM. The numbers of animals in each group are as follows: Ctrl, n=19; SB-3CT, n=23. *p<0.001 by ANOVA. D, Neurological behavioral score (see Materials and Methods) 24 h after MCA occlusion/reperfusion. Treatment with SB-3CT (n=31) significantly improved neurological function compared with vehicle-treated controls (n 22); *p<0.02 by ANOVA. Error bars represent SEM.

FIG. 15 illustrates that thiirane inhibitor SB-3CT inhibits MMP-9 activity and consequent increased expression of MMP-9 in the ischemic mouse brain after transient middle cerebral artery occlusion. A, In situ zymography with the MMP fluorogenic substrate DQ-gel (green in top panels) merged with nuclear DNA staining by Hoechst dye (blue plus green in bottom panels). The broad-spectrum MMP inhibitors 1,10-phenanthroline and GM6001, but not a non-MMP PIC, abrogated MMP gelatinolytic activity in the ischemic cortex after MCA occlusion/reperfusion. Scale bar, 25 μm. B, SB-3CT significantly reduced MMP gelatinolytic activity in the ischemic region compared with the vehicle-treated control, as demonstrated by deconvolution microscopy. C, Gelatin zymography and Western blotting reveal upregulation of proMMP-9 (92 kDa) and activation of MMP-9 (act.MMP-9) in the ischemic brain compared with the contralateral hemisphere. In contrast, MMP-2 was not affected. SB-3CT attenuated the increase in proMMP-9 and act.MMP-9. Actin was used as a loading control. D, Quantification of relative MMP-9 activity by densitometry of gelatin zymography. Vehicle, n=8; SB-3CT, n=6; *p< 0.0001. Error bars represent SEM.

FIG. 16 illustrates increased MMP gelatinolytic activity is spatially associated with neuronal laminin in the ischemic cortex of mouse brains after transient middle cerebral artery occlusion. Left panels, Double-immunofluorescent staining revealed two types of morphology, representing Ln (red) on elongated microvascular structures and on the neuronal surface (neurons labeled with the neuron-specific marker anti-NeuN; green). Top left, Inset, Immunolabeling of the α-5 subunit of laminin-10 (Lnα5), which is specific to neurons (Indyk et al., 2003). Right panels, Increased MMP gelatinolytic activity (DQ-gel; green) colocalized with laminin detected by immunostaining with a pan-Ln antibody (red) in the ischemic cortex 2 h after reperfusion. Bottom panels, Merged images counterstained with Hoechst dye to visualize nuclei (blue). Scale bar, 25 μm.

FIG. 17 illustrates that exogenous MMP-9 degrades laminin in the extracellular matrix protein of mouse brain. A, Western blot with a pan-Ln polyclonal antibody reveals degradation of laminin (especially the 360 and 170 kDa subunits) to a 51 kDa fragment (frag.) in brain lysates treated with activated MMP-9 but not with latent proMMP-9 or catalytic MT1-MMP (50 g of total protein per lane). Purified mouse Engelbreth-Holm-Swarm laminin (ms EHS Ln) served as a molecular marker for laminin immunoblotting. The membrane was reblotted with anti-actin antibody to ensure equal protein loading in each lane. B, Ex vivo degradation of neuronal laminin by exogenous MMP-9 in mouse-brain sections. Double immunolabeling of laminin by pan-Ln polyclonal antibody (Ln; red) and neurons with NeuN antibody (green) reveals that activated MMP-9 degraded neuronal laminin. A broad-spectrum MMP inhibitor, GM6001, significantly reduced laminin degradation, whereas a non-MMP PIC did not. Latent proMMP-9 or catalytic MT1-MMP could not degrade neuronal laminin. Merged images were counterstained with Hoechst dye to visualize nuclei (blue). Scale bar, 25 μm.

FIG. 18 illustrates that NO-activated MMP-9 leads to laminin degradation in the ischemic cortex after MCA occlusion/reperfusion. A, Laminin immunoreactivity (red) and Hoechst DNA stain (blue). Deconvolution microscopy revealed that laminin immunoreactivity was significantly reduced in the ischemic cortex of wild-type mice (top right) compared with the contralateral nonischemic control hemisphere (top left). Laminin degradation in the ischemic cortex was attenuated after MCA occlusion/reperfusion in either wild-type mice treated with the specific nNOS inhibitor 3-bromo-7-nitroindazole (3br7NI; bottom left) or in nNOS KO mice (bottom right). Scale bar, 25 μm. B, Quantification of Ln-positive cells in cortex was determined 24 h after reperfusion. Data represent mean SEM on 600-1000 cells counted from each brain section (n=5 in each group; *p<0.001 compared with control group and #p<0.001 compared with ischemic group by ANOVA). Error bars represent SEM.

FIG. 19 illustrates the time course of laminin degradation and apoptotic cell death in the ischemic cortex after transient MCAO/R in mice. A-C, In situ zymography reveals that increased MMP gelatinolytic activity (A; green) is associated with apoptotic cell death detected by TUNEL (B; red). Merged images were counterstained with Hoechst dye to visualize nuclei (C; blue). D-F, After 2 h focal cerebral ischemia plus 3 h reperfusion (E) or 24 h reperfusion (F), animals were killed, and the brains were processed for immunohistochemistry. Coronal brain sections were stained for laminin immunoreactivity (red) and nuclear DNA staining with Hoechst dye (blue). D, Immunostaining with anti-pan-Ln antibody (red; indicated by arrowheads) was decreased in the ischemic cortex as early as 3 h after reperfusion compared with the control contralateral cortex. The remaining laminin was mostly associated with elongated microvascular structures rather than neurons. Modest neuronal cell death occurred at 3 h but more massive death at 24 h (arrows), as evidenced by condensed nuclei. G, Quantification of Ln-positive and apoptotic cells in the cortex 24 h after reperfusion. Data represent mean±SEM on 600-1000 cells counted from each brain section; n=5 in each group; *p<0.001 by ANOVA compared with Ln-positive cells in the control contralateral hemisphere (Ctrl; filled bar), and #p<0.001 compared with apoptotic cells in the control contralateral hemisphere (Ctrl; open bar). H, I, Coronal brain sections were stained for laminin immunoreactivity (green) and TUNEL (red) to demonstrate the reduction in laminin and increase in apoptosis in the ischemic cortex (I) compared with the contralateral control cortex (H), Brain sections were counterstained with Hoechst dye to show nuclei (blue). Together, the data in this figure suggest that MMP-induced laminin degradation occurs before neuronal apoptotic-like cell death. Scale bar, 25 μm. Error bars represent SEM.

FIG. 20 illustrates that SB-3CT attenuates laminin degradation in the ischemic hemisphere after MCAO/R. Western blot demonstrates laminin proteolysis (especially of the 360 and 170 kDa subunits) to a 51 kDa fragment in the ischemic brain (arrowhead at bottom of gel), whereas treatment with SB-3CT decreased laminin degradation after transient MCAO/R. The 60 kDa fragment may represent an additional proteolytic derivative of the subunit (bottom molecular band) lacking NH₂-terminal residues, as reported previously (Giannelli et al., 1997). The membrane was reprobed with anti-actin antibody to ensure equal loading.

FIG. 21 illustrates that disruption of laminin-cell surface interactions increases sensitivity to ischemic death. Mouse brains were infused with normal rabbit serum (IgG) or with a neutralizing antibody to pan-laminin (anti-Ln) in 1% BSA/PBS for 2 d before MCAO/R plus SB-3CT treatment or vehicle only. Brain sections were stained with cresyl violet and acid fuchsin. The dashed red line encircles the area of cell death. Pan-laminin antibody increased cell death in the MCAO/R mouse model despite SB-3CT treatment. This finding is consistent with the notion that the action of anti-laminin antibody is downstream to MMP-9 activation. Scale bar, 1 mm.

### DETAILED DESCRIPTION OF THE INVENTION

The following definitions are used, unless otherwise described: halo is fluoro, chloro, bromo, or iodo. Alkyl, alkoxy, alkenyl, alkynyl, etc. denote both straight and branched groups; but reference to an individual group such as “propyl” embraces only the straight chain variant, a branched chain isomer such as “isopropyl” being specifically referred to. Bicyclic aryl denotes an ortho-fused bicyclic carbocyclic substituent having about nine to ten ring atoms in which at least one ring is aromatic. Monocyclic heteroaryl encompasses a substituent attached via a ring carbon of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl. Bicyclic heteroaryl encompasses a substituent of an ortho-fused bicyclic heterocycle of about eight to ten ring atoms derived therefrom, particularly a benzyl-derivative or one derived by fusing a propylene, trimethylene, or tetramethylene divalent substituent thereto. Bicyclic alkyl encompasses a substituent of an ortho-fused bicyclic alkyl of about eight to ten ring atoms containing five or six ring atoms consisting of carbon and one to four ring atoms consisting of heteroatoms selected from the group consisting of non-peroxide oxygen, sulfur, and N(X) wherein X is absent or is H, O, (C₁-C₄)alkyl, phenyl or benzyl.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine MMP inhibition activity using the standard tests described herein below, or using other similar tests which are well known in the art.

Specific and preferred values listed below for substituents (i.e., groups) and ranges are for illustration only; they do not exclude other defined values or other values within defined ranges for the substituents

Specifically, (C₁-C₆)alkyl can be methyl, ethyl, propyl, isopropyl, butyl, iso-butyl, sec-butyl, pentyl, 3-pentyl, or hexyl;

(C₁-C₆)alkoxy can be methoxy, ethoxy, propoxy, isopropoxy, butoxy, iso-butoxy, sec-butoxy, pentoxy, 3-pentoxy, or hexyloxy;

(C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl;

(C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl;

(C₁-C₆)alkanoyl can be acetyl, propanoyl or butanoyl;

(C₂-C₆)alkanoyloxy can be acetoxy, propanoyloxy, butanoyloxy, isobutanoyloxy, pentanoyloxy, or hexanoyloxy;

(C₃-C₈)cycloalkyl can be cyclopropyl, cyclobutyl, cyclopentyl, cyclohexyl, cycloheptyl, or cyclooctyl; aryl can be phenyl, indenyl, 5,6,7,8-tetrahydronaphthyl, or naphthyl and heteroaryl can be furyl, imidazolyl, tetrazolyl, pyridyl, (or its N-oxide), thienyl, pyrimidinyl (or its N-oxide), indolyl, or quinolyl (or its N-oxide); bicyclic aryl can be indenyl or naphthyl; monocyclic heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl (or its N-oxide), thienyl, or pyrimidinyl (or its N-oxide), bicyclic heteroaryl can be quinolyl (or its N-oxide); and bicyclic alkyl can be decahydroquinoline or decahydronaphthalene (cis and trans).

As used herein, “treating” or “treat” includes (i) preventing a pathologic condition (e.g., neurological and/or an opthalmological disorder) from occurring; (ii) inhibiting the pathologic condition (e.g., neurological and/or an opthalmological disorder) or arresting its development; (iii) relieving the pathologic condition (e.g., neurological and/or an opthalmological disorder), or (iv) alleviating the symptoms associated with the pathologic condition (e.g., neurological and/or an opthalmological disorder).

As used herein, an “amino acid” is a natural amino acid residue (e.g., Ala, Arg, Asn, Asp, Cys, Glu, Gln, Gly, His, Hyl, Hyp, He, Leu, Lys, Met, Phe, Pro, Ser, Thr, Trp, Tyr, and Val) in D or L form, as well as unnatural amino acid (e.g., phosphoserine; phosphothreonine; phosphotyrosine; hydroxyproline; gamma-carboxyglutamate; hippuric acid; octahydroindole-2-carboxylic acid; statine; 1,2,3,4,-tetrahydroisoquinoline-3-carboxylic acid; penicillamine; ornithine; citruline; α-methyl-alanine; para-benzoylphenylalanine; phenylglycine; propargylglycine; sarcosine; and tert-butylglycine) residue having one or more open valences. The term also comprises natural and unnatural amino acids bearing amino protecting groups (e.g. acetyl, acyl, trifluoroacetyl, or benzyloxycarbonyl), as well as natural and unnatural amino acids protected at carboxy with protecting groups (e.g., as a (C₁-C₆)alkyl, phenyl or benzyl ester or amide). Other suitable amino and carboxy protecting groups are known to those skilled in the art (See for example, T. W. Greene, Protecting Groups In Organic Synthesis: Wiley: New York, 1981; D. Voet, Biochemistry. Wiley: New York, 1990; L. Stryer, Biochemistry. (3rd Ed.), W.H. Freeman and Co.: New York, 1975; J. March, Advanced Organic Chemistry, Reactions, Mechanisms and Structure. (2nd Ed.), McGraw Hill: New York, 1977; F. Carey and R. Sundberg, Advanced Organic Chemistry, Part B: Reactions and Synthesis, (2nd Ed.), Plenum: New York, 1977; and references cited therein). According to the invention, the amino or carboxy protecting group can also comprise a radionuclide (e.g., Fluorine-18, Iodine-123, or Iodine-124).

As used herein, an “electrophile” refers to a chemical species, ion, or a portion of a compound which, in the course of a chemical reaction, will acquire electrons, or share electrons, to form other molecules or ions. Electrophiles are ordinarily thought of as cationic species (positively charged). McGraw-Hill Concise Encyclopedia of Science & Technology. McGraw-Hill, p. 715, 4^{th} Edition, NY, N.Y. (1998).

As used herein, a “nucleophile” refers to a chemical species, ion, or a portion of a compound which, in the course of a chemical reaction, will lose electrons, or share electrons, to form other molecules or ions. Nucleophiles are ordinarily thought of as anionic species (negatively charged). Typical nucleophilic species include, e.g., hydroxyl (OH⁻), halo (F⁻, Cl⁻, Br⁻, or I⁻), cyano (CN⁻), alkoxy (CH₃CH₂O⁻), carboxyl (COO⁻), and thio (S⁻). McGraw-Hill Concise Encyclopedia of Science & Technology, McGraw-Hill, p. 715, 4^{th} Edition, NY, N.Y. (1998).

As used herein, a “peptide” is a sequence of 2 to 25 amino acids (e.g., as defined hereinabove) or peptidic residues having one or more open valences. The sequence may be linear or cyclic. For example, a cyclic peptide can be prepared or may result from the formation of disulfide bridges between two cysteine residues in a sequence. A peptide can be linked through the carboxy terminus, the amino terminus, or through any other convenient point of attachment, such as, for example, through the sulfur of a cysteine. Peptide derivatives can be prepared as disclosed in U.S. Pat. Nos. 4,612,302; 4,853,371; and 4,684,620. Peptide sequences specifically recited herein are written with the amino terminus on the left and the carboxy terminus on the right.

As used herein, a “hydrophobic group” or “hydrophobic moiety” refers to a group that is relatively non-polar and will have a relatively minimal affinity for water. The nature of the hydrophobic group (i.e., A-X-M) is not important, provided the hydrophobic group fits into the pocket and has a favorable interaction (e.g., binding) with the enzyme. The hydrophobic group, while being relatively hydrophobic, can include one or more heteroatoms (e.g., S, O, or N) that can have an electrostatic charge or can include one or more groups (e.g., esters or amides) that can have an electrostatic charge, provided the hydrophobic group fits into the pocket and has a favorable interaction with the enzyme.

Any suitable hydrophobic group can be employed as A-X-M, provided the hydrophobic group fits into the pocket and has a favorable interaction (e.g., binding) with the enzyme. For example, the hydrophobic group can include a straight-chained or branched hydrocarbon chain (e.g., alkyl, alkenyl, or alkynyl), an aryl group (e.g., monocyclic or bicyclic), a heteroaryl group (e.g., monocyclic or bicyclic), a cycloalkyl group, an amino acid, a peptide, or a combination thereof.

In one embodiment, A-X-M can be a saturated or partially unsaturated hydrocarbon chain comprising one or more carbon atoms and optionally comprising one or more oxy (—O—), thio (—S—), sulfinyl (—SO—), sulfonyl (S(O)₂—), or NR_{f} in the chain, wherein each R_{f} is independently hydrogen or (C₁-C₆)alkyl. The saturated or partially unsaturated hydrocarbon chain can optionally be substituted with one or more oxo (═O), hydroxy, cyano, halo, nitro, trifluoromethyl, trifluoromethoxy, (C₁-C₆)alkyl, (C₁-C₆)alkoxy, (C₁-C₆)alkoxy(C₁-C₆)alkyl, (C₃-C₈)cycloalkyl, aryl, heteroaryl, (C₃-C₈)cycloalkyl(C₁-C₆)alkyl, (aryl)(C₁-C₈)alkyl, (heteroaryl)(C₁-C₆)alkyl, (C₃-C₈)cycloalkyl oxy, (aryl)oxy, (heteroaryl)oxy, (C₃-C₈)cycloalkyl, (aryl)oxy(aryl), (heteroaryl)oxy(heteroaryl), (C₃-C₈)cycloalkyl oxy (C₁-C₆)alkyl, (aryl)oxy (C₁-C₆)alkyl, or (heteroaryl)oxy (C₁-C₆)alkyl. In addition, any aryl, (C₃-C₈)cycloalkyl, or heteroaryl can optionally be substituted with one or more oxo (═O), hydroxy, cyano, halo, nitro, trifluoromethyl, trifluoromethoxy, (C₁-C₆)alkyl, (C₁-C₆)alkoxy, (C₁-C₆)alkoxy(C₁-C₆)alkyl, (C₃-C₈)cycloalkyl, aryl, heteroaryl, (C₃-C₈)cycloalkyl (C₁-C₆)alkyl, (aryl)(C₁-C₈)alkyl, (heteroaryl)(C₁-C₆)alkyl, (C₃-C₈)cycloalkyl oxy, (aryl)oxy, (heteroaryl)oxy, (C₃-C₈)cycloalkyl, (aryl)oxy(aryl), (heteroaryl)oxy(heteroaryl), (C₃-C₈)cycloalkyl oxy (C₁-C₆)alkyl, (aryl)oxy (C₁-C₆)alkyl, or (heteroaryl)oxy (C₁-C₆)alkyl.

When A-X-M is a “partially unsaturated” group, such group may comprise one or more (e.g., 1 or 2) carbon-carbon double or triple bonds. For example, when A-X-M is a partially unsaturated (C₁-C₆)alkyl, it can be vinyl, allyl, 1-propenyl, 2-propenyl, 1-butenyl, 2-butenyl, 3-butenyl, 1,3-butadienyl, 1-pentenyl, 2-pentenyl, 3-pentenyl, 4-pentenyl, 1-hexenyl, 2-hexenyl, 3-hexenyl, 4-hexenyl, 2,4-hexadienyl, 5-hexenyl, ethynyl, 1-propynyl, 2-propynyl, 1-butynyl, 2-butynyl, 3-butynyl, 1-pentynyl, 2-pentynyl, 3-pentynyl, 4-pentynyl, 5-hexene-1-ynyl, 2-hexynyl, 3-hexynyl, 3-hexen-5-ynyl, 4-hexynyl, or 5-hexynyl.

A specific value for A-X-M is A and M are each independently phenyl or monocyclic heteroaryl, wherein any phenyl or heteroaryl is optionally substituted with one or more (e.g., 1,2, 3, or 4) hydroxy, (C₁-C₆)alkyl, (C₁-C₆)alkanoyl, (C₁-C₆)alkanoyloxy, (C₁-C₆)alkoxy, cyano, nitro, halo, trifluoromethyl, trifluoromethoxy, SR, NRR, or COOR; and

X is O, S, SO, SO₂, C(═O)NR, C(═O)O, NRC(═O), OC(═O), NR, a direct bond, or (C₁-C₆)alkyl optionally substituted with one or more hydroxy, (C₁-C₆)alkoxy, cyano, nitro, halo, SR, NRR, or COOR.

Another specific value for A-X-M is bicyclic aryl (e.g., naphthyl), bicyclic heteroaryl, or bicyclic alkyl; wherein any aryl, heteroaryl or alkyl is optionally substituted with one or more (e.g., 1,2, 3, or 4) hydroxy, (C₁-C₆)alkyl, (C₁-C₆)alkanoyl, (C₁-C₆)alkanoyloxy, (C₁-C₆)alkoxy, cyano, nitro, halo, trifluoromethyl, trifluoromethoxy, SR, NRR, or COOR;

wherein each R is independently H, (C₁-C₆)alkyl, phenyl, benzyl, or phenethyl.

A specific value for A is phenyl or monocyclic heteroaryl. Another specific value for A is phenyl.

A specific value for M is phenyl or monocyclic heteroaryl. Another specific value for M is phenyl.

A specific value for X is O, S, SO, SO₂, C(═O)NR, C(═O)O, NRC(═O), OC(═O), NR, a direct bond, or (C₁-C₆)alkyl. Another specific value for X is O.

Another specific value for A-X-M is:

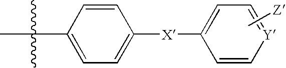

wherein

X′ is O, (C₁-C₆)alkyl (e.g., CH₂), or a direct bond;

Y′ is N or (C₁-C₆)alkyl (e.g., CH₂); and

Z′ is halo, (C₁-C₆)alkoxy (e.g., OCH₃), or hydroxy.

Another specific value for A-X-M is:

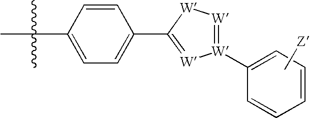

wherein

each W′ is independently N or CH; and

Z′ is halo, (C₁-C₆)alkoxy (e.g., OCH₃), or hydroxy.

Another specific value for A-X-M is:

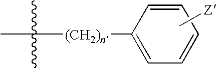

wherein

n′ is about 1 to about 4; and

Z′ is halo, (C₁-C₆)alkoxy (e.g., OCH₃), or hydroxy,

Another specific value for A-X-M is:

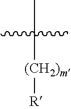

wherein

R′ is 0, (C₁-C₆)alkyl (e.g., CH₂), or S; and

m′ is about 2 to about 7.

Another specific value for A-X-M is:

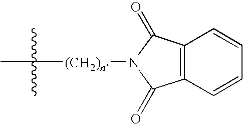

wherein

n′ is about 1 to about 4.

Another specific value for A-X-M is:

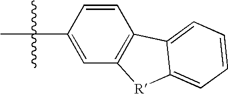

wherein

R′ is O, CH₂, or S.

A specific value for D is SO₂.

A specific value for E is (C₁-C₆)alkyl. Another specific value for E is methyl.

A specific value for (C₁-C₆)alkyl is methyl.

A specific value for J is S.

A specific value for G is hydrogen.

A specific value for T is hydrogen.

A specific value for Q is hydrogen.

A specific compound of the present invention is a compound of formula (I):

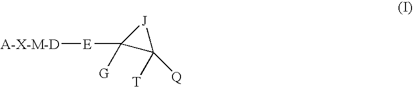

wherein A is phenyl, M is phenyl, X is O, D is SO₂, E is methyl, J is S, G is hydrogen, T is hydrogen, and Q is hydrogen.

As used herein, “neurological disorder” refers to any disorder of the nervous system and/or visual system. “Neurological disorders” include disorders that involve the central nervous system (brain, brainstem and cerebellum), the peripheral nervous system (including cranial nerves), and the autonomic nervous system (parts of which are located in both central and peripheral nervous system). Major groups of neurological disorders include, but are not limited to, headache, stupor and coma, dementia, seizure, sleep disorders, trauma, infections, neoplasms, neuroopthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological disorder. The following is a list of several neurological disorders, symptoms, signs and syndromes: Acquired Epileptiform Aphasia; Acute Disseminated Encephalomyelitis; Adrenoleukodystrophy; Agenesis of the corpus callosum; Agnosia; Aicardi syndrome; Alexander disease; Alpers' disease; Alternating hemiplegia; Alzheimer's disease; Amyotrophic lateral sclerosis; Anencephaly; Angelman syndrome; Angiomatosis; Anoxia; Aphasia; Apraxia; Arachnoid Cysts; Arachnoiditis; Arnold-Chiari malformation; Arteriovenous malformation; Asperger syndrome; Ataxia Telangiectasia; Attention Deficit Hyperactivity Disorder; Autism; Autonomic Dysfunction; Back Pain; Batten disease; Behcet's disease; Bell's palsy; Benign Essential Blepharospasm; Benign Focal; Amyotrophy; Benign Intracranial Hypertension; Binswanger's disease; Blepharospasm; Bloch Sulzberger syndrome; Brachial plexus injury; Brain abscess; Brain injury; Brain tumors (including Glioblastoma multiforme); Spinal tumor; Brown-Sequard syndrome; Canavan disease; Carpal tunnel syndrome (CTS); Causalgia; Central pain syndrome; Central pontine myelinolysis; Cephalic disorder; Cerebral aneurysm; Cerebral arteriosclerosis; Cerebral atrophy; Cerebral gigantism; Cerebral palsy; Charcot-Marie-Tooth disease; Chemotherapy-induced neuropathy and neuropathic pain; Chiari malformation; Chorea; Chronic inflammatory demyelinating polyneuropathy (CTDP); Chronic pain; Chronic regional pain syndrome; Coffin Lowry syndrome; Coma, including Persistent Vegetative State; Congenital facial diplegia; Corticobasal degeneration; Cranial arteritis; Craniosynostosis; Creutzfeldt-Jakob disease; Cumulative trauma disorders; Cushing's syndrome; Cytomegalic inclusion body disease (CIBD); Cytomegalovirus Infection; Dancing eyes-dancing feet syndrome; Dandy-Walker syndrome; Dawson disease; De Morsier's syndrome; Dejerine-Klumpke palsy; Dementia; Dermatomyositis; Diabetic neuropathy; Diffuse sclerosis; Dysautonomia; Dysgraphia; Dyslexia; Dystonias; Early infantile epileptic encephalopathy; Empty sella syndrome; Encephalitis; Encephaloceles; Encephalotrigeminal angiomatosis; Epilepsy; Erb's palsy; Essential tremor; Fabry's disease; Fahr's syndrome; Fainting; Familial spastic paralysis; Febrile seizures; Fisher syndrome; Friedreich's ataxia; Fronto-Temporal Dementia and other “Tauopathies”; Gaucher's disease; Gerstmann's syndrome; Giant cell arteritis; Giant cell inclusion disease; Globoid cell Leukodystrophy; Guillain-Barre syndrome; HTLV-1 associated myelopathy; Hallervorden-Spatz disease; Head injury; Headache; Hemifacial Spasm; Hereditary Spastic Paraplegia; Heredopathia atactica polyneuritiformis; Herpes zoster oticus; Herpes zoster; Hirayama syndrome; HIV-Associated Dementia and Neuropathy (see also Neurological manifestations of AIDS); Holoprosencephaly; Huntington's disease and other polyglutamine repeat diseases; Hydranencephaly; Hydrocephalus; Hypercortisolism; Hypoxia; Immune-Mediated encephalomyelitis; Inclusion body myositis; Incontinentia pigmenti; Infantile; phytanic acid storage disease; Infantile Refsum disease; Infantile spasms; Inflammatory myopathy; Intracranial cyst; Intracranial hypertension; Joubert syndrome; Kearns-Sayre syndrome; Kennedy disease; Kinsbourne syndrome; Klippel Feil syndrome; Krabbe disease; Kugelberg-Welander disease; Kuru; Lafora disease; Lambert-Eaton myasthenic syndrome; Landau-Kleffner syndrome; Lateral medullary (Wallenberg) syndrome; learning disabilities; Leigh's disease; Lennox-Gastaut syndrome; Lesch-Nyhan syndrome; Leukodystrophy; Lewy body dementia; Lissencephaly; Locked-In syndrome; Lou Gehrig's disease (aka Motor Neuron Disease or Amyotrophic Lateral Sclerosis); Lumbar disc disease; Lyme disease-Neurological Sequelae; Machado-Joseph disease; Macrencephaly; Megalencephaly; Melkersson-Rosenthal syndrome; Menieres disease; Meningitis; Menkes disease; Metachromatic leukodystrophy; Microcephaly; Migraine; Miller Fisher syndrome; Mini-Strokes; Mitochondrial Myopathies; Mobius syndrome; Monomelic amyotrophy; Motor Neurone Disease; Moyamoya disease; Mucopolysaccharidoses; Multi-Infarct Dementia; Multifocal motor neuropathy; Multiple sclerosis and other demyelinating disorders; Multiple system atrophy with postural hypotension; Muscular dystrophy; Myasthenia gravis; Myelinoclastic diffuse sclerosis; Myoclonic encephalopathy of infants; Myoclonus; Myopathy; Myotonia congenital; Narcolepsy; Neurofibromatosis; Neuroleptic malignant syndrome; Neurological manifestations of AIDS; Neurological sequelae of lupus; Neuromyotonia; Neuronal ceroid lipofuscinosis; Neuronal migration disorders; Niemann-Pick disease; O'Sullivan-McLeod syndrome; Occipital Neuralgia; Occult Spinal Dysraphism Sequence; Ohtahara syndrome; Olivopontocerebellar Atrophy; Opsoclonus Myoclonus; Optic neuritis; Orthostatic Hypotension; Overuse syndrome; Paresthesia; Parkinson's disease; Paramyotonia Congenita; Paraneoplastic diseases; Paroxysmal attacks; Parry Romberg syndrome; Pelizaeus-Merzbacher disease; Periodic Paralyses; Peripheral Neuropathy; Painful Neuropathy and Neuropathic Pain; Persistent Vegetative State; Pervasive developmental disorders; Photic sneeze reflex; Phytanic Acid Storage disease; Pick's disease; Pinched Nerve; Pituitary Tumors; Polymyositis; Porencephaly; Post-Polio syndrome; Postherpetic Neuralgia (PHN); Postinfectious Encephalomyelitis; Postural Hypotension; Prader-Willi syndrome; Primary Lateral Sclerosis; Prion diseases; Progressive; Hemifacial Atrophy; Progressive multifocal leukoencephalopathy; Progressive Sclerosing Poliodystrophy; Progressive Supranuclear Palsy; Pseudotumor cerebri; Ramsay-Hunt syndrome (Type I and Type II); Rasmussen's Encephalitis; Reflex Sympathetic Dystrophy syndrome; Refsum disease; Repetitive Motion Disorders; Repetitive Stress Injuries; Restless Legs syndrome; Retro virus-Associated Myelopathy; Rett syndrome; Reye's syndrome; Saint Vitus Dance; Sandhoff disease; Schilder's disease; Schizencephaly; Septo-Optic Dysplasia; Shaken Baby syndrome; Shingles; Shy-Drager syndrome; Sjogren's syndrome; Sleep Apnea; Soto's syndrome; Spasticity; Spina bifida; Spinal cord injury; Spinal cord tumors; Spinal Muscular Atrophy; Stiff-Person syndrome; Stroke; Sturge-Weber syndrome; Subacute Sclerosing Panencephalitis; Subcortical Arteriosclerotic Encephalopathy; Sydenham Chorea; Syncope; Syringomyelia; Tardive dyskinesia; Tay-Sachs disease; Temporal arteritis; Tethered Spinal Cord syndrome; Thomsen disease; Thoracic Outlet syndrome; Tic Douloureux; Todd's Paralysis; Tourette syndrome; Transient ischemic attack; Transmissible Spongiform Encephalopathies; Transverse myelitis; Traumatic Brain injury; Tremor; Trigeminal Neuralgia; Tropical Spastic Paraparesis; Tuberous Sclerosis; Vascular Dementia (Multi-Infarct Dementia); Vasculitis including Temporal Arteritis; Von Hippel-Lindau Disease (VHL); Wallenberg's syndrome; Werdnig-Hoffman disease; West syndrome; Whiplash; Williams syndrome; Wilson's disease; and Zellweger syndrome.

As used herein, “opthalmologic disease” or “opthalmologic disorder” refers to disease or disorder involving the anatomy and/or function of the visual system, including but not limited to, glaucoma, retinal artery occlusion, ischemic optic neuropathy and macular degeneration (wet or dry).

The neurological disorder can be an affective disorder (e.g., depression or anxiety). As used herein, “affective disorder” or “mood disorder” refers to a variety of conditions characterized by a disturbance in mood as the main feature. If mild and occasional, the feelings may be normal. If more severe, they may be a sign of a major depressive disorder or dysthymic reaction or be symptomatic of bipolar disorder. Other mood disorders may be caused by a general medical condition. See, Mosby's Medical, Nursing & Allied Health Dictionary, 5th Edition (1998).

As used herein, “depression” refers to an abnormal mood disturbance characterized by feelings of sadness, despair, and discouragement. Depression refers to an abnormal emotional state characterized by exaggerated feelings of sadness, melancholy, dejection, worthlessness, emptiness, and hopelessness, that are inappropriate and out of proportion to reality. See, Mosby's Medical, Nursing & Allied Health Dictionary, 5th Edition (1998). Depression can be at least one of a major depressive disorder (single episode, recurrent, mild, moderate, severe without psychotic features, severe with psychotic features, chronic, with catatonic features, with melancholic features, with atypical features, with postpartum onset, in partial remission, in full remission), dysthymic disorder, adjustment disorder with depressed mood, adjustment disorder with mixed anxiety and depressed mood, premenstrual dysphoric disorder, minor depressive disorder, recurrent brief depressive disorder, postpsychotic depressive disorder of schizophrenia, a major depressive disorder associated with Parkinson's disease, and a major depressive disorder associated with dementia.

The neurological disorder can be pain associated depression (PAD). As used herein, “pain associated depression” or “PAD” is intended to refer to a depressive disorder characterized by the co-morbidity of pain and atypical depression. Specifically, the pain can be chronic pain, neuropathic pain, or a combination thereof. Specifically, the pain associated depression (PAD) can include atypical depression and chronic pain wherein the chronic pain precedes the atypical depression. Alternatively, the pain associated depression (PAD) can include atypical depression and chronic pain wherein the atypical depression precedes the chronic pain. The pain associated depression (PAD) includes atypical depression and neuropathic pain.

“Chronic pain” refers to pain that continues or recurs over a prolonged period of time (i.e., >3 mos.), caused by various diseases or abnormal conditions, such as rheumatoid arthritis. Chronic pain may be less intense than acute pain. The person with chronic pain does not usually display increased pulse and rapid perspiration because the automatic reactions to pain cannot be sustained for long periods of time. Others with chronic pain may withdraw from the environment and concentrate solely on their affliction, totally ignoring their family, their friends, and external stimuli. See, Mosby's Medical, Nursing & Allied Health Dictionary, 5th Edition (1998).

Chronic pain can be selected from the group of lower back pain, atypical chest pain, headache, pelvic pain, myofascial face pain, abdominal pain, and neck pain or chronic pain is caused by a disease or condition selected from the group of arthritis, temporal mandibular joint dysfunction syndrome, traumatic spinal cord injury, multiple sclerosis, irritable bowel syndrome, chronic fatigue syndrome, premenstrual syndrome, multiple chemical sensitivity, closed head injury, fibromyalgia, rheumatoid arthritis, diabetes, cancer, HIV, interstitial cystitis, migraine headache, tension headache, post-herpetic neuralgia, peripheral nerve injury, causalgia, post-stroke syndrome, phantom limb syndrome, and chronic pelvic pain.

“Atypical depression” refers to a depressed affect, with the ability to feel better temporarily in response to positive life effect (mood reactivity), plus two or more neurovegetative symptoms selected from the group of hypersomnia, increased appetite or weight gain, leaden paralysis, and a long standing pattern of extreme sensitivity to perceived interpersonal rejection; wherein the neurovegetative symptoms are present for more than about two weeks. It is appreciated that those of skill in the art recognize that the neurovegatative symptoms can be reversed compared to those found in other depressive disorders (e.g., melancholic depression); hence the term “atypical.”

As used herein, “mammal” refers to a class of vertebrate animals of more than 15,000 species, including humans, distinguished by self-regulating body temperature, hair, and in the females, milk-producing mammae. Specifically, mammal can refer to a human. More specifically, mammal can refer to a human adult, e.g., 18 years or older. More specifically, mammal can refer to an elderly human adult, e.g., 60 years or older.

As used herein, “acute neurological disorder” refers to a neurological disorder, as defined above, wherein the disorder has a rapid onset which is followed by a short but severe course, including, but not limited to, Febrile Seizures, Guillain-Barre syndrome, stroke, and intracerebral hemorrhaging (ICH).

As used herein, “chronic neurological disorder” refers to a neurological disorder, as defined above, wherein the disorder lasts for a long period of time

(e.g., more than about 2 weeks; specifically, the chronic neurological disorder can continue or recur for more than about 4 weeks, more than about 8 weeks, or more than about 12 weeks) or is marked by frequent recurrence, including, but not limited to, narcolepsy, chronic inflammatory demyelinating polyneuropathy, Cerebral palsy (CP), epilepsy, multiple sclerosis, dyslexia, Alzheimer's disease and Parkinson's Disease.

As used herein, “trauma” refers to any injury or shock to the body, as from violence or an accident. The term trauma also refers to any emotional wound or shock, many of which may create substantial, lasting damage to the psychological development of a person, often leading to neurosis.

As used herein, “ischemic conditions” refers to any condition which results in a decrease in the blood supply to a bodily organ, tissue, or part caused by constriction or obstruction of the blood vessels, often resulting in a reduction of oxygen to the organ, tissue, or part.

As used herein, “hypoxic conditions” refers to conditions in which the amount/concentration of oxygen in the air, blood or tissue is low (subnormal).

As used herein, “painful neuropathy” or “neuropathy” refers to chronic pain that results from damage to or pathological changes of the peripheral or central nervous system. Peripheral neuropathic pain is also referred to as painful neuropathy, nerve pain, sensory peripheral neuropathy, or peripheral neuritis. With neuropathy, the pain is not a symptom of injury, but rather the pain is itself the disease process. Neuropathy is not associated with the healing process. Rather than communicating that there is an injury somewhere, the nerves themselves malfunction and become the cause of pain.

“Neuropathic pain” refers to pain associated with inflammation or degeneration of the peripheral nerves, cranial nerves, spinal nerves, or a combination thereof. The pain is typically sharp, stinging, or stabbing. The underlying disorder can result in the destruction of peripheral nerve tissue and can be accompanied by changes in the skin color, temperature, and edema. See, Mosby's Medical, Nursing & Allied Health Dictionary, 5th Edition (1998); and Stedman's Medical Dictionary, 25th Edition (1990).

As used herein, “diabetic neuropathy” refers to a peripheral nerve disorder/nerve damage caused by diabetes, including peripheral, autonomic, and cranial nerve disorders/damage associated with diabetes. Diabetic neuropathy refers to a common complication of diabetes mellitus in which nerves are damaged as a result of hyperglycemia (high blood sugar levels).

As used herein, “drug dependence” refers to habituation to, abuse of, and/or addiction to a chemical substance. Largely because of psychological craving, the life of the drug-dependent person revolves around the need for the specific effect of one or more chemical agents on mood or state of consciousness. The term thus includes not only the addiction (which emphasizes the physiological dependence) but also drug abuse (in which the pathological craving for drugs seems unrelated to physical dependence). Examples include, but are not limited to, alcohol, opiates, synthetic analgesics with morphine-like effects, barbiturates, hypnotics, sedatives, some antianxiety agents, cocaine, psychostimulants, marijuana, nicotine and psychotomimetic drugs.

As used herein, “drug withdrawal” refers to the termination of drug taking. Drug withdrawal also refers to the clinical syndrome of psychological, and, sometimes physical factors that result from the sustained use of a particular drug when the drug is abruptly withdrawn. Symptoms are variable but may include anxiety, nervousness, irritability, sweating, nausea, vomiting, rapid heart rate, rapid breathing, and seizures.

As used herein, “drug addiction” or dependence is defined as having one or more of the of the following signs: a tolerance for the drug (needing increased amounts to achieve the same effect), withdrawal symptoms, taking the drug in larger amounts than was intended or over a longer period of time than was intended, having a persistent desire to decrease or the inability to decrease the amount of the drug consumed, spending a great deal of time attempting to acquire the drug, or continuing to use the drug even though the person knows there are reoccurring physical or psychological problems being caused by the drug.

In one embodiment, when treating drug withdrawal, dependence and/or tolerance, the MMP inhibitor is administered with an NMDAR antagonist (e.g., memantine).

As used herein, “depression” refers to a mental state of depressed mood characterized by feelings of sadness, despair and discouragement. Depression ranges from normal feelings of the blues through dysthymia to major depression.

As used herein, “anxiety disorders” refers to an excessive or inappropriate aroused state characterized by feelings of apprehension, uncertainty, or fear. Anxiety disorders have been classified according to the severity and duration of their symptoms and specific behavioral characteristics. Categories include: Generalized anxiety disorder (GAD), which is long-lasting and low-grade; Panic disorder, which has more dramatic symptoms; Phobias; Obsessive-compulsive disorder (OCD); Post-traumatic stress disorder (PTSD); and Separation anxiety disorder.

As used herein, “tardive dyskinesia” refers to a serious, irreversible neurological disorder that can appear at any age. Tardive Dyskinesia, e.g., Tourette's syndrome, can be a side effect of long-term use of antipsychotic/neuroleptic drugs. Symptoms can be hardly noticeable or profound. Symptoms involve uncontrollable movement of various body parts, including the body trunk, legs, arms, fingers, mouth, lips, or tongue.

As used herein, “movement disorder” refers to a group of neurological disorders that involve the motor and movement systems, including, but are not limited to, Ataxia, Parkinson's disease, Blepharospasm, Angelman Syndrome, Ataxia Telangiectasia, Dysphonia, Dystonic disorders, Gait disorders, Torticollis, Writer's Cramp, Progressive Supranuclear Palsy, Huntington's Chorea, Wilson's Disease, Myoclonus, Spasticity, Tardive dyskinesia, Tics and Tourette syndrome and Tremors.

As used herein, “cerebral infections that disrupt the blood-brain barrier” refers to infections of the brain or cerebrum that result in an alteration in the effectiveness of the blood-brain barrier, either increasing or decreasing its ability to prevent, for example, substances and/or organisms from passing out of the bloodstream and into the CNS.

As used herein “the blood-brain barrier” refers to a semi-permeable cell layer of endothelial cells (interior walls) within capillaries of the central nervous system (CNS). The blood-brain barrier prevents large molecules, immune cells, many potentially damaging substances, and foreign organisms (e.g., viruses), from passing out of the bloodstream and into the CNS (Brain and Spinal Cord). A dysfunction in the Blood-Brain Barrier may underlie in part the disease process in MS (multiple sclerosis).

As used herein, “meningitis” refers to inflammation of the meninges of the brain and the spinal cord, most often caused by a bacterial or viral infection and characterized by fever, vomiting, intense headache, and stiff neck.

As used herein, “meningoencephalitis” refers to inflammation of both the brain and meninges.

As used herein, “stroke” refers to a sudden loss of brain function caused by a blockage or rupture of a blood vessel to the brain (resulting in the lack of oxygen to the brain), characterized by loss of muscular control, diminution or loss of sensation or consciousness, dizziness, slurred speech, or other symptoms that vary with the extent and severity of the damage to the brain. Also called cerebral accident, cerebrovascular accident.

As used herein, “hypoglycemia” refers to an abnormally low level of glucose in the blood.

As used herein, “cerebral ischemia (stroke)” refers to a deficiency in blood supply to the brain, often resulting in a lack of oxygen to the brain.

As used herein, “cardiac arrest” refers to a sudden cessation of heartbeat and cardiac function, resulting in a temporary or permanent loss of effective circulation.

As used herein, “spinal cord trauma” refers to damage to the spinal cord that results from direct injury to the spinal cord itself or indirectly by damage to the bones and soft tissues and vessels surrounding the spinal cord. It is also called Spinal cord compression; Spinal cord injury; or Compression of spinal cord.

As used herein, “head trauma” refers to ahead injury of the scalp, skull, or brain. These injuries can range from a minor bump on the skull to a devastating brain injury. Head trauma can be classified as either closed or penetrating. In a closed head injury, the head sustains a blunt force by striking against an object. A concussion is a type of closed head injury that involves the brain. In a penetrating head injury, an object breaks through the skull and enters the brain. (This object is usually moving at a high speed like a windshield or another part of a motor vehicle.)

As used herein, “perinatal hypoxia” refers to a lack of oxygen during the perinatal period (defined as the period of time occurring shortly before and after birth, variously defined as beginning with completion of the twentieth to twenty eighth week of gestation and ending 7 to 28 days after birth).

As used herein, “hypoglycemic neuronal damage” refers to neuronal damage, for example, nerve damage, as a result of a hypoglycemic condition (an abnormally low level of glucose in the blood).

As used herein, “neurodegenerative disorder” refers to a type of neurological disease marked by the loss of nerve cells, including, but not limited to, Alzheimer's disease, Parkinson's disease, amyotrophic lateral sclerosis, tauopathies (including fronto-temporal dementia), and Huntington's disease.

As used herein, “epilepsy” refers to any of various neurological disorders characterized by sudden recurring attacks of motor, sensory, or psychic malfunction with or without loss of consciousness or convulsive seizures.

As used herein, “Alzheimer's disease” refers to a disease marked by the loss of cognitive ability, generally over a period of 10 to 15 years, and associated with the development of abnormal tissues and protein deposits in the cerebral cortex (known as plaques and tangles).

As used herein, “Huntington's disease” refers to a disease that is hereditary in nature and develops in adulthood and ends in dementia. More specifically, Huntington's disease (HD) results from genetically programmed degeneration of brain cells, called neurons, in certain areas of the brain caused by a polyglutamine repeat in the DNA sequence of the gene encoding the protein huntingtin. This degeneration causes uncontrolled movements, loss of intellectual faculties, and emotional disturbance.

As used herein, “Parkinsonism” refers to a disorder similar to Parkinson's disease, but which is caused by the effects of a medication, a different neurodegenerative disorder or another illness. The term “parkinsonism” also refers to any condition that causes any combination of the types of movement abnormalities seen in Parkinson's disease by damaging or destroying dopamine neurons in a certain area of the brain.

As used herein, “amyotrophic lateral sclerosis” (ALS), also called Lou Gehrig's disease and Motor Neuron Disease, refers to a progressive, fatal neurological disease. The disorder belongs to a class of disorders known as motor neuron diseases. ALS occurs when specific nerve cells in the brain and spinal cord that control voluntary movement gradually degenerate (usually the “upper” (in the cerebrocortex) and “lower” (in the spinal cord) motor neurons, although some variants known as primary lateral sclerosis, apparently representing a separate disease, affect only the upper motor neurons). The loss of these motor neurons causes the muscles under their control to weaken and waste away, leading to paralysis. ALS manifests itself in different ways, depending on which muscles weaken first. Symptoms may include tripping and falling, loss of motor control in hands and arms, difficulty speaking, swallowing and/or breathing, persistent fatigue, and twitching and cramping, sometimes quite severely. Upper motor neuron variants (e.g., primary lateral sclerosis) are also included.

As used herein, “glaucoma” refers to any of a group of eye diseases characterized by abnormally high intraocular fluid pressure, damaged optic disk, hardening of the eyeball, and partial to complete loss of vision. The retinal ganglion cells are lost in glaucoma. Some variants of glaucoma have normal intraocular pressure (known also as low tension glaucoma).

As used herein, “retinal ischemia” refers to a decrease in the blood supply to the retina.

As used herein, “ischemic optic neuropathy” refers to a condition that usually presents with sudden onset of unilaterally reduced vision. The condition is the result of decreased blood flow to the optic nerve (ischemia). There are two basic types: arteritic and non-arteritic ischemic optic neuropathy. Non-arteritic ischemic optic neuropathy is generally the result of cardiovascular disease. Those patients at greatest risk have a history of high blood pressure, elevated cholesterol, smoking, diabetes, or combinations of these. Arteritic ischemic optic neuropathy is a condition caused by the inflammation of vessels supplying blood to the optic nerve, known as temporal arteritis. This condition usually presents with sudden and severe vision loss in one eye, pain in the jaw with chewing, tenderness in the temple area, loss of appetite, and a generalized feeling of fatigue or illness.

As used herein, “macular degeneration” refers to the physical disturbance of the center of the retina called the macula. The macula is the part of the retina which is capable of our most acute and detailed vision. Macular degeneration is the leading cause of legal blindness in people over age 55. (Legal blindness means that a person can see 20/200 or less with eyeglasses.) Even with a loss of central vision, however, color vision and peripheral vision may remain clear. Vision loss usually occurs gradually and typically affects both eyes at different rates.

As used herein a “demyelinating disorder” refers to a medical condition where the myelin sheath is damaged. The myelin sheath surrounds nerves and is responsible for the transmission of impulses to the brain. Damage to the myelin sheath may result in muscle weakness, poor coordination and possible paralysis. Examples of demyelinating disorders include Multiple Sclerosis (MS), optic neuritis, transverse neuritis and Guillain-Barre Syndrome (GBS). In one embodiment, when treating a demyelinating disorder, an MMP inhibitor is administered with an NMDAR antagonist (e.g., memantine) or with (J-interferon isoforms, copaxone or Antegren (natalizumab)). Recently, it has been noted that underlying neuronal damage can occur in demyelinating conditions such as MS, and therefore useful drugs may also protect the neurons instead or in addition to the myelin.

As used herein, “multiple sclerosis” refers to a chronic disease of the central nervous system, which predominantly affects young adults. Viral and autoimmune etiologies are postulated. Genetic and environmental factors are known to contribute to MS, but a specific cause for this disease is not yet identified. Pathologically, MS is characterized by the presence of areas of demyelination and T-cell predominant perivascular inflammation in the brain white matter. Some axons may be spared from these pathological processes. The disease begins most commonly with acute or subacute onset of neurologic abnormalities. Initial and subsequent symptoms may dramatically vary in their expression and severity over the course of the disease, that usually lasts for many years. Early symptoms may include numbness and/or paresthesia, mono- or paraparesis, double vision, optic neuritis, ataxia, and bladder control problems. Subsequent symptoms also include more prominent upper motor neuron signs, i.e., increased spasticity, increasing para- or quadriparesis. Vertigo, incoordination and other cerebellar problems, depression, emotional lability, abnormalities in gait, dysarthria, fatigue and pain are also commonly seen.

As used herein, “sequelae of hyperhomocysrinemia” refers to a condition following as a consequence hyperhomocystinemia, meaning elevated levels of homocysteine.

As used herein, “convulsion” refers to a violent involuntary contraction or series of contractions of the muscles.

As used herein, “pain” refers to an unpleasant sensation associated with actual or potential tissue damage, and mediated by specific nerve fibers to the brain where its conscious appreciation may be modified by various factors. See, Mosb's Medical, Nursing & Allied Health Dictionary, 5th Edition (1998); and Stedman's Medical Dictionary, 25th Edition (1990).

As used herein, “anxiety” refers to a state of apprehension, uncertainty, and/or fear resulting from the anticipation of a realistic or fantasized threatening event or situation, often impairing physical and psychological functioning.

As used herein, “schizophrenia” refers to any of a group of psychotic disorders usually characterized by withdrawal from reality, illogical patterns of thinking, delusions, and hallucinations, and accompanied in varying degrees by other emotional, behavioral, or intellectual disturbances. Schizophrenia is associated with dopamine imbalances in the brain and defects of the frontal lobe and is caused by genetic, other biological, and/or psychosocial factors.

As used herein, “muscle spasm” refers to an often painful involuntary muscular contraction

As used herein, “migraine headache” refers to a severe, debilitating headache often associated with photophobia and blurred vision.

As used herein, “urinary incontinence” refers to the inability to control the flow of urine and involuntary urination.

As used herein, “nicotine withdrawal” refers to the withdrawal from nicotine, an addictive drug found in tobacco, which is characterized by symptoms that include headache, anxiety, nausea and a craving for more tobacco. Nicotine creates a chemical dependency, so that the body develops a need for a certain level of nicotine at all times. Unless that level is maintained, the body will begin to go through withdrawal. For tobacco users trying to quit, symptoms of withdrawal from nicotine are unpleasant and stressful, but temporary. Most withdrawal symptoms peak 48 hours after one quits and are completely gone in six months.

As used herein, “opiate tolerance” can be explained, at least in part, as a homeostatic response that reduces the sensitivity of the system to compensate for continued exposure to high levels of, for example, morphine or heroin. When the drug is stopped, the system is no longer as sensitive to the soothing effects of the enkephalin neurons and the pain of withdrawal is produced.

As used herein, “opiate withdrawal” refers to an acute state caused by cessation or dramatic reduction of use of opiate drugs that has been heavy and prolonged (several weeks or longer). Opiates include heroin, morphine, codeine, Oxycontin, Dilaudid, methadone, and others. The reaction frequently includes sweating, shaking, headache, drug craving, nausea, vomiting, abdominal cramping, diarrhea, inability to sleep, confusion, agitation, depression, anxiety, and other behavioral changes.

As used herein, “emesis” refers to the act of vomiting.

As used herein, “brain edema” refers to an excessive accumulation of fluid in, on, around and/or in relation to the brain.

As used herein, “AIDS induced dementia” or “HIV-associated dementia” refers to dementia (deterioration of intellectual faculties, such as memory, concentration, and judgment, resulting from an organic disease or a disorder of the brain) induced by AIDS (Acquired Immunodeficiency Syndrome—an epidemic disease caused by an infection by human immunodeficiency virus (HIV-1, HIV-2), a retrovirus that causes immune system failure and debilitation and is often accompanied by infections such as tuberculosis).

As used herein, “HIV-related neuropathy” refers to a neuropathy in a mammal infected with HIV were the neuropathy is caused by infections such as CMV or other viruses of the herpes family. Neuropathy is the name given to a group of disorders whose symptoms may range from a tingling sensation or numbness in the toes and fingers to paralysis. Neuropathy might more accurately be called “neuropathies” because there are several types and can be painful.

As used herein, “ocular damage” refers to any damage to the eyes or in relation to the eyes.

As used herein, “retinopathy” refers to any pathological disorder of the retina.

As used herein, “cognitive disorder” refers to any cognitive dysfunction, for example, disturbance of memory (e.g., amnesia) or learning.

As used herein, “neuronal injury associated with HIV infection” refers to damage/injury of nerve cells caused either directly or indirectly by infection with HIV.

As used herein, “dysfunction in cognition, movement and sensation” refers to abnormal or impaired functioning in cognition (mental process of knowing, including aspects such as awareness, perception, reasoning, and judgment), movement or sensation.

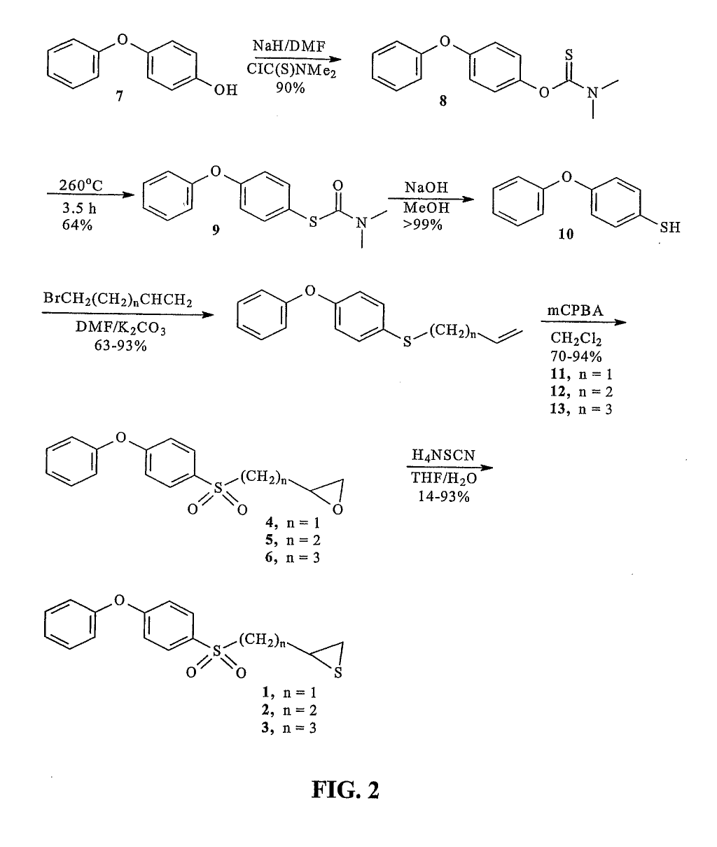

FIG. 2 illustrates a synthesis for compounds 1-3. 4-phenoxythiophenol 10 was prepared from the commercially available 4-phenoxyphenol 7 via the 3 step procedure illustrated by Newman and Karnes. Newman M. S.; Karnes H. A. J. Org. Chem., 1996, 31, 3980-3984. Subsequent alkylation of 10 with allyl bromide, 4-bromo-1-butene and 5-bromo-1-pentene respectively, led to the sulfanyl compounds 11-13 in good yield. Although the epoxidation of 12 and 13 with mCPBA was relatively quick, taking only 2-3 days, the formation of 11 took 7 days and required a large excess of mCPBA. Finally, the conversion of the epoxides 4-6 to their corresponding thiirane derivatives 1-3, was accomplished via the treatment of each epoxide with ammoniumthiocyanate in THF/water. Although the thiiranes 2 and 3 were isolated in high yield, 93% and 85% respectively, thiirane 1 could only be recovered in a very poor (i.e., 14%) yield.

Processes for preparing compounds of formula (I) or for preparing intermediates useful for preparing compounds of formula (I) are provided as further embodiments of the invention. Intermediates useful for preparing compounds of formula (I) are also provided as further embodiments of the invention.

A compound of formula (I) wherein J is S can be prepared by treating a corresponding compound of formula (I) wherein J is O with a suitable sulfonating reagent. See, e.g., March, Advanced Organic Chemistry, Reactions, Mechanisms and Structure, 2^{nd} Ed., 1977 and Carey & Sundberg, Advanced Organic Chemistry, Part B: Reactions, 2^{nd} Ed., 1983.

A compound of formula (I) wherein J is O can be prepared by epoxidizing a corresponding compound of formula (I) wherein the ring that includes J is an alkene. See, e.g., March, Advanced Organic Chemistry, Reactions, Mechanisms and Structure, 2^{nd} Ed., 1977 and Carey & Sundberg, Advanced Organic Chemistry, Part B: Reactions, 2^{nd} Ed., 1983.

A compound of formula (I) wherein D is SO₂ and J is O can be prepared by oxidizing a corresponding compound of formula (I) wherein D is S. See, e.g., March, Advanced Organic Chemistry, Reactions, Mechanisms and Structure, 2^{nd} Ed., 1977 and Carey & Sundberg, Advanced Organic Chemistry, Part B: Reactions, 2^{nd} Ed., 1983.

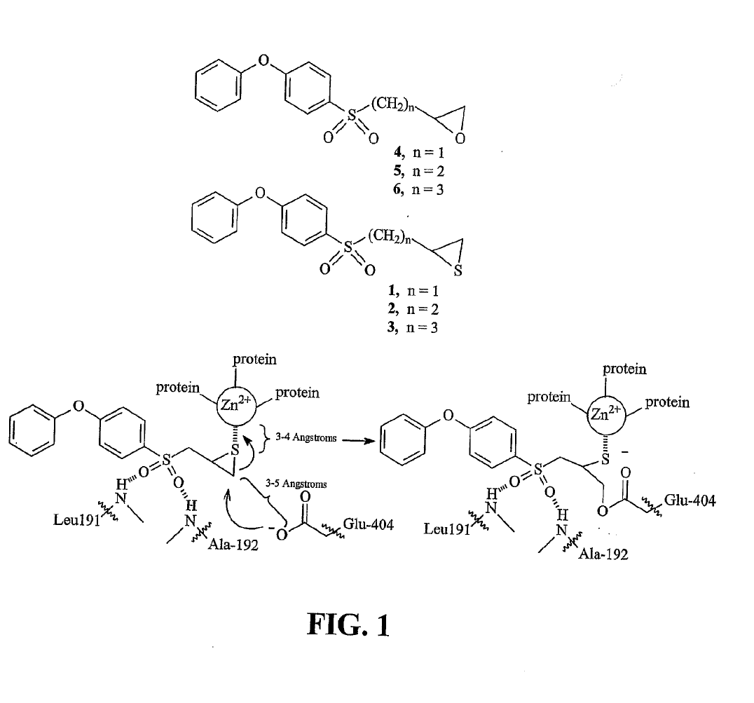

A specific group of the compounds of the present invention, that can be activated by zinc for nucleophilic substitution and that can form a covalent bond with a nucleophile of the matrix metalloproteinase, includes a thiirane ring. Another specific group of the compounds of the present invention, that can be activated by zinc for nucleophilic substitution and that can form a covalent bond with a nucleophile of the matrix metalloproteinase, includes an oxirane ring. In addition, a specific nucleophile of the matrix metalloproteinase which can form a covalent bond with the group of the compounds of the present invention (e.g., thiirane or oxirane) is located at the amino acid residue corresponding to residue 404 of the matrix metalloproteinase, wherein the numbering is based on the active site general base for gelatinase A, which is observed in other MMPs. More specifically, the nucleophile is a carboxy (i.e., COO⁻) oxygen atom located at amino acid residue corresponding to residue 404 of the matrix metalloproteinase, wherein the numbering is based on the active site general base for gelatinase A, which is observed in other MMPs. See, FIG. 1.

The matrix metalloproteinase can be a human matrix metalloproteinase. In addition, the matrix metalloproteinase can be a gelatinase, collagenase, stromelysin, membrane-type MMP, or matrilysin. Specifically, the gelatinase can be MMP-2 or MMP-9.

According to the method of the invention, the matrix metalloproteinase can be contacted with the compound, e.g., a compound of formula (I), in vitro. Alternatively, the matrix metalloproteinase can be contacted with the compound, e.g., a compound of formula (I), in vivo.

Without being bound by any particular theory, coordination of a thiirane in a compound of formula (I) with the enzyme active-site zinc ion is believed to activate the thiirane for modification by a nucleophile of the enzyme. See, FIG. 1. A computational model based on three-dimensional homology modeling for this enzyme with compound 1 indicates that the biphenyl group would fit in the active site analogously to the same group in certain known reversible inhibitors of MMP-2 and MMP-9, as analyzed by X-ray structure determination. Freskos, J. N.; Mischke B. V.; DeCrescenzo, G. A.; Heintz, R.; Getman, D. P.; Howard, S. C; Kishore, N. N.; McDonald, J. J.; Munie, G. E.; Rangwala, S.; Swearingen, C. A.; Voliva, C; Welsch, D. J. Bioorg. & Med. Chem. Letters, 1999, 9, 943-948. Tamura, Y.; Watanabe, F.; Nakatani, T.; Yasui, K.; Fuji, M.; Komurasaki, T.; Tsuzuki, H.; Maekawa, R.; Yoshioka, T.; Kawada, K.; Sugita, K.; Ohtani, M. J. Med. Chem. 1998, 41, 640-649. As such, the biphenyl ether moiety in compounds 1-3 is believed to fit in the P1′ subsite of gelatinases, which is a deep hydrophobic pocket, (a) Morgunova, E.; Tuuttila, A.; Bergmann, U.; Isupov, M.; Lindqvist, Y.; Schneider, G.; Tryggvason, K. Science 1999, 284, 1667-1670. (b) Massova, I.; Fridman, R.; Mobashery, S. J. Mol. Mod. 1997, 3, 17-34; Olson, M, W.; Bernardo, M. M.; Pietila, M.; Gervasi, D. C.; Toth, M.; Kotra, L. P.; Massova, L; Mobashery, S.; Fridman, R. J. Biol. Chem., 2000, 275, 2661-2668. This binding mode brings the sulfur of the thiirane in 1 into the coordination sphere of the zinc ion. See, FIG. 1. The models also indicated that the thiirane moiety in compounds 2 and 3, with longer carbon backbones, would not be able to coordinate with the zinc ion as well as compound 1, but would fit in an extended configuration in the active site.

It is believed that the high specificity of certain compounds of the invention for a targeted enzyme arises predominantly from three factors, (i) the compounds satisfy the binding specificity requirements at the active site. In this respect these compounds are not any different from conventional reversible or affinity inhibitors, (ii) Furthermore, the structural features of the inhibition should allow it to undergo chemical activation by the zinc atom of the enzyme to generate an electrophilic species within the active site, (iii) Finally, there should be a nucleophilic amino-acid residue in the active site, in the proper orientation, to react with the electrophilic species (e.g., thiirane ring), resulting in irreversible enzyme inactivation.

By selecting a hydrophobic group (e.g., A-X-M) located a specific distance from a group (e.g., D) that can bind (e.g., hydrogen bond) with one or more sites in the enzyme (e.g., amino acid residue 191 and/or amino acid residue 192, in gelatinase A), which is in turn located a specific distance from a thiirane ring that can coordinate with the enzyme active-site zinc atom, one can prepare selective mechanism-based inhibitors for a given MMP. See, FIG. 1.

Accordingly, preferred MMP inhibitors have a hydrophobic aryl moiety (e.g., A-X-M) that can fit in the deep hydrophobic pocket (i.e., P₁′ subsite) of an MMP. In addition, preferred mechanism-based MMP inhibitors also have a thiirane ring that can coordinate with the enzyme active-site zinc ion, and be modified by a nucleophile (e.g., carboxylate group of amino acid residue 404 of MMP-2) in the enzyme active site. See, FIG. 1. The preferred MMP inhibitors can optionally include a second group (e.g., D) that can coordinate with one or more sites in the enzyme. Specifically, the second group can optionally hydrogen bond to the one or two proton donors (e.g., amino acid residue corresponding to residue 191 and/or amino acid residue corresponding to residue 192 of MMP-2) in the enzyme active site. See, FIG. 1.

The present invention provides a method for identifying a mechanistic based MMP inhibitor. The method includes providing a compound wherein (1) a hydrophobic moiety of the compound fits into a hydrophobic pocket of the MMP; (2) the compound has one or two groups that can hydrogen bond with one or two hydrogen donors of the MMP, wherein the hydrogen donors of the MMP are located at amino acid residue corresponding to residue 191 and amino acid residue corresponding to residue 192 of MMP-2; (3) the compound has an electrophilic group that can covalently bond with a nucleophile of the MMP, wherein the nucleophile of the MMP is located at amino acid residue corresponding to residue 404 of MMP-2; and/or (4) the compound includes a group that can coordinate with the zinc ion of the MMP.

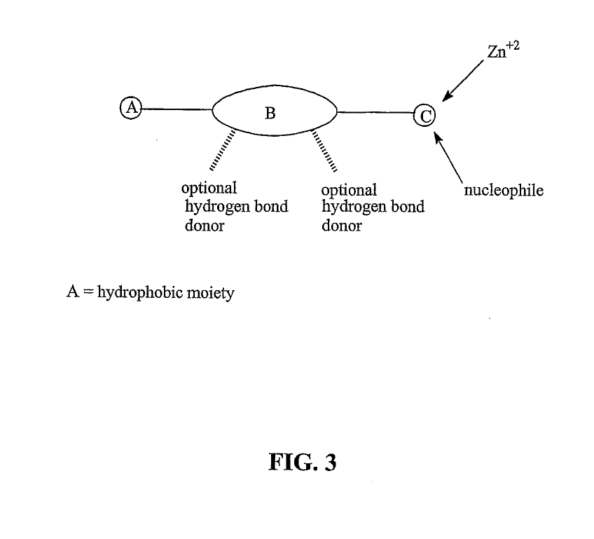

Preferred MMP inhibitors have a thiirane or oxirane such that the sulfur or oxygen atom of the thiirane or oxirane is located about 3 angstroms to about 4 angstroms from the zinc ion. The suitable MMP inhibitors can also include a thiirane or oxirane ring located about 3 angstroms to about 5 angstroms from the active site nucleophile. See, FIGS. 1 and 3.

A compound of formula (I), or a pharmaceutically acceptable salt thereof, can be administered to a mammal (e.g., human) in conjunction with a neurological agent, or a pharmaceutically acceptable salt thereof. Accordingly, a compound of formula (I) can be administered in conjunction with a neurological agent to treat a neurological disorder and/or an opthalmological disorder.

As used herein, a “neurological agent” is a compound, including chemical and biological compounds (e.g., peptides, oligonucleotides and antibodies), that has an affect on the nervous system, e.g., compounds capable of treating, inhibiting or preventing disorders affecting the nervous system or compounds capable of eliciting a neurological and/or an opthalmological disorder or symptoms thereof.

### Combination of Components (a) and (b)

In the following description component (b) is to be understood to represent one or more agents as described previously (e.g., a compound of formula (I)). Thus, if components (a) and (b) are to be treated the same or independently, each agent of component (b) may also be treated the same or independently. Components (a) and (b) of the present invention may be formulated together, in a single dosage unit (that is, combined together, e.g., in one lotion, cream, gel or ointment) as a combination product. When component (a) and (b) are not formulated together in a single dosage unit, the component (a) may be administered at the same time as component (b) or in any order; for example component (a) of this invention may be administered first, followed by administration of component (b), or they may be administered in the reverse order. If component (b) contains more than one agent, e.g., antiviral agent and NSAID, these agents may be administered together or separately in any order. When not administered at the same time, preferably the administration of component (a) and (b) occurs less than about one hour apart.

As is appreciated by a medical practitioner skilled in the art, the dosage of the combination therapy of the invention may vary depending upon various factors such as the pharmacodynamic characteristics of the particular agent and its mode of administration, the age, health and weight of the recipient, the nature and extent of the symptoms, the kind of concurrent treatment, the frequency of treatment, and the effect desired, as described above. The proper dosage of components (a) and (b) of the present invention will be readily ascertainable by a medical practitioner skilled in the art, based upon the present disclosure. By way of general guidance, typically a daily dosage may be about 100 milligrams to about 1.5 grams of each component. If component (b) represents more than one compound, then typically a daily dosage may be about 100 milligrams to about 1.5 grams of each agent of component (b). Byway of general guidance, when the compounds of component (a) and component (b) are administered in combination, the dosage amount of each component may be reduced by about 70-80% relative to the usual dosage of the component when it is administered alone as a single agent for the treatment of a disorder, and related symptoms, in view of the synergistic effect of the combination.

Pharmaceutical kits useful for the treatment of disorders described herein, and related symptoms, which include a therapeutically effective amount of a pharmaceutical composition that includes a compound of component (a) and one or more compounds of component (b), in one or more sterile containers, are also within the ambit of the present invention. Sterilization of the container may be carried out using conventional sterilization methodology well known to those skilled in the art. Component (a) and component (b) may be in the same sterile container or in separate sterile containers. The sterile containers of materials may include separate containers, or one or more multi-part containers, as desired-Component (a) and component (b), may be separate, or physically combined into a single dosage form or unit as described above. Such kits may further include, if desired, one or more of various conventional pharmaceutical kit components, such as for example, one or more pharmaceutically acceptable carriers, additional vials for mixing the components, etc., as will be readily apparent to those skilled in the art. Instructions, either as inserts or as labels, indicating quantities of the components to be administered, guidelines for administration, and/or guidelines for mixing the components, may also be included in the kit.

The MMP inhibitor can optionally be co-administered with a neuroprotectant drug, used, for example, in the treatment of Alzheimer's disease or other neurologic or opthalmologic disorders (e.g., glaucoma), including, but not limited to, memantine or a derivative thereof.

The MMP inhibitor can optionally be co-administered with at least one of the following:

An anti glaucoma agent, beta adrenergic blocking agent, carbonic

anhydrase inhibitor, miotic agent, sympathomimetic agent, acetylcholine blocking agent, antihistamine, anti-viral agent, quinolone, anti-inflammatory agent, non-steroidal anti-inflammatory agent, steroidal anti-inflammatory agent, antidepressant (e.g., serotonin reuptake inhibitors, SSRIs), psychotherapeutic agent, anti-anxiety agent, analgesic, antiseizure agent, anti-convulsant, gabapentine, anti-hypertensive agent, benzoporphyrin photosensitiser, immunosuppressive antimetabolite, anti-convulsant, barbiturate, benzodiazipine, GABA inhibitors, hydantoin, anti-psychotic, neurolaptic, antidysknetic, adrenergic agent, tricyclic antidepressant, anti-hypoglycemic, glucose solution, plypeptide hormone, antibiotic, thrombolytic agent, blood thinner, antiarrhythmic agent, corticosteroid, seizure disorder agent, anticholinesterase, dopamine blocker, antiparkinsonian agent, muscle relaxant, anxiolytic muscle relaxant, CNS stimulant, antiemetic, beta adrenergic blocking agents, ergot derivative, isometheptene, antiserotonin agent, analgesic, selective serotonin reuptake inhibitors (SSRIs), monoamine oxidase inhibitor, aids adjunct agents, anti infective agent, systemic aids adjunct anti infective, aids chemotherapeutic agent, nucleoside reverse transcriptase, and a protease inhibitor.

Specifically, the MMP inhibitor can optionally be co-administered with at least one of the following:

A beta adrenergic blocking agent, carbonic anhydrase inhibitor, cholinesterase inhibitor, cholinergic (miotic), docosanoid, prostaglandin, tricyclic antidepressant, psychotherapeutic agent, antianxiety agent, analgesic, anti-seizure agent, tricyclic antidepressants having analgesic effect in neuropathic pain, linolinic acid, coenzyme, vitamin, immunosuppressive antimetabolite, antiviral, copolymer, barbiturate, benzodiazepine, GABA inhibitor, hydantoin, tranquilizer, anti-psychotic, norephedrine, peptide, antibacterial, tissue plasminogen activator (TPA), blood thinner/anticoagulant, cardiostimulant, carbonic anhydrase inhibitor, ketoderivative of carbamazepine, acetylcholinesterase, antipsychotic, alkaloid, GABA-B receptor agonist, benzodiazepine, antiparkinsonian, antidepressant, CNS stimulant, receptor antagonist, beta adrenergic blocking agent, ergot derivatives (anti migraine), anticonvulsant, serotonin(5-HT) receptor agonist, antimanic, SSRI, MAOI, aids adjunct anti infective agent, antiviral, and protease inhibitor.

More specifically, the MMP inhibitor can optionally be co-administered with at least one of the following:

Timolol or Maleate, which is chemically designated as (2S)-1-[1,1-Dimethyl ethylamino]-3-[{4-(4-morpholinyl)-1,2,5-thiadiazole-3-yl}oxy]-2-propanol;

Betaxolol HCl, which is chemically designated as 1-[4 [2(Cyclopropyl methoxy)ethyl]-phenoxy]-3[(1-methylethyl)amino]-2-propanol;

Carteolol HCl, which is chemically designated as 5-[3-[(1,1Dimethylethyl) amino]-2hydroxypropoxy]-3,4-dihydro-2(1H)-quinolinone;

Metipranolol, which is chemically designated as 4-[2-Hydroxy-3-[(1-methylethyl)amino]propoxy]-2,3,6-trimethylphenol,1-acetate;

Timolol Hemihydrate, which is chemically designated as (2S)-1-[(1,1-Dimethylethyl)amino]-3-[{4-(4-morpholinyl)-1,2,5-thiadiazol-3-yl]oxy]-2-propanol;

Brimonidine Tartarate, which is chemically designated as 5-Bromo-N-4,5-dihydro-1H-imidazole-2-yl)-6-quinoxalmamine;

Brinzolamide, which is chemically designated as (4R)-4-(Ethylamino)-3,4-dihydro-2-(3-methoxypropyl)-2H-thieno[3,2-e]-1,2-thiazine-6-sulphonamide 1,1-dioxide;

Dorzolamide, which is chemically designated as (4S,6S)-4-(Ethylamino)-5,6-dihydro-6-methyl-4H-thieno[2,3-b]thiopyron-2-sulfonamide 7,7-dioxide;

Acetazolamide, which is chemically designated as N-[5-(Aminosulfonil)-1,3,4-thiadiazol-2-yl]acetamide; 5-acetamido-1,3,4-thiadiazole-2-sulfonamide;

Echothiophate Iodide, which is chemically designated as 2-[(Diethoxy-phosphinyl)thio]-N,N,N-trimethylethananaminium iodide;

Pilocarpine HCl, which is chemically designated as (3S-cis)-3-Ethyldihydro-4-[(1-methyl-1H-imidazol-5-yl)methyl]-2(3H)-furanone;

Unoprostone Isopropyl ester, which is chemically designated as (5Z)-7-[(1R,2R,3R-5S)-3,5-Dihydroxy-2-(3-oxodecyl)cyclopentyl]-5-heptinoic acid;

Latanoprost, which is chemically designated as 13,14-dihydro-17-phenyl-18,19,20-trinor-PGF2alpha-isopropyl ester;

Acamprosate, a drug with additional neuroprotective properties;

Amitriptyline, which is chemically designated as 3-(10 μl 1-Dihydro-5H-dibenzo[a,d]cyclohepten-5-ylidene)-N,N-dimethyl-1-propanamine;

Perphenazine, which is chemically designated as 4-[3-(2-Chloro-10H-phenothiazine-10-yl)propyl]-1-piperazineethanol;

Chlordiazepoxide, which is chemically designated as 7-Chloro-N-methyl-5-phenyl-3H-1,4-benzodiazepin-2-amine 4-oxide;

Trimipramine Maleate, which is chemically designated as 10,11-Dihydro-N,N,beta trimethyl-5H-dibenz[b,f]azepine-5-propanamine;

Chlodiazepoxide HCl, which is chemically designated as 7-Chloro-N-methyl-5-phenyl-3H-1,4-benzodiazepin-2-amine 4-oxide;

Alprazolam, which is chemically designated as 8-Chloro-1-methyl-6-phenyl-4H-[1,2,4]triazolo[4,3-a][1,4]benzodiazepine;

Hydroxyzine Di Hydrochloride, which is chemically designated as 2-[2-[4-[(4-Chlorophenyl)phenylmethyl]-1-piperazinyl]ethoxy, ethanol;

Meprobamate, which is chemically designated as (3S-trans)-3-[(1,3-banzodioxol-5-yloxy)methyl]-4-(4-fluorophenyl)piperidine;

Doxipin HCl, which is chemically designated as 3-Dibenz[b,e]oxepin-11-(6H)-ylidene-N,N-dimethyl-1-propanamine;

Hydroxyzine Pamoate, which is chemically designated as 2-[2-[4-[(4-Chlorophenyl)phenylmethyl]-1-piperazinyl]ethoxy,ethanol;

Aspirin, which is chemically designated as 2-(Acetyloxy)benzoicAcid;

Acetaminophen, which is chemically designated as N-(4-Hydroxyphenyl) acitamide;

Ibuprofen, which is chemically designated as Alpha-methyl-4-(2-methylpropyl)benzeneacetic acid;

Carbamazipine, which is chemically designated as 5H-Dibenz [b,f]azepine-5-carboxamide;

Flupirtine, a drug with neuroprotective properties using additional pathways to MMP antagonists, which is chemically designated as 2-amino-3emoxy-cabonoylammo-6-4-fluoro-benzylamino-pyridine malate;

Lamotrigine, which is chemically designated as 6-(2,3-Dichlorophenyl)-1,2,4-triazine-3,5-diamine;

Phenyloin Sodium, which is chemically designated as 5,5-Diphenyl-2,4-imidazolidinedione;

Pentaxifylline, which is chemically designated as 3,7-Dihydro-3,7-dimethyl-1-(5-oxohexyl)theobromine;

Thioctic Acid, which is chemically designated as 1,2-Dithiolane-3-pentanoic acid;

Levocarnitine, which is chemically designated as 3-carboxy-2-hydroxy-N,N,N-trimethyl-1-propanaminium;

Biotin, which is chemically designated as Hexahydro-2-oxo-1H-thieno[3,4-d]imidazoline-4-veleric acid;

Nicotinic acid, which is chemically designated as 3-pyridinecarboxylic acid;

Taurine, which is chemically designated as 2-Aminoethanesulfonic acid;

Verteporfin, which is chemically designated as (4R,4aS)-rel-18-Ethenyl-4,4a-dihydro-3,4-bis(methoxycarbonyl)-4a,8,14,19-tetramethyl-23H,25H-benzo[b]porphine-9,13-dipropanoic acid monomethyl ester;

Azathioprine, which is chemically designated as 6-[(1-Methyl-4-Nitro-1H-imidazol-5-yl)thio]-1H-purine; 6-(1-methyl-4-nitro-5-imidazolyl)mercaptopurine;

Interferon Beta 1b, which is a Glycoprotein containing 166 amino acids;

Interferon Beta 1a, which is a Glycoprotein containing 166 amino acids;

Cyclophosphamide, which is chemically designated as N,N-Bis(2-chloroethyl)tetrahydro-2H-1,3,2-oxazaphosphorin-2-amine-2-oxide monohydrate;

Methotrexate, which is chemically designated as N-[4-[{(2,4-Diamino-6-pteridmyl)methyl]methylamino]benzoyl]-L-glutamic acid;

Neurmexane, an NMDAR antagonist with reportedly improved properties to memantine;

Glatiramer, which is chemically designated as L-Glutamic Acid Polymer with L-alanine, L-lycine, and L-tyrocine;

Mephobarbitol, which is chemically designated as 5-Ethyl-1-methyl-5-phenyl-2,4,6(1H,3H,5H)-pyrimidinetrione;

Pentobarbitol, which is chemically designated as 5-Ethyl-5-(1-methylbutyl)-2,4,6(1H,3H,5H)-pyrimidinetrione; 5-ethyl-5-(1-methylbutyl)barbituric acid;

Lorazipam, which is chemically designated as 7-Chloro-5-(2-chlorophenyl)-1,3-dihydro-3-hydroxy-2H-1,4-benzodiazepin-2-one;

Clonazepam, which is chemically designated as 5-(2-Chlorophenyl)-1,3-dihydro-7-nitro-2H-1,4-benzodiazepin-2-one;

Chlorazeptate Dipotassium salt, which is chemically designated as 7-Chloro-2,3-dihydro-2-oxo-5-phenyl-1H-1,4-benzodiazepine-3-carboxylic acid monopotassium salt compound with potassium hydroxide;

Fosphenytoin Sodium, which is chemically designated as 5,5-Diphenyl-3-[(phosphonooxy)methyl]-2,4imidazolidinedione;

Olanzapine, which is chemically designated as 2-Methyl-4-(4-methyl-1-piperazinyl)-10H-thieno[2,3-b][1,5]benzodiazepine;

Heloperidol, which is chemically designated as 4-[4-(4-chlorophenyl)-4-hydroxy-1-piperidinyl]-1-(4-fluorophenyl)-1-butanone;

Trifluoperizine, which is chemically designated as 10-[3-(4-Methyl-1-piperazinyl)propyl]-2(trifluoromethyl)-10H-phenothiazine;

Fluphenazine, which is chemically designated as 4-[3-[2-(Trifluoromethyl)-10H-phenothiazin 10-yl]propyl]-1-piperazineethanol;

Phenylpropanol amine, which is chemically designated as (1RS,2SR)-2-amino-1-phenyl-1-propanol;

Pseudoephedrine HCl, which is chemically designated as (1S,2S)-2-methylamino-1-phenylpropan-1-ol;

Imipramine, which is chemically designated as 5-(3-dimethylaminopropyl)-10,11-dihydro-5H-dibenz[b,f]azepine;

Glucagon;

Glucagon-related peptide-1, which is identified as a 37 amino acid peptide;

Glucagon-related peptide-2, which is identified as a peptide that contains 33 amino acids;

Penicilin G, N, O, or V, which is chemically designated as (2S,5R,6R)-3,3-Dimethyl-7-oxo-6-[(phenylacetyl)amino]-4-thia-1-azabicyclo-[3.2.0]heptane-2-carboxylic acid;

Ampicillin, which is chemically designated as (2S,5R,6R)-6-[[(2R)-Aminophenylacetyl]amino]-3,3-dimethyl-7-oxo-4-thia-1-azabicyclo-[3,2,o]heptane-2-carboxylic acid;

Chloramphenicol, which is chemically designated as 2,2-Dichloro-N-[(1R,2R)-2-hydroxy-1-(hydroxymethyl)-2-(4-nitrophenyl)ethyl]acetamide;

Phorbol, which, is chemically designated as [1aR-(1aalpha,1bbeta,4abeta,7aalpha,7balpha,8alpha,9beta,9aalpha)]-1,1a,1b,4,4 a,7a,7b,8,9,9a-Decahyto-4a,-7b,9,9a-tetrahydroxy-3-(hydroxymethyl)-1,1,6,8-tetramethyl-5H-cyclopropa[3,4]benz[1,2-e]azulen-5-one;

Heparin, which is D-glucosamine with L-iduronic or D-glucuronic acids;

Warfarin, which is chemically designated as 4-Hydroxy-3-(3-0×0-1-phenyl-butyl)-2H-1benzopyran-2-one;

Epinephrine, which is chemically designated as 4-[(1R)-1-Hydroxy-2-(methylamino)ethyl]-1,2-benzenediol;

Amiodarone, which is chemically designated as (2-Butyl-3-benzofuranyl) [4-[2-(diethylamino)ethoxy]-3,5-diiodophenyl]methanone;

Lidocaine, which is chemically designated as 2-(Diethylamino)-N-(2,6-dimethylphenyl)acetamide;

Nitroglycerin, isosorbide dinitrate, amyl, butyl, isobutyl and various other nitrates have been shown to be neuroprotective;

Atenolol, which is chemically designated as 4-[2-Hydroxy-3-[(1-methylethyl)amino]propoxy]benzeneacetamide;

Dexamethasone, which is chemically designated as (11beta,16alpha)-9-Fluoro-11,17,21-trihydroxy-16-methylpregna-1,4-diene-3,20-dione;

Prednisolone, which is chemically designated as 1,4-pregnadiene-3,20-dione-11beta,17alpha,21-triol;

Acetazolamide, which is chemically designated as 2-acetylamino-1,3,4,-thiadiazole-5-sulfonamide;

Phenyloin, which is chemically designated as 5,5-Diphenyl-2,4-imidazolidinedione;

Tiagabin HCl, which is chemically designated as (3R)-1-[4,4-Bis(3-methyl-2-thienyl)-3-butenyl]-3-piperidinecarboxylic acid;

Gabapentin, which is chemically designated as 1-(Aminomethyl)-cyclohexaneacitic acid;

Oxacarbazepine, which is chemically designated as 10,11-Dihydro-10-oxo-5H-dibenz[b,f]azepine-5-carboxamide;

Tacrine, which is chemically designated as 1,2,3,4,-Tetrahydro-9-acridinamine;

Donepezil, which is chemically designated as 2,3-Dihydro-5,6-dimethoxy-2-[[1-(phenylmethyl)-4-piperidinyl]methyl]-1H-inden-1-one;

Rivastigmine, which is chemically designated as Ethylmethyl carbamic acid-3-[(1S)-1-(dimethylamino)ethyl]phenyl ester;

Heloperidol, which is chemically designated as 4-[4-(4-chlorophenyl)-4-hydroxy-1-piperidinyl]-1-(4-fluorophenyl)-1-butanone;

Phenothiazine, which is chemically designated as 10H-Phenothiazine; Thiodiphenyl Amine;

Reserpine, which is chemically designated as 3,4,5-Trimethoxybenzoyl methyl reserpate;

Tetrabenazene, which is chemically designated as 1,3,4,6,7,11b-Hexahydro-9,10-dimethoxy-3-(2-methylpropyl)-2H-benzo[a]quinolizin-2-one;

Bromocryptine, which is chemically designated as 2-bromo-alpha-ergocryptine;

Tiapride, which is chemically designated as N-[2-(Diethylamino)ethyl]-2-methoxy-5-(methylsulfonyl)-o-anisamide;

Baclofen, which is chemically designated as beta-(Aminomethyl)-4-chlorobenzenepropanoic acid;

Diazepam, which is chemically designated as 7-Chloro-1,3-dihydro-1-methyl-5-phenyl-2H-1,4-benzodiazepin-2-one;

Trihexyphenidyl HCl, which is chemically designated as alpha-cyclohexyl-alpha-phenyl-1-piperadinepropanol hydrochloride;

Amitrityline, which is chemically designated as 3-(10,11-Dihydro-5H-dibenzo[a,d]cyclohepten-5-ylidene)-N,N-dimethyl-propanamine;

Amphetamines, which is chemically designated as Alpha-methylbenzeneethanamine;

Methylphenidate, which is chemically designated as alpha-phenyl-2-piperidineacetic acid methyl ester;

Amitriptylinec, which is chemically designated as 3-(10,11-Dihydro-5H-dibenzo[a,d]cyclohepten-5-ylidene)-N,N-dimethyl-1-propanamine;

Clomipramine, which is chemically designated as 3-Chloro-10,11-dihydro-N,N-dimethyl-5H-dibenz[b,f]azepine-5-propanamine;

Dolasetron, which is chemically designated as 1H-indole-3-carboxylic acid (2alpha,6alpha,8alpha,9alphabeta)-octahydro-3-oxo-2,6-methano-2H-quinolizin-8-yl ester;

Granisetron, which is chemically designated as 1-methyl-N-[(3-endo)-9-methyl-9-azabicyclo[3.3.1]non-3-yl]-1H-indazole-3-carboxamide;

Huperzine, an herb used for dementia;

Metoclopramide, which is chemically designated as 4-Amino-5-chloro-N-[(2-diethylamino)ethyl]-2-methoxybenzamide;

Prochlorperazine, which is chemically designated as 2-Chloro-10[3-(4-methyl-1-piperazenyl)propyl]phenothiazene;

Dexamethasone, which is chemically designated as (11beta,16alpha)-9-Fluoro-11,17,21-trihydroxy-16-methylpregna-1,4-diene-3,20-dione;

Timolol Hydrogen maleate salt, which is chemically designated as (2S)-1-[(1,1-Dimethylethyl)amino]-3-[(4-(4-morpholinyl)-1,2,5-thiadiazol-3-yl]oxy]-2-propanol;

Propanolol, which is chemically designated as 1-[(1-Methylethyl)amino]-3-(1-naphthalenyloxy)-2-propanol;

Isometheptine, which is chemically designated as N,6-Dimethyl-5-hepten-2-amine;

Atenolol, which is chemically designated as 4-[2-Hydroxy-3-[(1-methylethyl)amino]propoxy]benzeneacetamide;

Metoprolol, which is chemically designated as 1-[4-(2-Methoxyethyl)-phenoxy]-3-[(1-methylethyl)amino]-2-propanol;

Nadolol, which is chemically designated as 5-[3-[(1,1-Dimethylethyl)-amino]-2-hydroxypropoxy]-1,2,3,4-tetrahydro-2,3-naphthalenediol;

Ergotamine, which is chemically designated as (51alpha)-12′Hydroxy-2′-methyl-(phenylmethyl)argotaman-3′,6′,18-trione;

Dihydroargotamine, which is chemically designated as 9,10-Dihydro-12′-hydroxy-2′-methyl-5′-(phenylmethyl)argotaman-3′,6′,18-trione;

Naratriptan, which is chemically designated as N-Methyl-3-(1-methyl-4-piperidinyl)-1H-indole-5-ethanesulfanamide;

Sumatriptan, which is chemically designated as 3-[2-(Dimethylamino)ethyl]-N-methyl-1H-indole-5-methanesulfonamide;

Rizatriptan, which is chemically designated as N,N-Dimethyl-5-(1H-1,2,4-triazol-1-ylmethyl)-1H-indole-3-ethanamine;

Zolmitriptan, which is chemically designated as (4S)-4-[[3-[2-(Dimethylamino)ethyl]-1H-indol-5-yl)methyl]-2-oxazolidinone;

Imipramine HCl, which is chemically designated as 10,11-Dihydro-N,N-dimethyl-5H-dibenz[b,f]azipine-5-propanamine;

Dopamine, which is chemically designated as 4-(2-Aminoethyl)-1,2 benzenediol;

Clozapine, which is chemically designated as 8-Chloro-11-(4-methyl-1-piperazenyl0-5H-dibenzo[b,f][1,4]diazepine;

Valproic Acid, which is chemically designated as 2-Propylpentanoic Acid;

Amitriptylinec, which is chemically designated as 3-(10,11-Dihydro-5H-dibenzo[a,d]cyclohepten-5-ylidene)-N,N-dimethyl-1-propanamine;

Imipramine HCl which is chemically designated as 10,11-Dihydro-N,N-dimethyl-5H-dibenz[b,f]azipine-5-propanamine;

Imipramine Pamoate, which is chemically designated as 5-(3-dimethylaminopropyl)-10,11-dihydro-5H-dibenz[b,f]azepine

Clomipramine, which is chemically designated as 3-Chloro-10,11-dihydro-N,N-dimethyl-5H-dibenz[b,f]azepine-5-propanamine;

Amphetamine, which is chemically designated as Alpha-methylbenzeneethanamine;

Methylphenidate, which is chemically designated as alpha-phenyl-2-piperidineacetic acid methyl ester;

Phenyloin, which is chemically designated as 5,5-Diphenyl-2,4-imidazolidinedione; Diphenylhydantoin;

Phenobarbital, which is chemically designated as 5-Ethyl-5-phenyl-2,4,6(1H,3H,5H)-pyrimidinetrione;

Amitryptyline, which is chemically designated as 3-(10,22-Dihydro-5H-dibenzo[a,d]cyclohepten-5-ylidene)-N,N-dimethyl-1-propanamine;

Imipramine Pamoate, which is chemically designated as 5-(3-dimethylaminopropyl)-10,11-dihydro-5H-dibenz[b,f]azepine;

Nortrityline, which is chemically designated as 3-(10,11-Dihydro-5H-dibenzo[a,d]cyclohepten-5ylidene-Nmethyl-1-propanamine;

Trazodone, which is chemically designated as 2-[3-[4-(3-Chlorophenyl)-1-piperazinyl]propyl]-1,2,4-triazolo[4,3-a]pyridin-3(2H)-one;

Nefazodone, which is chemically designated as 2-[3-[4-(3-Chlorophenyl)-1-piperazenyl]propyl]-5-ethyl-2,4-dihydro-4-(2-phenoxyethyl)-3H-1,2,4-triazol-3-one;

Sertraline, which is chemically designated as (1S,4S)-4-(3,4-Dichlorophenyl)-1,2,3,4-tetrahydro-n-methyl-1-naphthalenamine;

Fluoxetine, which is chemically designated as 4-[3-[2-(trifluoromethyl)-9H-thioxenthen-9-ylidene]propyl]piperazineethanol;

Paroxetine, which is chemically designated as (3S-trans)-3-[(1,3-Benzodioxol-5-yloxy)methyl]-4-(4-fluorophenyl)piperidine;

Phenalzine, which is chemically designated as (2-Phenethyl)hydrazine;

Tranylcypromine, which is chemically designated as (1R,2S)-rel-2-Phenylcyclopropanamine;

Erythropoietin, which is a Glycoprotein;

Immunoglobulins which are Gama Globulins;

Tetrahydrocannabinols, which is chemically designated as Tetrahydro-6,6,9-trimethyl-3-pentyl-6H-dibenzo[b,d]pyron-1-ol;

Alitretinoin, which is chemically designated as 9-cis-Retinoic Acid; 6-cis-Retinoic Acid;

Lamivudin, which is chemically designated as (2R-cis)-4-Amino-1-[2-(hydroxymethyl)-1,3-oxathiolan-5-yl]-2(1H)-pyrirmidinone;

Stavudin, which is chemically designated as 2′,3′-Didehydro-3′-deoxythymidine;

Zalcitabine, which is chemically designated as 2′3′-Dideoxycytidine; Dideoxycytidine;

Abacavir, which is chemically designated as (1S,4R)-4-[2-Amino-6-(cyclopropylammo)-9H-purin-9-yl]-2-cyclopentene-1-methanol;

Ritonavir, which is chemically designated as (5S,8S,10S,11 S)-10-Hydroxy-2-methyl-5-(1-methylethyl)-1-[2-(1-methylethyl)-4-thiazolyl]-3,6-dioxo-8,11-bis (phenylmethyl)-2,4,7,12-tetraazatridecan-13-oic acid-5-thiazolylmethyl ester;

Indinavir, which is chemically designated as 2,3,5-Trideoxy-N-[(1S,2R)-2,3-dihydro-2-hydroxy-1H-inden-1-yl]-5-[(2S)-2-[{(1,1-dimethylethyl)amino]carbonyl]-4-(3-pyridinylmethyl)-1-piperazenyl]-2-(phenylmethyl)-D-erythro-pentonamide; and

Nelfinavir, which is chemically designated as (3S,4aS,8aS)—N-(1,1-Dimethylethyl)decahydro-2-[(2R,3R)-2-hydroxy-3-[(3-hydroxy-2-methylbenzoy) amino]-4-(phenylthio)butyl]-3-isoquinolinecarboxamide.

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (e.g., sodium, potassium or lithium) or alkaline earth metal (e.g., calcium) salts of carboxylic acids can also be made.

The compounds of formula (I) can be formulated as pharmaceutical compositions and administered to a mammalian host, such as a human patient in a variety of forms adapted to the chosen route of administration, i.e., transnasally, intranasally, orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered transnasally or intranasally. This method administration is particularly well suited for good brain penetration of the active compound.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (e.g., glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying, techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) of formula I in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently administered in unit dosage form; for example, containing 5 to 1000 mg, conveniently 10 to 750 mg, most conveniently, 50 to 500 mg of active ingredient per unit dosage form.

Ideally, the active ingredient should be administered to achieve peak plasma concentrations of the active compound of from about 0.5 to about 75 μM, preferably, about 1 to 50 μM, most preferably, about 2 to about 30 μM, This may be achieved, for example, by the intravenous injection of a 0.05 to 5% solution of the active ingredient, optionally in saline, or orally administered as a bolus containing about 1-100 mg of the active ingredient, Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The ability of a compound of the invention to act as an MMP inhibitor may be determined using pharmacological models which are well known to the art, or using the methods described herein below.

### Fluorescence Enzymatic Activity Assays

The enzymatic activity of MMP-2, MMP-9, and MMP-7 was monitored with the fluorescence quenched substrate MOCAcPLGLA₂pr(Dnp)-AR-NH₂. Fluorescence was measured with a Photon Technology International (PTI) spectrofluorometer interfaced to a Pentium computer, equipped with the RatioMaster™ and FeliX™ hardware and software, respectively. The cuvette compartment was thermostated at 25.0° C. Substrate hydrolysis was monitored at emission and excitation wavelengths of 328 and 393 nm and excitation and emission band passes of 1 and 3 nm, respectively. Fluorescence measurements were taken every 4 s. Less than 10% hydrolysis of the fluorogenic substrate was monitored, as described by Knight. Knight, C. G. Methods Enzymol 1995,248, 18-34. Stromelysin 1 enzymatic activity was monitored using the synthetic fluorogenic substrate MOCAcRPKPVE-Nva-WRK(Dnp)-NH₂ (Peptides International, Louisville, Ky.; RPKPVENRWRK is represented by SEQ ID NO:4) at excitation and emission wavelengths of 325 and 393 nm and excitation and emission band passes of 1 and 3 nm, respectively.

### Enzymes and Protein Inhibitors.

Human pro-MMP-2, pro-MMP-9, TIMP-1 and TIMP-2 were expressed in HeLa S3 cells infected with the appropriate recombinant vaccinia viruses and were purified to homogeneity, as previously described. Fridman, R.; Fuerst, T. R.; Bird, R. E.; Hoyhtya, M.; Oelkuct, M.; Kraus, S.; Komarek, D.; Liotta, L. A.; Berman, M. L.; Stetler-Stevenson, W. G. J. Biol. Chem. 1992, 267, 15398-15405. Fridman, R.; Birs, R. E.; Hoyhtya, M.; Oelkuct, M.; Komarek, D.; Liang, C. M.; Berman, M. L.; Liotta, L. A.; Stetler-Stevenson, W. G.; Fuerst, T. R. Biochem. J. 1993, 289, 411-416. Pro-MMP-2, pro-MMP-9, TIMP-1 and TIMP-2 concentrations were determined using the extinction coefficients of 122,800, 114,360, 26,500 and 39,600M⁻¹cm⁻¹, respectively. To obtain active MMP-2, pro-MMP-2 (7.3 μM) was incubated at 37° C. for 1 h with 1 mM p-aminophenylmercuric acetate (APMA) (dissolved in 200 mM Tris) in buffer C. The enzyme solution was dialyzed against buffer D at 4° C. to remove APMA. Active MMP-9 was obtained by incubating pro-MMP-9 (1 μM) with heat-activated recombinant human stromelysin 1 (68 nM) (MMP-3, generously provided by Dr. Paul Cannon, Center for Bone and Joint Research, Palo Alto, Calif.) at 37° C., for 2.5 h in buffer C.

The resulting solution was subjected to gelatin-agarose chromatography to remove stromelysin 1. MMP-9 was eluted with buffer D containing 10% DMSO and dialyzed against the same buffer without DMSO to remove the organic solvent. Pro-MMP-2 and pro-MMP-9 activation reactions were monitored using the fluorescence quenched substrate MOCAcPLGLA₂pr(Dnp)-AR—NH₂ (Peptides International, Louisville, Ky.; PLGLAAAR is represented by SEQ ID NO:5), as will be described below. The MMP-2 and MMP-9 concentrations were determined by titration with TIMP-1.

### Kinetic Analyses.

Progress curves were obtained by adding enzyme (0.5-2 nM) to a mixture of fluorogenic substrate (5-7 μM) and varying concentrations of inhibitor in buffer R containing 5-15% DMSO (final volume 2 ml), in acrylic cuvettes with stirring and monitoring the increase in fluorescence with time for 15-30 minutes. The progress curves were nonlinear least squares fitted to Equation 1 (Muller-Steffner, H. M., Malver, O., Hosie, L., Oppenheimer, N. J., and Schuber, F. J. Biol. Chem. 1992, 267, 9606-9611.):

F=νₛt+I(νₒ−νₛ)(I−exp(−kt))/k+F₀ (1)

where ν₀ represents the initial rate, νₛ, the steady state rate, k, the apparent first order rate constant characterizing the formation of the steady-state enzyme-inhibitor complex and Fₒ, the initial fluorescence, using the program SCIENTIST (MicroMath Scientific Software, Salt Lake City, Utah). The obtained k values, ν₀ and νₛ were further analyzed according to Equations 2 and 3 for a one-step association mechanism

k=k_{off}+kₒₙ[I]/(1+[S]/Kₘ) (2)

(νₒ−νₛ)/νₛ=[I]/(Kᵢ(1+[S]/Kₘ)) (3)

Intercept and slope values, obtained by linear regression of the k versus inhibitor concentration plot (Equation 2), yielded the association and dissociation rate constants kₒₙ and k_{off}, respectively, and the inhibition constant Kᵢ(k_{off}/kₒₙ). Alternatively, Kᵢ was determined from the slope of the (λₒ−νₛ)/νₛ νs [I] plot according to Equation 3.

The dissociation rate constants were determined independently from the enzyme activity recovered after dilution of a pre-formed enzyme-inhibitor complex. To this end, typically 200 nM of enzyme was incubated with 1 μM of inhibitor for a sufficient time to reach equilibrium (>45 min) at 25.0° C. The complex was diluted into 2 mL of buffer R containing fluorogenic substrate (5-7 μM final concentration) to a final enzyme concentration of 1 nM. Recovery of enzyme activity was monitored for ˜30 min. The fluorescence versus time trace was fitted, using the program SCIENTIST, to Equation 4

F=vₛt+(νₒ−νₛ)(1−exp(−k_{off}))/k_{off}+F₀ (4)

where νₒ represents the initial rate (very small), νₛ, the rate observed when the E.I complex is completely dissociated and k_{off}, the first order rate constant when the E.I dissociation.

Analysis for linear competitive inhibition was performed in the following manner. Initial rates were obtained by adding enzyme (0.5-2 nM) to a mixture of fluorogenic substrate (5-7 μM) and varying concentrations of inhibitor in buffer R, containing 5-15% DMSO (final volume 1 mL) in semi-micro quartz cuvettes, and monitoring the increase in fluorescence with time for 5-10 minutes. The fluorescence versus time traces were fitted by linear regression analysis using FeliX™. The initial rates were fitted to Equation 5 (Segel, I. H. in: Enzyme Kinetics, Wiley Inc., New York, 1975, pp. 104.):

v/Vₘₐₓ=S/(Kₘ(1+I/Kᵢ)+S) (5)

where ν and Vₘₐₓ represent the initial and maximal velocities, S and I, the substrate and inhibitor concentrations, respectively, Kₘ the Michaelis-Menten constant for the substrate-enzyme reaction and Kᵢ the inhibition constant, using the program SCIENTIST.

Inhibitors 1-3 all bind with the active site of the MMPs that were used in the study, with Kᵢ values of micromolar, or less, however, the behavior of inhibitor 1 was very different. Inhibitor 1 showed a dual behavior. It served as a mechanism-based inhibitor with a partition ratio of 79±10 (i.e. k_{cat}/k_{inact}) for MMP-2 and 416±63 for MMP-9. Furthermore, it also behaved as a slow-binding inhibitor, for which the rate constants for the on-set of inhibition (kₒₙ) and recovery of activity from inhibition (k_{off}) were evaluated (Table 1). It would appear that coordination of the thiirane with the zinc ion (as seen in energy-minimized computational models; FIG. 1) would set in motion a conformational change, which is presumed from the slow-binding kinetic behavior. The kinetic data fit the model for slow-binding inhibition. Morrison, J. F. Adv. Enzymol 1988, 61, 201-301. Covalent modification of the enzymes results from this conformational change. Inhibitor 1 was incubated with MMP-2 to the point that less than 5% activity remained. This inhibitor-enzyme complex was dialyzed over three days, which resulted in recovery of approximately 50% of the activity. This observation is consistent with modification of the active site Glu-404 (according to the numbering for human MMP-2), via the formation of an ester bond, which is a relatively labile covalent linkage. The time-dependent loss of activity is not merely due to the slow-binding behavior. For instance, for a k_{off} of 2×10⁻³ s⁻¹ (the values are not very different from one another in Table 1) the half time for recovery of activity (t_{1/2}) is calculated at just under 6 min. The fact that 50% of activity still did not recover after dialysis over three days strongly argues for the covalency of enzyme modification.

Selectivity in inhibition of gelatinases by inhibitor 1 was observed. Its Kᵢ values are 13.9±4 nM and 600±200 nM for MMP-2 and MMP-9, respectively. The corresponding Kᵢ values are elevated to the micromolar range for the other MMPs, even for the case of MMP-3, which does show the slow-binding, mechanism-based inhibition profile. In addition, the values for kₒₙ are 611- and 78-fold larger for MMP-2 and MMP-9, respectively, than that for MMP-3. Whereas the k_{off} values are more similar to one another, the value for MMP-2 is the smallest, so the reversal of inhibition of this enzyme takes place more slowly. Collectively, these kinetic parameters demonstrate that inhibitor 1 can be a potent and selective inhibitor for MMP-2, MMP-9, and especially MMP-2. It has been previously shown that two molecules of either TIMP-1 or TIMP-2 (endogenous cellular protein inhibitors of MMPs) bind to activated MMP-2 and MMP-9. Olson, M. W.; Gervasi, D. C.; Mobashery, S.; Fridman, R. J. Biol. Chem. 1997, 272, 29975. One binding event is high affinity and would appear physiologically relevant, whereas the second binding event takes place with relatively lower affinity (micromolar). Olson, M. W.; Gervasi, D. C.; Mobashery, S.; Fridman, R. J. Biol. Chem. 1997, 272, 29975. Inhibition of MMP-2 and MMP-9 by TIMPs also follows slow-binding kinetics. The kinetic parameters for these interactions at the high affinity site are listed in Table 1. The kinetic parameters for the slow-binding component of inhibition of MMP-2 and MMP-9 by inhibitor 1 (Kₒₙ and K_{off}) approach closely the same parameters for those of the protein inhibitors. Olson, M. W.; Gervasi, D. C.; Mobashery, S.; Fridman, R. J. Biol. Chem. 1997, 272, 29975-29983.

Oxiranes 4-6 inhibit MMPs in a competitive manner with higher Kᵢ values. There was no evidence of slow-binding behavior or time-dependence of loss of activity with this inhibitor with any of the MMPs.

Small-molecule inhibitor 1 follows both slow-binding and mechanism-based inhibition in its kinetic profile. This compound appears to behave very similarly to the endogenous cellular protein inhibitors for MMPs (TIMPs) in the slow-binding component of inhibition. Furthermore, the inhibitor also exhibits a covalent mechanism-based behavior in inhibition of these enzymes. The high discrimination in targeting that inhibitor 1 displays (both in affinities and the modes of inhibition) among the other structurally similar MMPs is noteworthy and could serve as a paradigm in the design of inhibitors for other closely related enzymes in the future.

### EXAMPLES

The following examples are provided in order to demonstrate and further illustrate certain embodiments and aspects of the present invention and are not to be construed as limiting the scope thereof.

### Experimental Procedures

¹H and ¹³C NMR spectra were recorded on either a Varian Gemini-300, a Varian Mercury-400 or a Varian Unity-500 spectrometer. Chemical shifts are reported in ppm from tetramethylsilane on the δ scale. Infrared spectra were recorded on a Nicolet 680 DSP spectrophotometer. Mass spectra were recorded on a Kratos MS 80RFT spectrometer. Melting points were taken on an Electrothermal melting point apparatus and are uncorrected. Thin-layer chromatography was performed with Whatman reagents 0.25 mm silica gel 60-F plates. All other reagents were purchased from either Aldrich Chemical Company or Across Organics.

The following buffers were used in experiments with enzymes: Buffer C (50 mM HEPES at pH 7.5, 150 mM NaCl, 5 mM CaCl₂, 0.02% Brij-35); buffer R (50 mM HEPES at pH 7.5, 150 mM NaCl, 5 mM CaCl₂, 0.01% Brij-35, and 1% v/v Me₂SO) and buffer D (50 mM Tris at pH 7.5, 150 mM NaCl, 5 mM CaCl₂, and 0.02% Brij-35).

### Example 1

(4-Phenoxyphenylsulfonyl)methyloxirane (4). To compound 11 (598 mg, 2.5 mmol) in dichloromethane (10 mL), mCPBA (2.84 g, 10 mmol, Aldrich 57-86%), was slowly added. The mixture was stirred at room temperature for 3 days, after which time a second portion of mCPBA (2.84 g, 10 mmol) was added. The mixture was then stirred for another 4 days, after which time the mixture was poured into ethyl acetate (200 mL), and washed with aqueous sodium thiosulfate (3×50 mL, 10% w/v), aqueous sodium bicarbonate (3×50 ml, 5% w/v), and brine (50 ml). The organic phase was dried over magnesium sulfate and was concentrated to provide a yellow oil. The crude material was purified by column chromatography (silica, 4:1 hexanes:ethyl acetate) to give compound 4 as a pale yellow semi-solid (501 mg, 70%). ¹H NMR (500 MHz, CDCl₃) δ 7.90-7.86 (m, 2H), 7.46-7.40 (m, 2H), 7.26-7.22 (m, 1H), 7.10-6.96 (m, 4H), 3.34-3.24 (m, 2H), 2.84-2.80 (m, 1H), 2.49-2.46 (m, 1H); ¹³C NMR (125 MHz, CDCl₃) δ 163.15, 154.95, 130.76, 130.51, 125.52, 120.77, 117.83, 59.89, 46.13; IR(film) 3054 (w), 2919 (w), 1576 (s), 1492 (s), 1320 (s), 1245 (s), 1148 (s) cm⁻¹; m/z (EI) 290 (M⁺, 100%), 233 (70), 217 (50), 185 (40); HRMS (EI) calcd. for C₁₅H₁₄O₄S 290.0613, found 290.0611.

The intermediate, compound 11, was prepared as follows:

(A.) O-4-Phenoxyphenyl-N,N-dimethylthiocarbamate (8). To a solution of 4-phenoxyphenol (7, 8.46 g, 45 mmol) in DMF (40 mL) at 10° C., sodium hydride (1.83 g, 45 mmol, 60% dispersion in mineral oil) was added in small portions. After the evolution of hydrogen ceased, N,N-dimemylthiocarbamoyl chloride (6.16 g, 50 mmol) was added in one portion. The reaction mixture was then stirred at 70° C. for 2 hours. The mixture was cooled to room temperature, poured into water (100 mL) and extracted with chloroform (3×50 mL). The combined organic extracts were washed with aqueous potassium hydroxide (50 mL, 5% w/v), and brine (10×50 mL). The organic extract was dried over magnesium sulfate and concentrated to obtain a yellow oil. The crude material was purified by column chromatography (silica, 5:1 hexanes:ethyl acetate) to give compound 8 as a white solid (11.16 g, 90%). m.p. 50-51° C.; ¹H NMR (300 MHz, CDCl₃) δ 7.38-7.31 (m, 2H), 7.14-7.08 (m, 1H), 7.06-7.00 (m, 6H), 3.46 (s, 3H), 3.34 (s, 3H); ¹³C NMR (75 MHz, CDCl₃) δ 188.17, 157.26, 155.16, 149.62, 130.05, 124.11, 123.71, 119.31, 43.57, 38.96; IR (KBr) 3040 (m), 2938 (s), 1587 (s), 1487 (s), 1394 (s), 1287 (s), 1190 (s) cm⁻¹; m/z (EI) 273 (M⁺, 15%), 186 (100); HRMS (EI) calcd. for C₁₅H₁₅NO₂S 273.0823, found 273.0824.

(B.) S-4-Phenoxyphenyl-N,N-dimethylthiocarbamate (9). Compound 8 (3.99 g, 15 mmol) was heated under argon at 260° C. for 3.5 hours. The resulting dark brown oil was purified by column chromatography using a gradient eluent system (silica, 19:1 then 9:1 then 3:1 hexanes:ethyl acetate) to obtain compound 9 as a pale yellow solid (2.55 g, 64%). m.p. 97-99° C.; ¹H NMR (400 MHz, CDCl₃) δ 7.45-7.40 (m, 2H), 7.40-7.30 (m, 2H), 7.15-7.10 (m, 1H), 7.05 (d, J=8.8 Hz, 2H) 6.98 (d, J=8.8 Hz, 2H) 3.08 (bs, 3H), 3.02 (bs, 3H); ¹³C NMR (100 MHz, CDCl₃) δ 167.48, 158.87, 156.53, 137.66, 130.09, 124.14, 122.39, 119.87, 118.94, 37.14; IR(KBr) 3037 (w), 2925 (w), 1652 (s), 1581 (s) 1486 (s), 1239 (s) cm⁻¹; m/z (EI) 273 (M⁺, 25%), 257 (5), 200 (5); HRMS (EI) calcd. for C₁₅H₁₅NO₂S 273.0823, found 273.0822.

(C.) 4-Phenoxythiophenol (10). A mixture of compound 9 (2.55 g, 9 mmol) in methanol (20 mL), and aqueous NaOH (10 mL, 10% w/v), were refluxed for 4 hours. The solution was cooled to room temperature and was acidified to pH 1 with aqueous HCl (1M). Water (100 mL) was added and the mixture was extracted with chloroform (3×50 mL). The combined organic extracts were washed with brine (50 mL), dried over magnesium sulfate and concentrated to obtain a yellow oil. The crude product was purified by column chromatography (silica, 5:1 hexanes:ethyl acetate) to give compound 10 as a pale yellow oil (1.80 g, >99%). ¹H NMR (300 MHz, CDCl₃) δ 7.36-7.31 (m, 2H), 7.30-7.25 (m, 2H), 7.13-7.09 (m, 1H), 7.04-6.88 (m, 4H), 3.43 (s, 1H); ¹³C NMR (75 MHz, CDCl₃) δ 157.30, 156.15, 132.14, 130.00, 124.04, 123.95, 119.88, 119.04; IR(film) 3038 (w), 1583 (s), 1484 (s), 1236 (s), 1166 (s) cm⁻¹; m/z (EI) 202 (M⁺, 100%; HRMS (EI) calcd. for C₁₂H₁₀OS 202.0452, found 202.0454.

(D.) 3-(4-Phenoxyphenylsulfanyl)-1-propene (11). To a mixture of compound 10 (516 mg, 2.7 mmol) and potassium carbonate (534 mg, 3.9 mmol) in DMF (5 mL), allyl bromide (253 μL, 2.9 mmol) was added in one portion. The mixture was stirred at room temperature overnight. The crude reaction mixture was poured into ether (200 mL), washed with saturated aqueous potassium carbonate (25 mL), and brine (6×50 mL). The organic layer was dried over magnesium sulfate and concentrated in vacuo to give a yellow oil. The crude material was purified by column chromatography (silica, 98:2 hexanes:ethyl acetate) to obtain the title compound as a pale yellow oil (598 mg, 93%). ¹H NMR (300 MHz, CDCl₃) δ 7.38-7.32 (m, 4H), 7.15-7.10 (m, 1H), 7.04-7.00 (m, 2H), 6.97-6.92 (m, 2H), 5.92-5.82 (m, 1H), 5.10-5.04 (m, 2H), 3.50 (d, J=7.2 Hz, 2H); ¹³C NMR (75 MHz, CDCl₃) δ 157.14, 156.73, 134.01, 133.22, 130.05, 129.50, 123.75, 119.40, 119.25, 117.81, 38.84; IR(film) 3078 (w), 3039 (w), 1582 (s), 1484 (s), 1240 (s), 1165 (s) cm⁻¹; m/z (EI) 242 (M⁺, 100%), 201 ([M-allyl]⁺, 100); HRMS (EI) calcd. for C₁₅H₁₄OS 242.0765, found 242.0764.

### Example 2

2-(4-Phenoxyphenylsulfonyl)ethyloxirane (5). The title compound was prepared in the same manner as described for 4, with the exception that compound 12 was used in place of compound 11, and the reaction time was 2 days. The title compound was obtained as a white solid (78%). m.p. 75-77° C.; ¹H NMR (500 MHz, CDCl₃) δ 7.84-7.80 (m, 2H), 7.44-7.38 (m, 2H), 7.24-7.20 (m, 1H), 7.09-7.04 (m, 4H), 3.25-3.15 (m, 2H), 3.02-2.97 (m, 1H), 2.76 (t, J=4.3 Hz, 1H), 2.49 (dd, J= 3.0 and 5.0 Hz, 1H), 2.19-2.10 (m, 1H), 1.86 (m, 1.H); ¹³C NMR (125 MHz, CDCl₃) δ 162.93, 155.02, 130.58, 130.81, 125.47, 120.69, 117.91, 53.15, 50.32, 47.29, 26.23; IR(KBr disc) 3040 (s), 1580 (s), 1490 (s), 1320 (s), 1248 (s), 1148 cm⁻¹; m/z (EI) 304 (M⁺, 80%), 233 (50), 217 (100); HRMS (EI) calcd. for C₁₆H₁₆O₄S 304.0769, found 304.0768.

(A.) 4-(4-Phenoxyphenylsulfanyl)-1-butene (12). The title compound was prepared in the same manner as described for 11, with the exception that 4-bromo-1-butene was used in place of allyl bromide. Compound 12 was obtained as a colorless oil (88%). ¹H NMR (400 MHz, CDCl₃) δ 7.37-7.32 (m, 4H), 7.14-7.10 (m, 1H), 7.04-7.00 (m, 2H), 6.96-6.88 (m, 2H), 5.90-5.80 (m, 1H), 5.12-5.02 (m, 2H), 2.98 (m, 2H), 2.41-2.34 (m, 2H); ¹³C NMR (100 MHz, CDCl₃) δ 157.18, 156.50, 136.65, 132.57, 130.05, 123.72, 119.55, 119.21, 116.47, 34.65, 33.71; IR(film) 3076 (w), 2923 (w), 1583 (s), 1485 (s), 1239 (s) cm⁻¹; m/z (EI) 256 (M⁺, 100%), 215 ([M-allyl]⁺, 90), 202 (15); HRMS (EI) calcd. for C₁₆H₁₆OS 256.0922, found 256.0922.

### Example 3

3-(4-Phenoxyphenylsulfonyl)propyloxirane (6). The title compound was prepared in the same manner as described for 4, with the exception that compound 13 was used in place of compound 11, and that the reaction time was 3 days. The title compound was obtained as a white solid (94%). ¹H NMR (500 MHz, CDCl₃) δ 7.86-7.80 (m, 2H), 7.44-7.39 (m, 2H), 7.25-7.22 (m, 1H), 7.10-7.04 (m, 4H), 3.21-3.08 (m, 2H), 2.90-2.86 (m, 1H), 2.74 (t, J= 4.5 Hz, 1H), 2.45 (dd, J= 2.5 and 4.5 Hz, 1H), 1.92 (quin, J= 7.0 Hz, 2H), 1.85-1.78 (m, 1H), (m, 1H); ¹³C NMR (125 MHz, CDCl₃) δ 162.84, 155.08, 130.58, 130.48, 125.43, 120.70, 117.88, 56.28, 51.64, 46.86, 31.17, 20.12; IR(KBr disc) 3063 (w), 2923 (w), 1582 (s), 1488 (s), 1294 (s), 1246 (s), 1142 (s) cm⁻¹; m/z (EI) 318 (M⁺, 40%), 290 (20), 217 (100%); HRMS (EI) calcd. for C₁₇H₁₈O₄S 318.0926, found 318.0924.

(A.) 5-(4-Phenoxyphenylsulfanyl)-1-pentene (13). The title compound was prepared in the same manner as described for 11, with the exception that 5-bromo-1-pentene was used in place of allyl bromide. The title compound was obtained as a colorless oil (65%). ¹H NMR (500 MHz, CDCl₃) δ 7.37-7.34 (m, 4H), 7.13-7.09 (m, 1H), 7.03-7.00 (m, 2H), 6.96-93 (m, 2H), 5.83-5.74 (m, 1H), 5.06-4.98 (m, 2H), 2.88 (t, J=7.0 Hz, 2H), 2.22-2.16 (m, 2H), 1.73 (q, J=7.0 Hz, 2H); ¹³C NMR (125 MHz, CDCl₃) δ 157.23, 156.36, 137.84, 132.30, 130.41, 130.03, 123.67, 119.55, 119.16, 115.62, 34.61, 32.86, 28.61; IR(film) 3075 (w), 2929 (m),1583 (s), 1484 (s), 1236 (s) cm⁻¹; m/z (EI) 270 (M⁺, 100%), 215 (70), 202 (60); HRMS (EI) calcd. for C₁₇H₁₈OS 270.1078, found 270.1076.

### Example 4

(4-Phenoxyphenylsulfonyl)methylthiirane (1). To a solution of compound 4 (710 mg, 2.5 mmol) in THF (5 mL), a solution of ammonium thiocyanate (559 mg, 7.4 mmol) in water (3 mL) was added. The reaction was stirred at room temperature for 16 hours, after which time it was poured into ethyl acetate (100 mL), and then washed with water (25 mL), followed by brine (25 mL). The organic phase was dried over magnesium sulfate and was concentrated to give a white oil. The crude material was purified by column chromatography (silica, 8:1 hexanes:ethyl acetate) to obtain compound 1 as a white solid (102 mg, 14%). m.p. 99-101° C.; ¹H NMR (500 MHz, CDCl₃) δ7.89-7.84 (m, 2H), 7.46-7.40 (m, 2H), 7.26-7.22 (m, 1H), 7.11-6.96 (m, 4H), 3.52 (dd, J=5.5 and 14.5 Hz, 1H), 3.17 (dd, J=7.5 and 14.5 Hz, 1H), 3.09-3.03 (m, 1H), 2.53 (dd, J=2.0 and 6.0 Hz, 1H) 2.16 (dd, J= 2.0 and 5.0 Hz, 1H); ¹³C NMR (125 MHz, CDCl₃) δ 163.20, 155.02, 132.13, 130.95, 130.52, 125.52, 120.69, 117.97, 62.90, 26.31, 24.47; IR(KBr disc) 3030 (w), 1583 (s), 1486 (s), 1317 (s), 1246 (s), 1141 (s) cm⁻¹; m/z (EI) 306 (M⁺, 2%), 242 ([M-SO₂]⁺, 35); HRMS (EI) calcd. for C₁₅H₁₄O₃S₂ 306.0384, found 306.0382.

### Example 5

2-(4-Phenoxyphenylsulfonyl)ethylthiirane (2). The title compound was prepared in the same manner as described for 1, with the exception that compound 5 was used in place of compound 4. The crude material was purified by column chromatography (silica, 2:1 hexanes:ethyl acetate) to give the title compound as a white solid (93%). m.p. 99-101° C.; ¹H NMR (500 MHz, CDCl₃) δ 7.83 (d, J= 8.0 Hz, 2H), 7.42 (t, J=8.0 Hz, 2H), 7.26-7.22 (m, 1H), 7.10-7.06 (m, 4H), 3.30-3-20 (m, 2H), 2.98-2.92 (m, 1H), 2.52 (dd, J=1 and 6 Hz, 1H), 2.48-2.39 (m, 1H), 2.18 (dd, J=1 and 5 Hz, 1H), 1.78-1.69 (m, 1H); ¹³C NMR (125 MHz, CDCl₃) δ 162.94, 155.03, 132.50, 130.55, 130.51, 125.48, 120.71, 117.92, 55.97, 33.62, 29.82, 26.05; IR(KBr disc) 3040 (w), 1583 (s), 1487 (s), 1256 (s), 1142 (s) cm⁻¹; m/z (EI) 320 (M⁺, 50%), 288 (20), 234 (40), 217 (60), 170 (100); HRMS (EI) calcd. for C₁₆H₁₆O₃S₂ 320.0541, found 320.0540.

### Example 6

3-(4-Phenoxyphenylsulfonyl)propylthiirane (3). The title compound was prepared in the same manner as described for 1, with the exception that compound 6 was used in place of compound 4. The crude material was purified by column chromatography (silica, 2:1 hexanes:ethyl acetate) to give the title compound as a white solid (85%). m.p. 75-76° C.; ¹H NMR (500 MHz, CDCl₃) δ 7.85-7.82 (m, 2H), 7.44-7.40 (m, 2H), 7.26-7.22 (m, 1H), 7.10-7.06 (m, 4H), 3.20-3.09 (m, 2H), 2.84-2.79 (m, 1H), 2.50 (dd, J=1 and 6 Hz, 1H), 2.14 (dd, J=1 and 5.5 Hz, 1H), 2.12-2.06 (m, 1H), 1.97 (quin, J=8 Hz, 2H), 1.45-1.38 (m, 1H); ¹³C NMR (125 MHz, CDCl₃) δ 162.85, 155.08, 132.55, 130.60, 130.49, 125.43, 120.69, 117.91, 56.09, 35.13, 34.86, 25.72, 22.92; IR(KBr disc) 3000 (w), 1583 (s), 1480 (s), 1254 (s), 1143 (s) cm⁻¹; m/z (EI) 334 (M⁺, 30%), 301 (10), 234 (100), 217 (70), 170 (70); HRMS (EI) calcd. for C₁₇H₁₈O₃S₂ 334.0697, found 334.06.

### Example 7

TABLE 1
Kinetics parameters for inhibition of MMPs
by compounds of the present invention

| | | |
|---|---|---|
| kₒₙ (M⁻¹s⁻¹) × 10⁻⁴ | k_{off}(s⁻¹) × 10³ | Kᵢ (μM) |

Compound 1

| | | | |
|---|---|---|---|
| MMP-2 | 11 ± 1 | 1.5 ± 0.6^{a} | 0.0139 ± 0.0004 |
| | | 1.8 ± 0.1 | |
| MMP-9 | 1.4 ± 0.3 | 9 ± 1^{a} | 0.6 ± 0.2 |
| | | 7.1 ± 0.5 | |
| MMP-3 | (1.8 ± 0.4) × 10⁻² | 2.7 ± 0.9^{a} | 15 ± 6 |
| | | 5.5 ± 0.4 | |
| MMP-7 | | | 96 ± 41 |

Compound 2

| | |
|---|---|
| MMP-2 | 4.7 ± 0.7 |
| MMP-9 | 44 ± 5 |
| MMP-3 | NI^{b} |
| MMP-7 | NI |
| MMP-1 | NI |

Compound 3

| | |
|---|---|
| MMP-2 | 4.3 ± 0.7 |
| MMP-9 | 181 ± 41 |
| MMP-3 | NI |
| MMP-7 | NI |
| MMP-1 | NI |

Compound 4

| | |
|---|---|
| MMP-2 | 25 ± 2 |
| MMP-9 | 186 ± 11 |
| MMP-3 | NI |
| MMP-7 | NI |
| MMP-1 | NI |

TIMP-1^{c}

| | | | |
|---|---|---|---|
| MMP-2 | 4.4 ± 0.1 | 1.3 ± 0.2 | 0.029 ± 0.005 |
| MMP-9 | 5.2 ± 0.1 | 1.2 ± 0.2 | 0.024 ± 0.004 |

TIMP-2^{c}

| | | | |
|---|---|---|---|
| MMP-2 | 3.3 ± 0.1 | 0.8 ± 0.1 | 0.023 ± 0.004 |
| MMP-9 | 2.2 ± 0.1 | 1.3 ± 0.2 | 0.058 ± 0.007 |

Compound 5

| | |
|---|---|
| MMP-2 | 5.1 ± 0.5 |
| MMP-9 | 102 ± 2 |
| MMP-3 | NI^{a} |
| MMP-7 | NI |
| MMP-1 | NI |

Compound 6

| | |
|---|---|
| MMP-2 | 10.7 ± 0.6 |
| MMP-9 | 75 ± 6 |
| MMP-3 | NI^{b} |
| MMP-7 | NI |
| MMP-1 | NI |

^{a}Determined by two different methods, hence the set of two numbers.
^{b}NI, for “not inhibiting”, even at high concentrations of 130-330 μM.
^{c}Kinetic parameters for the high-affinity site for TIMPs were reported earlier in Olson, M. W.; Gervasi, D. C.; Mobashery, S.; Fridman, R., J. Biol. Chem., 1997, 272, 29975-29983.

### Example 8

### Matrix Metalloproteinase S-Nitrosylation & Neuron Death

Introduction

Evidence is accumulating that damage and apoptotic death of neurons plays a role in the pathogenesis of many acute and chronic neurologic disorders. These disorders range from acute stroke, head trauma and epilepsy to more chronic states, such as Huntington's disease, Alzheimer's disease, HIV-associated dementia, multiple sclerosis, and glaucoma. Moreover, a contributing factor to several of these diseases is the activation of matrix metalloproteinases (MMPs) in the extracellular matrix.

MMPs constitute a family of extracellular soluble or membrane-bound proteases that are prominently involved in remodeling extracellular matrix (ECM). MMP-9 in particular is significantly elevated in humans after stroke (Montaner, J.; Alvarez-Sabin, J.; Molina, C; Angles, A.; Abilleira, S.; Arenillas, J.; Gonzalez, M. A.; Monasterio, J. Stroke 2001, 32, 1759-1766). Mice deficient in MMP-9 manifest a reduction in cerebral infarct size; in addition, treatment with broad-spectrum MMP inhibitors or antibodies also reduces infarct size (Romanic, A. M.; White, R. F.; Arleth, A. J.; Ohlstein, E. H.; Barone, F. C. Stroke 1998, 29, 1020-1030; Asahi, M.; Asahi, K.; Jung, J. C; del Zoppo, G. J.; Fini, M. E.; Lo, E. H. Journal of Cerebral Blood Flow & Metabolism 2000, 20, 1681-1689; Gasche, Y.; Fujimura, M.; Morita-Fujimura, Y.; Copin, J. C; Kawase, M.; Massengale, J.; Chan, P. H. Journal of Cerebral Blood Flow & Metabolism 1999, 19, 1020-1028). Additionally, MMP-2 levels are acutely increased in the brains of baboons after stroke (Heo, J. H.; Lucero, J.; Abumiya, T.; Koziol, J. A.; Copeland, B, R.; del Zoppo, G. J. Journal of Cerebral Blood Flow & Metabolism 1999, 19, 624-633). Members of the MMP family share several structural features including propeptide, catalytic, and hemopexin domains (except MMP-7 which lacks the latter domain). In each case, one cysteine residue in the conserved autoinhibitory region of the propeptide domain coordinates a catalytic zinc ion lying at the catalytic center of the enzyme. This cysteine replaces a Zn²⁺-bound water molecule that is the nucleophile in peptide bond hydrolysis by MMPs, thus inhibiting activity of the proform of the enzyme, Disruption of the Zn²⁺-cysteine interaction exposes Zn²⁺ in the active site allowing H₂O to bind, and consequently activates the MMP zymogen by a mechanism known as the “cysteine switch” (Morgunova, E.; Tuuttila, A.; Bergmann, U.; Isupov, M.; Lindqvist, Y.; Schneider, G.; Tryggvason, K. Science 1999, 284, 1667-1670; Van Wart, H. E.; Birkedal-Hansen, H. Proceedings of the National Academy of Sciences of the United States of America 1990, 87, 5578-5582). Under physiological conditions, MMP activity is also controlled by tissue inhibitors of MMPs (TIMPs) (Yong, V. W.; Krekoski, C. A.; Forsyth, P. A.; Bell, R.; Edwards, D. R. Trends in Neurosciences 1998, 21, 75-80; Lukes, A.; Mun-Bryce, S.; Lukes, M.; Rosenberg, G. A. Molecular Neurobiology 1999, 19, 267-2S4). Imbalance of MMP activity levels is thought to underlie many neurodegenerative disorders as well as other inflammatory and malignant diseases (Yong, V, W.; Krekoski, C. A.; Forsyth, P. A.; Bell, R.; Edwards, D. R. Trends in Neurosciences 1998, 21, 75-80; Lukes, A.; Mun-Bryce, S.; Lukes, M.; Rosenberg, G. A. Molecular Neurobiology 1999, 19, 267-284). However, the pathophysiological mechanism of MMP activation in diseases has remained an enigma and the rote of MMP activation in neuronal damage has been unknown.

Nitric oxide (NO) is a signaling molecule implicated in regulation of many biological processes in the nervous system, including neurotransmitter release, plasticity, and apoptosis (Dawson, T. M.; Snyder, S. H. Journal of Neuroscience 1994, 14, 5147-5159; Lipton, S. A.; Choi, Y. B.; Pan, Z. H; Lei, S. Z.; Chen, H. S.; Sucher, N. J.; Loscalzo, J,; Singel, D. J.; Stamler, J. S. Nature 1993, 364, 626-632; Melino, G. Bernassola, F.; Knight, R. A.; Corasaniti, M. T.; Nistico, G,; Finazzi-Agro, A. Nature 1997, 388, 432-433). The chemical reactions of NO are largely dictated by its redox state (Stamler, J. S. Cell 1994, 78, 931-936). NO has been shown to modulate the biological activity of many proteins by reacting with cysteine thiol to form an S-nitrosylated derivative. Such reactions regulate the activity of circulating, membrane-bound, cytosolic, and nuclear proteins, including hemoglobin, NMDA receptors, caspases, and NF-B (Jia, L.; Bonaventura, C; Bonaventura, J.; Stamler, J. S. Nature 1996, 380, 221-226; Choi, Y. B.; Tenneti, L.; Le, D. A.; Ortiz, J.; Bai, G.; Chen, H. S.; Lipton, S. A. Nature Neuroscience 2000, 3, 15-21; Jaffrey, S. R.; Erdjument-Bromage, H,; Ferris, C. D,; Tempst, P.; Snyder, S. H. Nature Cell Biology 2001, 3, 193-197; Matthews, J. R.; Botting, C, H.; Panico, M.; Morris, H. R.; Hay, R. T. Nucleic Acids Research 1996, 24, 2236-2242). Cerebral ischemia/reperfusion results in nitrosative and oxidative stress, and hence the production of NO and reactive oxygen species (ROS) (Sato, S.; Tominaga, T.; Ohnishi, T.; Ohnishi, S. T. Brain Research 1994, 647, 91-96; Kumura, E.; Kosaka, H.; Shiga, T.; Yoshimine, T.; Hayakawa, T. Journal of Cerebral Blood Plow & Metabolism 1994, 14, 487-491). Additionally, increases in NO and ROS have been reported in tissue culture models and rodent models of multiple sclerosis, HIY-associated dementia, and glaucoma, among other neurodegenerative disorders (Kaul, M.; Garden, G. A.; Lipton, S A. Nature 2001, 410, 988-994; Bukrinsky, M. L; Nottet, H. S. L. M.; Schmidtmayerova, H.; Dubrovsky, L.; Flanagan, C. R.; Mullins, M. E,; Lipton, S. A.; Gendelman, H. E. J. Exp. Med. 1995, 181, 735-745; Adamson, D. C.; McArthur, J. C; Dawson, T M.; Dawson, V. L. Molecular Medicine 1999, 5, 98-109; Adamson, D. C.; Kopnisky, K. L.; Dawson, T. M.; Dawson, V. L. Journal of Neuroscience 1999, 19, 64-71; Adamson, D. C.; Wildemann, B.; Sasaki, M.; Glass, J. D.; McArthur, J, C; Christov, V. L; Dawson, T., M.; Dawson, V. L. Science 1996, 274, 1917-21; Dreyer, E. B.; Zurakowski, D,; Gorla, M.; Vorwerk, C. K.; Lipton, S. A. Invest Opthalmol. Vis. Sci. 1999, 40, 983-989; Lipton, S, A.; Gendelman, H. E. N Engl J Med 1995, 332, 934-940).

The regulation of protein function by S-nitrosylation has led to the proposal that nitrosothiols function as posttranslational modifications analogous to phosphorylation or acetylation. Although the factors governing cysteine reactivity towards nitrosylating agents are not completely understood, some features include basic and acidic residues flanking the reactive cysteine, either in linear sequence or as a consequence of the three-dimensional organization of the protein, which catalyze the nitrosylation and denitrosylation steps (Stamler, J. S.; Toone, E. J.; Lipton, S. A,; Sucher, N. J. Neuron 1997, 18, 691-696). In proMMPs, a glutamate (E402 in MMP-9) is located ˜2.8 Å from the cysteine sulfur (Morgunova, E.; Tuuttila, A.; Bergmann, U.; Isupov, M.; Lindqvist, Y.; Schneider, G.; Tryggvason, K. Science 1999, 284, 1667-1670), and may act as a general base to remove the sulfhydryl proton (in the activated enzyme, this glutamate acts as abase to activate the Zn²⁺-bound water in a similar fashion). The reactivity of the cysteine sulfur may be further enhanced by its binding to the Zn²⁺ ion, which increases its nucleophilicity. Nitrosylation of this cysteine may reduce the nucleophilicity of the cysteine sulfur, weakening the bond to the zinc ion, and thus activating the enzyme. Therefore, S-nitrosylation may mechanistically trigger the cysteine switch to activate MMPs under pathophysiologically relevant conditions. Data presented herein demonstrate a novel extracellular proteolytic cascade in which S-nitrosylation leads to oxidative derivatization and hence activation of MMP-9 with consequent neuronal apoptosis. Moreover, this MMP derivatization pathway has been demonstrated to occur during stroke (Gu, Z.; Kaul, M.; Yan, B.; Kridel, S. J.; Cui, J.; Strongin, A.; Smith, J. W.; Liddington, R. C.; Lipton S. A. Science 2002, 297, 1186-1190).

Results and Discussion

A. MMPs are Activated by NO During Cerebral Ischemia

1. Co-Localization of MMP-9 and nNOS (Neuronal Nitric Oxide Synthase)

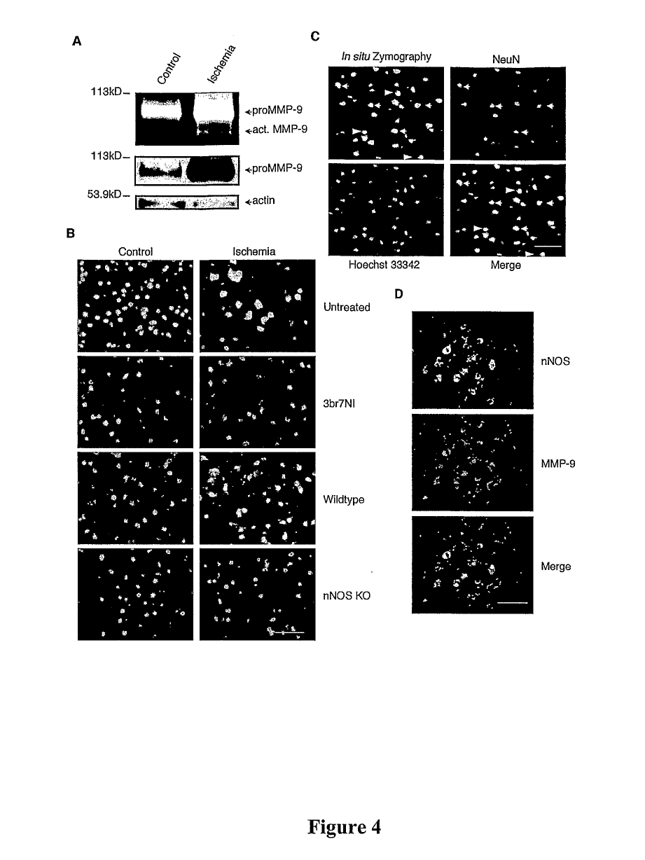

The association of MMP-9 and nNOS after focal cerebral ischemia/reperfusion in rodents was examined. Gelatin zymography revealed an increase in both the level of proMMP-9 and in MMP-9 activity in the ischemic hemisphere compared to the contralateral control hemisphere (FIG. 4A, top panel), Immunoblotting with an anti-MMP-9 antibody also showed increased MMP-9 levels in the ischemic hemisphere (FIG. 4A, middle panel). The slight decrease in actin in the damaged hemisphere may reflect cell loss (FIG. 4A, bottom panel). MMP-2 was not activated in this cerebral ischemia model in the rodent. Similar changes in MMP-9 have recently been reported after human embolic stroke (Montaner, J.; Alvarez-Sabin, J.; Molina, C; Angles, A.; Abilleira, S.; Arenillas, J.; Gonzalez, M. A.; Monasterio, J. Stroke 2001, 32, 1759-1766). In situ zymography and immunocytochemistry were used to examine the cellular localization of MMP-9 enzymatic activity. MMP activity was particularly elevated in ischemic brain parenchyma after ischemia and reperfusion (FIG. 4B, top panels). Moreover, activation of MMP was abrogated after stroke in nNOS knockout (KO) mice or in wild-type animals that were treated with the relatively specific nNOS inhibitor 3-bromo-7-nitroindazole (3br7NI; FIG. 4B, lower panels), suggesting that NO formation from nNOS was important in the activation of MMP-9 during cerebral ischemia. Previously, neuroprotection has been demonstrated under either of these conditions of NOS inhibition (Sato, S.; Tominaga, T.; Ohnishi, T.; Ohnishi, S. T. Brain Research 1994 647, 91-96; Kumura, E.; Kosaka, H.; Shiga, T.; Yoshimine, T.; Hayakawa, T. Journal of Cerebral Blood Flow & Metabolism 1994, 14, 487-491; Huang, Z.; Huang, P. L.; Panahian, N.; Dalkara, T.; Fishman, M. C; Moskowitz, M. A. Science 1994, 265, 1883-1885). In wild-type animals not treated with NOS inhibitors, immunocytochemistry revealed that many neurons in ischemic cortex manifested MMP activity (FIG. 4C> arrows). Substantial colocalization of MMP-9 and nNOS in the ischemic cortex was also observed (FIG. 4D). Hence, there is coincident production of NO and MMP-9 activity following ischemia and reperfusion,

2. S-Nitrosylation of MMPs

Whether MMP-9 could be S-nitrosylated, and thus activated by NO in vitro, was investigated. To eliminate effects of TIMP-1 binding to the hemopexin domain, which might interfere with catalysis and activation of MMP-9, recombinant proMMP-9 encoding the propeptide and catalytic domains of MMP-9 but lacking the hemopexin domain (R-proMMP-9) was initially used. R-proMMP-9, purified from conditioned medium of stably transfected human embryonic kidney 293 (HEK293) cells (Kridel, S. J.; Chen, E.; Kotra, L. P.; Howard, E. W.; Mobashery, S.; Smith, J. W. Journal of Biological Chemistry 2001, 276, 20572-20578), was incubated with the physiological NO donor S-nitrosocysteine (SNOC). The generation of S-nitrosothiol was detected by measurement of the fluorescent compound 2,3-naphthyltriazole (NAT) (Gu, Z. ibid., 2002). NAT is stoichiometrically converted from 2,3-diaminonaphthalene (DAN) by NO released from S-nitrosylated proteins and thus provides a quantitative measure of S-nitrosothiol formation (Wink, D. A.; Kim, S.; Coffin, D.; Cook, J. C; Vodovotz, Y.; Chistodoulou, D.; Jourd'heuil, D.; Grisham, M. B. Methods in Enzymology 1999, 301, 201-211). SNOC-treated R-proMMP-9 resulted in significant S-nitrosothiol formation (FIG. 5A). To insure that the S-nitrosothiol generated under these conditions represented S-nitroso-MMP-9 rather than residual SNOC, the stability of these S-nitrosothiols was examined at different incubation times. It was found that the S-nitrosylation product of SNOC-treated R-proMMP-9 was much more stable than SNOC alone; within 15 min of incubation, over 95% of the SNOC had decayed while over 80% of the S-nitroso-MMP-9 remained (FIG. 5B). This temporal separation provided the ability to distinguish SNOC from S-nitroso-MMP-9 in the fluorescent S-nitrosothiol assay.

To determine if S-nitrosylation of R-pro-MMP-9 resulted in its activation, the effects of the known exogenous MMP-9 activator, p-aminophenylmercuric acetate (APMA) with those of SNOC and another nitrosylating agent, acidified sodium nitrite, were compared. Gelatin zymography revealed that incubation with APMA, SNOC, or acidified sodium nitrite led to a partial conversion of the 53.5 kD R-proMMP-9 into the 41.2 kD activated form of MMP-9 (FIG. 5C); the respective masses were confirmed by mass spectrometry. The activation was inhibited in the presence of the poly-MMP-specific inhibitor GM6001. The activity of R-proMMP-9 incubated with APMA or SNOC was then compared by assaying the ability to cleave a synthetic fluorogenic peptide substrate (FIG. 5D). The initial velocity of R-proMMP-9 activation was 4.80 μM/hr by APMA compared to 0.88 μM/hr by SNOC. It was shown that SNOC led to activation of full-length MMP-9 in a fashion similar to that observed with MMP-9 that lacked the hemopexin domain (Gu, Z.; Kaul, M.; Yan, B.; Kridel, S. J.; Cui, J.; Strongin, A.; Smith, J. W.; Liddington, R. C; Lipton S. A. Science 2002, 297, 1186-1190). Taken together, these findings demonstrate that MMP-9 can undergo S-nitrosylation, and furthermore show for the first time that NO can directly promote activation of MMP-9.

3. Effect of NO-Activated MMPs on Neuronal Apoptosis in Culture

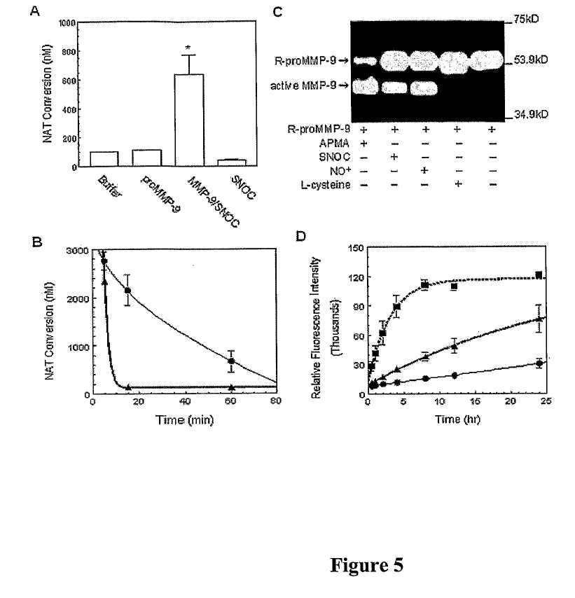

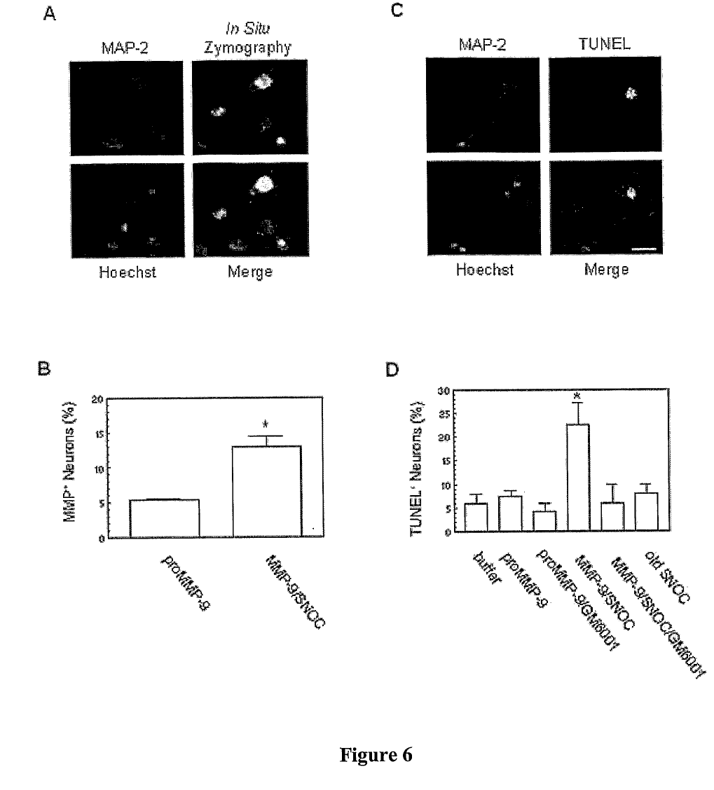

Effects of NO-activated MMP-9 on neuronal cell apoptosis in cerebrocortical cultures were evaluated. MMP activity was assessed by in situ zymography, neurons were identified by immunoreactivity for microtubule-associated protein-2 (MAP-2), and nuclear morphology was monitored with Hoechst dye 33342 (FIG. 6A). The percentage of neurons exhibiting MMP activity significantly increased after exposure to R-proMMP-9 preactivated with SNOC compared to R-proMMP-9 alone (FIG. 6B). SNOC, from which NO was dissipated, did not activate R-proMMP-9 and did not increase the percentage of neurons exhibiting MMP activity. Additionally, 18 hours after exposure to SNOC-activated R-proMMP-9, apoptotic neurons were assessed by staining with anti-MAP-2 and terminal-deoxynucleotidyl-transferase-mediated dUTP nick-end labeling (TUNEL; green) in conjunction with condensed nuclear morphology assessed with Hoechst 33342 (FIG. 6C). For these experiments, R-proMMP-9 was preexposed and hence preactivated by SNOC; NO had already been released from SNOC by the time the cultures were incubated with the activated MMP, as evidenced by measurement with an NO-sensitive electrode (WPI, Sarasota, Fla.) (Lipton, S. A.; Choi, Y. B.; Pan, Z. H.; Lei, S. Z.; Chen, H. S.; Sucher, N. J.; Loscalzo, J.; Singel, D. J.; Stamler, J. S. Nature 1993, 364, 626-632). Hence, direct release of NO from SNOC or the formation of peroxynitrite (ONOO⁻) due to the release of NO from SNOC and subsequent reaction with superoxide anion (O₂⁻) could not have accounted for the observed neuronal apoptosis (Lipton, S. A.; Choi, Y. B.; Pan, Z. H.; Lei, S. Z.; Chen, H. S.; Sucher, N. J.; Loscalzo, J.; Singel, D. J.; Stamler, J. S, Nature 1993, 364, 626-632). NO-activated MMP-9 resulted in significantly increased neuronal apoptosis, whereas treatment with the MMP inhibitor GM6001 blocked the neuronal cell death (FIG. 6D). Also, many neurons were observed coming up off the dish after exposure to NO-activated MMP-9. These results strongly suggest that even high levels of inactivated proMMP-9 protein do not have a deleterious effect on neurons. However, NO-triggered activation converts MMP-9 into a neurotoxin.

4. NO-Activation Leads to Further Stable Oxidation Products of MMPs (Sulfinic and Sulfonic Acid Derivatives) In Vitro and In Vivo

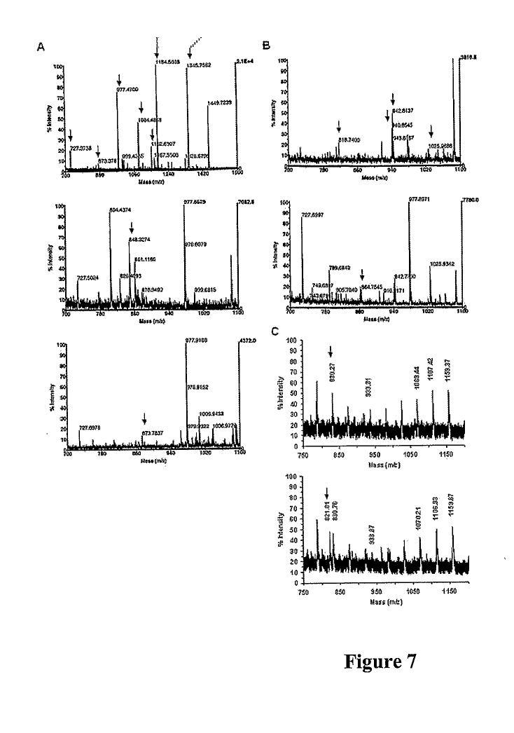

From the experiments using DAN to NAT conversion to show nitrosothiol generation, it was determined that S-nitroso-MMP-9 formation was associated with MMP-9 activation. However, nitrosothiols can be relatively short-lived and their reaction can be reversed by chemical reducing agents (Stamler, J. S.; Hausladen, A. Nature Structural Biology 1998, 5, 247-249). Alternatively, S-nitrosothiol formation could potentially lead to irreversible oxidative reactions that would result in the permanent activation of MMPs. It has been suggested that S-nitrosylation of proteins via NO may generally represent a signal transduction cascade, whereas subsequent oxidation via ROS can lead to irreversible modifications (Stamler, J. S.; Hausladen, A. Nature Structural Biology 1998, 5, 247-249). In fact, the observation that S-nitroso-MMP-9 was not stable over time and decayed within 1½ hours (FIG. 5B) while MMP-9 activation continued over the ensuing day (FIG. 5D), suggested that a more Long-lasting derivative of activated MMP-9 might be produced, especially in the presence of an oxidative insult. For example, it has been posited that reaction of S-nitrosylated enzymes (E) with ROS can result in the formation of irreversible sulfide (E-SO₂H) and sulfonic (E-SO₃H) acid derivatives (Stamler, J. S.; Hausladen, A. Nature Structural Biology 1998, 5, 247-249). To assess the possibility of these additional oxidative products and further identify the chemical nature of the NO-triggered-modification of MMP-9 responsible for activation, peptide mass fingerprinting was conducted (Gu, Z.; Kaul, M.; Yan, B.; Kridel, S. J.; Cui, J.; Strongin, A.; Smith, J. W.; Liddington, R. C.; Lipton S. A. Science 2002, 297, 1186-1190). Mass spectra were obtained after in-gel digestion of human R-proMMP-9 by trypsin using matrix-assisted laser desorption/ionization time-of-flight (MALDI-TOF) mass spectrometry. To perform chemical reduction and in-gel digests without disrupting the peptide fragments of interest in the MALDI-TOF analysis, free cysteines were first protected to avoid cleavage followed by uncontrolled disulfide formation. Therefore, cysteine was initially protected by iodoacetamide alkylation in the absence of SNOC exposure. Among eleven mass peaks, seven signature masses of human MMP-9 fragments that were virtually identical (<0.1% variation) to those predicted from theoretical tryptic fragments of MMP-9 deduced from the published amino acid sequences were observed (FIG. 7A, top panel). One of these peaks represented the region responsible for the cysteine switch in the propeptide domain of proMMP-9 (CGVPDLGR, 816 Da; SEQ ID NO: 1) that had been alkylated with iodoacetamide (57 Da), to yield a molecular mass of 873.4 Da (acet-CGVPDLGR; CGVPDLGR is represented by SEQ ID NO:1). In contrast, in vitro exposure of R-proMMP-9 to SNOC prior to attempted iodoacetamide alkylation yielded a tryptic fragment on MALDI-TOF analysis at 848.3 Da, indicating the addition of a stable 32 Da adduct to the propeptide domain CGVPDLGR (816 Da; SEQ ID NO:1) instead of alkylation (FIG. 7A, middle panel, n=4 experiments). Masking thiol groups by prior allylation with iodoacetamide before exposure to SNOC (yielding a mass of 873.8 Da) blocked the addition of the 32 Da adduct (FIG. 7A, bottom panel). In this chemical context, therefore, the 32 Da adduct represented addition of two oxygen molecules to the cysteine residue to form a sulfinic acid derivative (SO₂H-CGVPDLGR at 848 Da; CGVPDLGR is represented by SEQ ID NO; 1) (Stamler, J. S.; Hausladen, A. Nature Structural Biology 1998, 5, 247-249).

In another approach, which allows one to avoid the mandatory step of initial protection of the critical cysteine residue in the propeptide domain by alkylation, R-proMMP-9 was digested in solution under native conditions rather than in acrylamide gel slices. Using this method, a mass peak was found at 816.7 Da, representing the propeptide domain fragment CGVPDLGR (FIG. 7B, upper panel; SEQ ID NO:1). A 48 Da rather than a 32 Da shift in the mass spectrum of the 816 Da fragment after SNOC exposure was observed, yielding a peak at 864.8 Da, consistent with further oxidation to the sulfonic acid derivative (SO₃H-CGVPDLGR; FIG. 7B, n=3 experiments; CGVPDLGR is represented by SEQ ID NO: 1).

The results demonstrate that MMP-9 is activated during focal cerebral ischemia/reperfusion (stroke) and that inhibition of MMP activity ameliorates parenchymal brain damage (Romanic, A. M.; White, R. F.; Arleth, A. J.; Ohlstein, E. H.; Barone, F. C. Stroke 1998, 29, 1020-1030; Asahi, M.; Asahi, K.; Jung, J. C; del Zoppo, G. J.; Fini, M. E.; Lo, E. H. Journal of Cerebral Blood Flow & Metabolism 2000, 20, 1681-1689; Gasche, Y.; Fujimura, M,; Morita-Fujimura, Y.; Copin, J. C; Kawase, M.; Massengale, J.; Chan, P. H. Journal of Cerebral Blood Flow & Metabolism 1999, 19, 1020-1028). Furthermore, production of NO and ROS are known to occur in the early phase of reperfusion after focal ischemia (Sato, S.; Tominaga, T.; Ohnishi, T.; Ohnishi, S. T. Brain Research 1994, 647, 91-96; Kumura, E.; Kosaka, H.; Shiga, T.; Yoshimine, T.; Hayakawa, T. Journal of Cerebral Blood Flow & Metabolism 1994, 14, 487-491). Whether the oxidation products of MMP-9 that were encountered in vitro after S-nitrosylation were also present in vivo during focal ischemia/reperfusion was then investigated. Mass spectra of tryptic fragments from affinity-precipitated MMP-9 obtained from rat brain after a 2-hour middle cerebral artery (MCA) occlusion/15-min reperfusion injury or from the contralateral (control) side of the brain (n=12 animals) was examined. For these experiments, in-gel digestion with trypsin was performed because gel separation offered better protein resolution. MALDI-TOF analysis of specimens obtained from the control side of the brain revealed that after reduction and alleviation by iodoacetamide (57 Da), the rat propeptide domain fragment (CGVPDVGK, mass 774 Da; CGVPDVGK is represented by SEQ ID NO:3) yielded a peak at 830.3 Da, representing the alkylated fragment (acet-CGVPDVGK; CGVPDVGK is represented by SEQ ID NO:3) (FIG. 7C, top panel), hi contrast, on the side of the brain with the stroke, the propeptide domain was not as susceptible to reduction and alkylation as evidenced by the appearance of an additional peak indicating a propeptide tryptic fragment at 821.8 Da; this peak represented the addition of a 48 Da adduct in accord with sulfonic acid derivatization of the thiol group (SO₃H-CGVPDVGK; CGVPDVGK is represented by SEQ ID NO: 3) (FIG. 7C, bottom panel), and was similar to that found in vitro after NO activation of human MMP-9 (FIG. 7B). Additionally, MALDI-TOF mass fingerprinting analysis revealed that of the 19 cysteine residues present in MMP-9, only the cysteine in the propeptide domain that coordinates Zn²⁺ in the active site was irreversibly modified to a sulfinic (—SO₂H) or sulfonic (—SO₃H) acid in these experiments. These findings indicate that S-nitrosylation of this cysteine residue in the prodomain followed by further oxidation to a sulfinic or sulfonic acid derivative leads to activation of MMP-9. Unlike S-nitrosylation, these latter oxidative reactions are irreversible and therefore contribute to the pathophysiological activation of MMP-9, as found during cerebral ischemia. In additional experiments, similar activation pathways for MMPs in HIV-associated dementia, Alzheimer's Disease were found; this will probably generalize to most, if not all, disorders mentioned herein.

5. Crystal Structure Model of MMP Activation by NO and Formation of Stable Sulfinic and Sulfonic Acid Derivatives

One of the pathways proposed for oxidation of the nitrosylated cysteine is via hydrolysis to form a sulfenic acid: E-S—N═O+H2O→E-S—OH+HNO (Stamler, J. S.; Hausladen, A. Nature Structural Biology 1998, 5, 247-249). The MMP is set up to carry out hydrolysis of a peptide bond using an activated water molecule, and it is likely that the same machinery can be used to hydrolyze nitrosocysteine (FIG. 8). The sulfenic acid is labile and susceptible to facile oxidation to the stable sulfinic or sulfonic acid derivatives that were observed during MALDI-TOF peptide fingerprinting. Activation of the enzyme can occur prior to cleavage (Bannikov, G. A.; Karelina, T. V.; Collier, I. E.; Manner, B. L.; Goldberg, G. I. Journal of Biological Chemistry 2002, 277, 16022-16027) and with sulfinic or sulfonic acid modification since these derivatives were observed in the peptide analysis of pro-MMP-9.

6. Summary

Nitrosylation and subsequent oxidation of protein thiol in the prodomain of MMP-9 can lead to enzyme activation. It is likely that other, homologous MMPs, such as MMP-2, are activated in a similar manner. This series of reactions confers responsiveness of the extracellular matrix to nitrosative and oxidative stress. Such insults may be relevant to a number of pathophysiological conditions, including cerebral ischemia, HIV-associated dementia (HAD), glaucoma, multiple sclerosis, Alzheimer's diseases and other neurodegenerative disorders. Extracellular proteolytic cascades triggered by MMPs can disrupt the extracellular matrix, contribute to cell detachment, and lead to a form of apoptotic cell death known as anoikis, similar to that observed in neuronal cultures (Cardone, M. H.; Salvesen, G. S.; Widmann, C; Johnson, G.; Frisch, S. M. Cell 1997, 90, 315-323; Gu, Z.; Kaul, M.; Yan, B.; Kridel, S. J.; Cui, J.; Strongin, A.; Smith, J. W.; Liddington, R. C; Lipton S. A. Science 2002, 297, 1186-1190). The reactions described here are believed to represent the first case of combined nitrosative/oxidative activation of an enzyme that leads to apoptosis, and as such may represent a more general paradigm in molecular toxicology.

B. New MMP Inhibitors can Prevent the Extracellular Pathway to Neuronal Destruction

1. Studies Showing that Novel, Specific MMP-2,-9 Inhibitors Protect from Focal Cerebral Ischemia/Reperfusion.

hi the past few years, broad-spectrum MMP inhibitors have failed in clinical trials for stroke and other diseases for a variety of reasons, including toxicity of the chemical class that they represent (hydroxamates). Recently, more specific MMP-2/9 inhibitors (these inhibitors antagonize both MMP-2 and MMP-9) have been synthesized that are of fundamentally different chemical composition, and hence do not appear to be toxic in preliminary testing. Initially, the lead drug in this series, SB3CT, was supplied by the chemists at Wayne State University who originally made it (Brown, S.; Bernardo, M. M.; Li, Z. H.; Kotra, L. P.; Tanaka, Y.; Fridman, R.; Mobashery, S. Journal of American Chemical Society 2000, 122, 6799-800; Kleifeld, O.; Kotra, L. P.; Gervasi, D. C.; Brown, S.; Bernardo, M. M.; Fridman, R.; Mobashery, S.; Sagi. I. Journal of Biological Chemistry 2001, 276, 17125-17131; Bernardo, M. M.; Brown, S.; Li, Z. H.; Fridman, R.; Mobashery, S. Journal of Biological Chemistry 2002, 277, 11201-11207), but recently this drug has become commercially available through Chemicon, Inc. (Temecula, Calif.). The new drugs, represented by SB3CT, are fundamentally different from the previous hydroxamate MMP inhibitors that were bidentate (double coordinating) chelating ligands that bound to the MMP catalytic zinc ion. SB3CT has a sulfur atom that directly coordinates the MMP catalytic zinc in a monodentate manner to form a tetrahedral coordination (Brown, S.; Bernardo, M. M.; Li, Z. H.; Kotra, L. P.; Tanaka, Y.; Fridman, R.; Mobashery, S. Journal of American Chemical Society 2000, 122, 6799-800; Kleifeld, O.; Kotra, L. P.; Gervasi, D. C.; Brown, S.; Bernardo, M. M.; Fridman, R.; Mobashery, S.; Sagi. I. Journal of Biological Chemistry 2001, 276, 17125-17131; Bernardo, M. M.; Brown, S.; Li, Z. H.; Fridman, R.; Mobashery, S. Journal of Biological Chemistry 2002, 277, 11201-11207).

Initially, the new MMP inhibitors are tested to see if they can prevent S-nitrosylation MMP-2 and MMP-9 by the NO donor S-nitrosocysteine (SNOC) in vitro using recombinant MMPs and monitoring the chemical conversion of DAN to NAT (as in FIG. 5). Next, it is determined if the new MMP inhibitors can prevent activation of MMPs using gelatin zymography as well as by monitoring the cleavage rate of fluorogenic Substrate I Peptide (25 μM, Calbiochem, San Diego, Calif.; excitation wavelength, 280±1 nm; emission wavelength, 360±5 nm; also as in FIG. 5). Then, similar to the experiments described in FIG. 6, cultures of cerebrocortical neurons are used to test if the new MMP-2/9 inhibitors can prevent neuronal apoptosis in vitro due to NO-activation of MMP-9 and MMP-2. Finally, sulfonation of recombinant pro-MMP-2 and pro-MMP-9 is monitored following exposure to NO donors (such as SNOC) to determine if the new MMP inhibitors can prevent this chemical conversion using matrix-assisted laser desorption/ionization time-of-flight (MALDI-TOF) mass spectrometry, as in FIG. 7A. These studies will demonstrate if the new MMP inhibitors are capable of (i) preventing activation of recombinant MMP-2/9 by blocking S-nitrosylation and subsequent oxidation steps, and (ii) preventing neuronal cell death due to MMP-2/9.

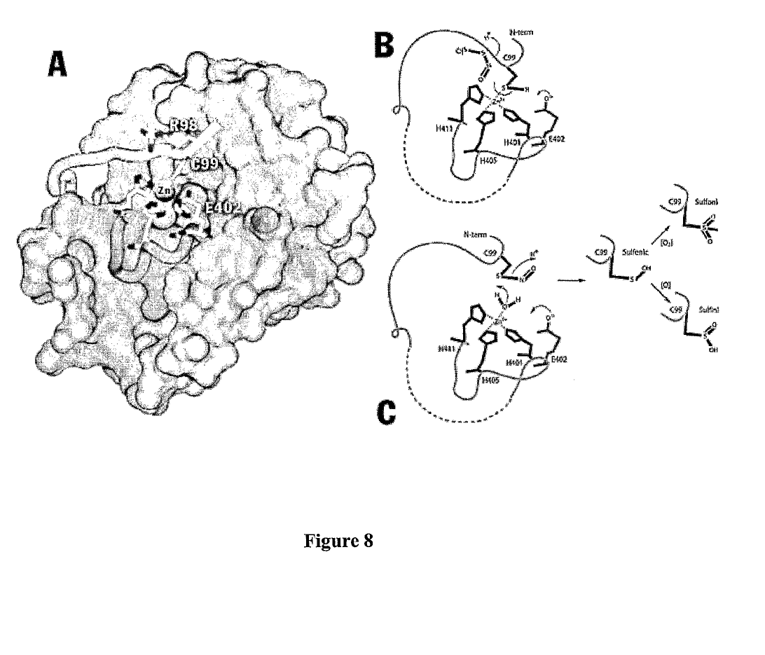

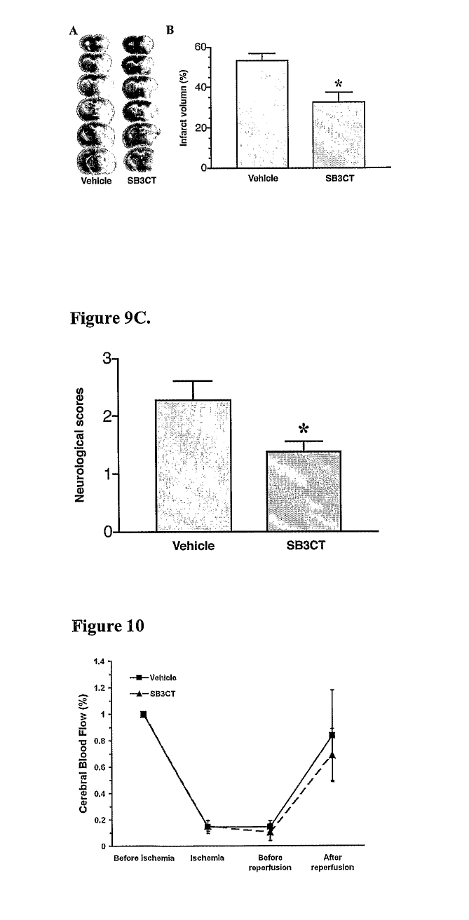

In a focal middle cerebral artery occlusion (MCAO)/reperfusion model in C57B1/6 mice, it was found that SB3CT protects the ischemic brain using TTC (2,3,5-triphenyltetrazolium chloride) staining (FIG. 9A, B). Additionally, neurobehavioral assessment of these mice was improved if pretreated with the MMP-2/9 inhibitor compared to control diluent solution (FIG. 9C).

As a control test, regional cerebral blood flow (rCBF), monitored by laser Doppler flowmetry, was unaffected by treatment with SB3CT compared to control (FIG. 10).

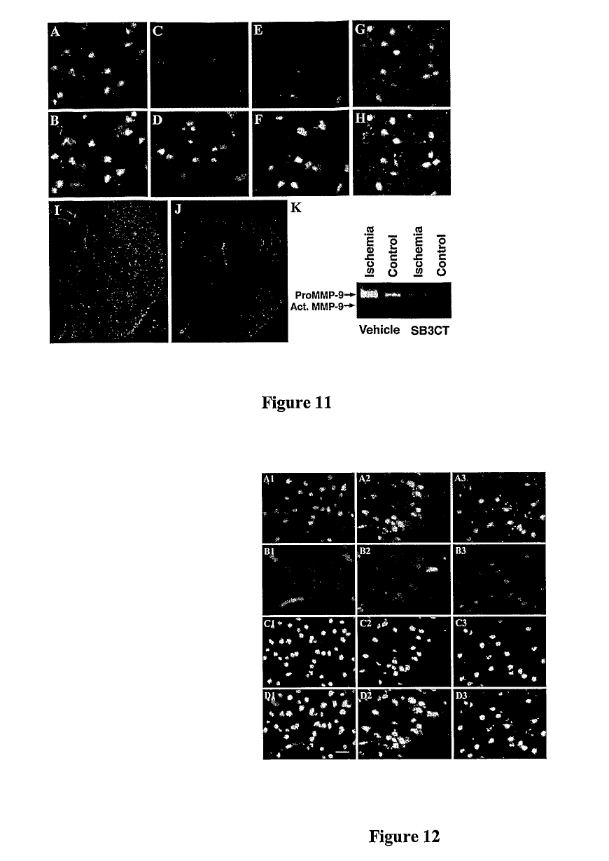

Additionally, it was found that the new MMP-2/9 inhibitor, SB3CT, similar to the previously available and more general inhibitors, GM6001 (also known as Ilomastat) and 1/10-phenanthroline, can prevent activation of MMP-9 by both in situ zymography (showing MMP activity associated with single neurons) and by gelatin zymography, reflecting activity of a brain lysate after MCAO/reperfusion (FIG. 11). Note that both proMMP-9 and the activated form of MMP-9 appear to be decreased after treatment with the MMP inhibitor. This is not unexpected because of positive feedback in the translation of MMPs based on their activity, as previously demonstrated,

C. Laminin is Degraded by MMPs During Ischemic-Related Damage

1. Degradation of Laminin by MMPs Underlies Neuronal Injury, at Least in Part, after Focal Cerebral Ischemia/Reperfusion

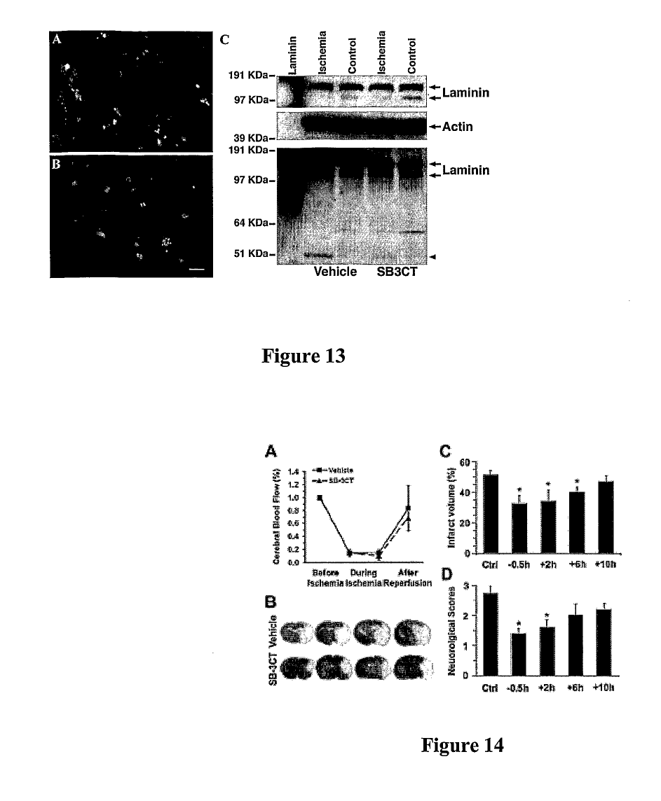

It has been previously shown that MMP activity is greatly increased in the ischemic cortex (Gu, Z.; Kaul, M.; Yan, B.; Kridel, S. J.; Cui, J.; Strongin, A.; Smith, J. W.; Liddington, R. C; Lipton S. A. Science 2002, 297, 1186-1190). Here, it is disclosed that MMP activity colocalizes with laminin (labeled with a poly-laminin polyclonal antibody (poly-Ln pAb) and apoptotic neuronal cell bodies (labeled by NeuN and TUNEL or Hoechst dye 33342 with condensed morphology) (FIG. 12). Moreover, it is herein demonstrated that the degradation of laminin by MMPs correlates with neuronal apoptosis (FIG. 13). While Eng Lo and colleagues could not establish that laminin was degraded during ischemia, they could also not completely rule it out (Asahi, M.; Wang, X.; Mori, T.; Sumii, T.; Jung, J.-C; Moskowitz, M. A.; Lo, E. The Journal of Neuroscience 2001, 21, 7724-7732). In fact, Sydney Strickland's laboratory has previously shown that tissue plasminogen activator (tPA) could contribute to ischemic damage via breakdown of hippocampal laminin (apparently the α5 subunit of laminin-10, which is composed α5, β1, γ1 subunits) (Chen, Z. L., Strickland, S., Cell (1997) 91:917-925; Indyck, J. A., Chen, Z. L., Tsirka, S. E., Strickland, S., Neuroscience (2003) 116:359-371; Wang, Y. F.; Tsirka, S. E.; Strickland, S.; Stieg, P. E.; Soriano, S. G.; Lipton, S. A. Nature Medicine 1998, 4, 228-231). While tPA is used as a clot buster and hence therapy for stroke, it was established that tPA could also directly contribute to neuronal damage. One postulated mechanism for this effect is that tPA is activating MMPs, which in turn degrade laminin.

### Example 9

### A Highly Specific Inhibitor of Matrix Metalloproteinase-9 Rescues Laminin from Proteolysis and Neurons from Apoptosis in Transient Focal Cerebral Ischemia

Introduction

Matrix metalloproteinases (MMPs) constitute a family of extracellular soluble or membrane-bound proteases that collectively can degrade or proteolytically modify essentially all of the extracellular matrix (ECM) main components, including collagens, laminin, and proteoglycans. A role of MMPs, MMP-9 in particular, has been suggested in the pathogenesis of neurologic disorders, including stroke (Rosenberg et al., 1996; Yong et al., 2001). MMP-9 is significantly elevated in humans after stroke (Castellanos et al., 2003; Horstmann et al., 2003), and MMP-2 levels have been reported to be acutely increased in the brains of baboons after stroke (Heo et al., 1999). A novel extracellular proteolytic cascade has recently been disclosed, in which S-nitrosylation (transfer of nitric oxide to a critical cysteine thiol group) and subsequent oxidation activates MMP-9 during cerebral ischemia, contributes to cortical neuronal apoptosis (Gu et al., 2002). Mice deficient in MMP-9 manifest a smaller cerebral infarct size; in addition, treatment with broad-spectrum MMP inhibitors or antibodies also reduces infarct size and prevents blood-brain barrier breakdown (Romanic et al., 1998; Asahi et al., 2001). Unfortunately, these broad-spectrum MMP inhibitors have significant systemic negative side effects.

Stroke ranks as the third leading cause of death in the United States. The only approved medical treatment for stroke is the administration of intravenous recombinant tissue plasminogen activator (tPA) within 3 h of stroke onset to restore cerebral blood flow (CBF) (National Institute of Neurological Disorders and Stroke rt-PA Stroke Study Group, 1995). The efficacy of tPA, however, is limited by its side effects, which include neurotoxicity and thrombolysis-associated hemorrhagic transformation (Tsirka et al., 1995; Wang et al., 1998; Castellanos et al., 2003). A recent report indicates that tPA also upregulates MMP-9 in the brain and that the subsequent matrix degradation contributes to brain damage (Wang et al., 2003).

In the past, efforts to ameliorate MMP-mediated brain damage with broad-spectrum MMP inhibitors (like sulfonamide or hydroxamate derivatives) have produced promising results in animal models of stroke (Asahi et al., 2000; Lapchak et al., 2000). However, the low specificity, and thus systemic toxicity, of hydroxamates and sulfonamides have precluded the use of these broad-spectrum inhibitors in clinical studies (Coussens et al., 2002; Overall and Lopez-Otin, 2002). These previous MMP inhibitors were designed to provide a bidentate (double coordinating) chelating ligand to the MMP catalytic zinc and thus are incapable of discriminating among the members of the MMP family and selectively targeting individual MMPs. Herein, a fundamentally different, “suicide type” thiirane MMP inhibitor, designated SB-3CT is described. SB-3CT is the first mechanism-based MMP inhibitor that is selective for the gelatinases MMP-2 and MMP-9 (Brown et al., 2000). SB-3CT coordinates the catalytic zinc ion, contributing to both slow binding and mechanism-based inhibition. This suicide type of inhibition is unique among MMP inhibitors developed to date (Brown et al., 2000; Kleifeld et al., 2001).

Herein, We took advantage of the specificity and selectivity of SB-3CT was taken advantage of to determine whether it could protect against brain damage in a mouse model of stroke without causing negative systemic side effects and further examine the molecular consequences of inhibiting MMP-9 proteolysis in the brain.

Materials and Methods

In vivo transient focal cerebral ischemia. Following a protocol approved by The Burnham Institute Animal Usage Committee, C57BL/6J mice weighing 25-30 g were housed in a 12 h light/dark cycle and permitted food and water intake ad libitum. These mice were subjected to transient focal cerebral ischemia under isoflurane anesthesia using a 6.0 monofilament suture to intraluminally occlude the right middle cerebral artery (MCA) for 2 h. The filament was then removed for reperfusion (R) for 24 h (Wang et al., 1998; Gu et al., 2002). A laser Doppler flowmeter (Perimed, North Royalton, Ohio) with the probe fixed on the skull surface (3 mm lateral to midline and 2 mm posterior to the bregma), located at the distal arterial supply of the middle cerebral artery, measured regional CBF (rCBF), as described previously (Wang et al., 1998). The initial reading of rCBF was assigned a value of 100%, and subsequent readings were expressed relative to this value. SB-3CT was designed as a highly selective, mechanism-based inhibitor to MMP-2 and MMP-9. The Ki values of MMP-2 and MMP-9 are in the nanomolar range, which are similar to the Ki values of endogenous TIMPs (tissue inhibitors of metalloproteinases). In contrast, the Ki values of SB-3CT against other MMPs (MMP-1, MMP-3, and MMP-7) are in the micromolar range (Brown et al., 2000). SB-3CT (25 mg/kg body weight) was injected intraperitoneally as a suspension in a vehicle solution (10% DMSO in normal saline). Mice were divided into four groups for administration of SB-3CT. One group was initially treated 30 min before ischemia, followed by a second injection immediately before reperfusion. The other three groups were treated at different time points after ischemia and received the first injection 2, 6, or 10 h after occlusion, followed by a second injection 3 h later. The control groups received only vehicle in each case. Mice were killed 24 h after reperfusion, and brains were dissected to prepare unfixed tissue OCT blocks for an in situ MMP gelatinolytic activity assay or storage at 80° C. for later analysis. Infarct volumes were quantified with NIH Image software (version 1.62) on 1.0-mm-thick coronal sections stained with 2,3,5-triphenyltetrazolium chloride (TTC) (Wang et al., 1998). To minimize the effect of brain edema, the infarct volume was determined by subtracting the volume of the contralateral noninfarcted hemisphere (left) from the ipsilateral hemisphere (right). The right femoral artery was cannulated to monitor blood pressure and sample arterial blood gases and glucose. Arterial blood pressure was continually recorded before ischemia, during ischemia, and at reperfusion with a blood-pressure transducer, a bridge amplifier, and a computerized data acquisition system (MacLabs 8s; ADInstruments, Castle Hill, New South Wales, Australia). Arterial blood gases and glucose were measured before ischemia and 15 min after reperfusion with a blood gas and glucose analyzer (Stat Profile Ultra C; Nova Biomedical, Waltham, Mass.).

Assessment of neurological deficits. After the MCA is occluded, an animal usually exhibits marked thorax twisting when suspended by the tail. Contralateral forepaw flexure is also usually observed. The severity of this neurological deficit (referred to as postural asymmetry) was assessed using a five-point scale (Wang et al., 1998; Asahi et al., 2001): 0, no postural asymmetry (animal reaches down with both forepaws); 1, failure to extend the contralateral forepaw; 2, twisting the entire body toward the contralateral side; 3, falling to the contralateral side; 4, inability to walk spontaneously.

Gelatin zymography and in situ zymography. The concentration and activation of MMP-9 or MMP-2 in brain homogenates were determined by gelatin zymography (Zhang and Gottschall, 1997; Gu et al., 2002). Brain tissues were homogenized in Tris-buffered saline (TBS), pH 7.6, containing 5 mM CaCl₂, 150 mM NaCl, 0.05% Brij 35, 0.02% NaN₃, 1% Triton X-100, 100 M PMSF, and a protein inhibitor cocktail (PIC; Roche Diagnostics, Mannheim, Germany) and centrifuged at 10,000 g for 30 min. Aliquots of the supernatant, containing 1.5 mg of protein, were subjected to affinity precipitation with gelatin-conjugated Sepharose beads (Gelatin Sepharose 4B; Amersham Biosciences, Piscataway, N.J.). The bound material was released from the beads in 1% SDS, and the samples were analyzed by electrophoresis in a 10% gelatin zymogram gel (Invitrogen, Carlsbad, Calif.). For in situ zymography, 8-m-thick sections were cut from OCT-embedded fresh mouse brain and incubated overnight at 37° C. with 10 g/ml DQ-gelatin-FITC (DQ-gel; Molecular Probes, Eugene, Oreg.) in TBS (Gu et al., 2002).

Ex vivo laminin degradation and Western blots. Brain lysates (50 mg of total protein in TBS with 0.05% Brij 35) were incubated overnight at 37° C. with activated MMP-9, latent preform of MMP-9 (proMMP-9), or the catalytic domain of membrane type 1 (MT1)-MMP. The digested samples were subjected to electrophoresis on a bis-Tris 4-12% acrylamide gradient gel (Invitrogen). Laminin and MMP-9 were analyzed by immunoblotting using rabbit pan-laminin (pan-Ln) and MMP-9 antibodies (from Sigma (St. Louis, Mo.) and Calbiochem (La Jolla, Calif.), respectively), followed by peroxidase-conjugated anti-rabbit IgG antibody. Peroxidase was visualized by enhanced chemiluminescence (Amersham Biosciences).

Immunohistochemistry. Brain sections were immunostained with antibodies to NeuN (a well known neuronal marker; Chemicon, Temecula, Calif.), pan-Ln, laminin-2 (generated by Dr. Eva Engvall, The Burnham Institute, La Jolla, Calif.), and -5 (from Dr. Jeffrey Minor, Washington University, St. Louis, Mo.) and visualized with fluorescent chromatin conjugated secondary antibodies (Jackson ImmunoResearch, West Grove, Pa.) (Indyk et al., 2003).

Apoptosis detection. After intracardiac perfusion with 4% paraformaldehyde, brains were dissected, 16 coronal sections were cut, and apoptotic-like cell nuclei were detected by terminal deoxynucleotidyl transferase-mediated dUTP nick end labeling (TUNEL; Roche Diagnostics) and Hoechst dye 33342 (Sigma) to identify characteristic condensed apoptotic bodies (Gu et al., 2002).

Intracortical infusions. Mice were anesthetized as described above and placed in a stereotaxic apparatus. An Alzet micro-osmotic pump (Durect, Cupertino, Calif.), containing 100 1 of 1% BSA in PBS, normal rabbit preimmune serum, or affinity-purified rabbit pan-Ln antibodies (0.25 mg/ml in 1% BSA/PBS; Sigma) was then placed subcutaneously on the backs of the animals (Chen and Strickland, 1997). A brain infusion cannula connected to the pump was positioned at the following coordinates: bregma, 1.0 mm; mediolateral, 1.5 mm; and dorsoventral, 1.6 mm. The infusion rate was 0.5 l/h, and the pump was allowed to infuse the designated solution for 2 d before MCA occlusion (MCAO). The mice were killed 1 d after MCA occlusion/reperfusion; the brains were processed for histochemical staining with cresyl violet and acid fuchsin and assessed for neuronal survival.

Results

SB-3CT Protects Against Brain Damage and Ameliorates Neurological Deficits after Transient Focal Cerebral Ischemia.

As a model of focal ischemia in C57BL/6 mice, the right middle cerebral artery was occluded for 2 h, followed by 24 h of reperfusion (MCAO/R) (Wang et al., 1998). To ensure successful placement of the intraluminal suture for occlusion and subsequent reperfusion, the rCBF in the area of the right middle cerebral artery was monitored in all animals. All mice subjected toMCAO/Rmet the criteria that the rCBF was reduced to 25% of the baseline during ischemia and recovered to 50% of the baseline during reperfusion (FIG. 14A). Infarct volume was measured by staining brain sections with TTC. Animals treated with vehicle alone (10% DSMO in normal saline) developed extensive brain damage in the ischemic cortex, the striatum, and the hippocampus (FIG. 14B). The mean volume of the infarction was 55% of the total hemisphere after accounting for brain edema (Wang et al., 1998). Cresyl violet staining and Fluoro-Jade B staining, which are specific for degenerating cells, confirmed neuronal cell death in the ischemic region (data not shown). Both preischemic and 2 h postischemic injections of SB-3CT reduced infarct volume to 30% of control. Administration of this thiirane MMP inhibitor 6 h after ischemia was still potently protective histologically, with an infarct volume of 40% compared with 55% in vehicle-treated animals, thereby providing significant protection against brain ischemia (FIG. 14C). The severity of the neurobehavioral deficits of these mice was also evaluated 24 h after reperfusion, quantifying the findings using a five-point scale (see Materials and Methods) (Asahi et al., 2001). Mice treated with SB-3CT either before ischemia or 2 h after ischemia manifested significant improvement in behavioral scores compared with vehicle-treated controls (FIG. 14D). Treatment with SB-3CT did not affect physiological parameters including arterial blood gases and glucose compared with vehicle-treated controls (Table 2).

TABLE 2
Physiological Variables

| | Before | | During | | After | |
|---|---|---|---|---|---|---|
| | MCAO | | MCAO | | MCAO | |
| | Vehicle | SB-3CT | Vehicle | SB-3CT | Vehicle | SB-3CT |
| MABP | 76.17 ± 4.65 | 78.68 ± 3.17 | 76.22 ± 4.90 | 79.49 ± 3.48 | 76.91 ± 4.43 | 76.12 ± 2.89 |
| (mmHg) | | | | | | |
| pH | 7.274 ± 0.020 | 7.313 ± 0.060 | 7.177 ± 0.078 | 7.106 ± 0.025 | 7.139 ± 0.078 | 7.170 ± 0.027 |
| PCO₂ | 37.27 ± 1.28 | 40.70 ± 7.828 | 50.50 ± 11.33 | 44.95 ± 14.55 | 51.93 ± 10.58 | 43.90 ± 5.28 |
| (mmHg) | | | | | | |
| PO₂ | 252.8 ± 6.7 | 230.8 ± 31.7 | 259.2 ± 21.9 | 214.5 ± 10.3 | 242.5 ± 6.0 | 245.4 ± 24.0 |
| (mmHg) | | | | | | |
| Na⁺ | 154.9 ± 1.1 | 155.7 ± 1.8 | 158.4 ± 2.4 | 157.20 ± 0.5 | 158.7 ± 2.6 | 157.7 ± 2.0 |
| (mmol/L) | | | | | | |
| Ca²⁺ | 1.113 ± 0.003 | 1.150 ± 0.021 | 1.133 ± 0.014 | 1.117 ± 0.081 | 1.157 ± 0.023 | 1.100 ± 0.074 |
| (mmol/L) | | | | | | |
| Glucose | 147.7 ± 7.5 | 160.7 ± 13.2 | 148.0 ± 11.5 | 162.70 ± 13.3 | 147.7 ± 11.7 | 154.3 ± 12.6 |
| (mg/dl) | | | | | | |

Note that no significant changes in the physiological values of blood gases and electrolytes in animals with or without MCAO were observed. Blood was drawn 5 min. before ischemia, 15 min. after reperfusion. Data are mean ± SEM (n = 5). MABP, Mean artificial blood pressure.

SB-3CT Inhibits MMP-9 Activity after Transient Focal Cerebral Ischemia.

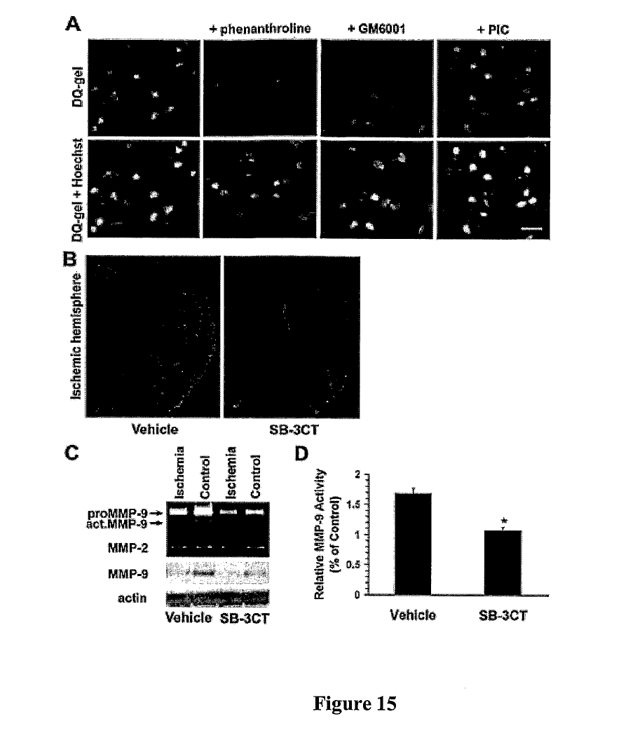

To determine whether administration of SB-3CT inhibited total gelatinolytic MMP activity after ischemia, in situ zymography together with Hoechst dye counter staining was used in the mouse stroke model. Gelatinolytic MMP activity in the ischemic cortex of samples treated with the broad-spectrum MMP inhibitors 1,10-phenanthroline or GM6001 (Ilomastat; Chemicon) versus control were compared. In the ischemic cortex, the broad spectrum MMP inhibitors abrogated gelatinolytic MMP activity. In contrast, a non-MMP PIC had no effect on gelatinolytic activity (FIG. 15A). Deconvolution microscopy revealed that SB-3CT also significantly reduced the gelatinolytic activity in the ischemic region compared with vehicle-treated controls (FIG. 15B). Consistent with these findings, gelatin zymography and Western blot assays demonstrated that the levels of active MMP-9 significantly decreased in ischemic brains after SB-3CT treatment (FIG. 15C-D). As noted previously, proMMP-9 levels increased after stroke (Rosenberg et al., 1996; Romanic et al., 1998; Asahi et al., 2000; Gu et al., 2002). Intriguingly, the level of proMMP-9 also decreased after SB-3CT treatment. The reason is readily explained by a positive feedback mechanism that links MMP-9 activity to the efficacy of MMP-9 gene transcription, with low levels of active MMP-9 resulting in less transcription of proMMP-9 (Opdenakker et al., 2001). Under the conditions present herein, changes in MMP-2 levels after stroke in these rodents was not observed (Gu et al., 2002).

Increased MMP Gelatinolytic Activity is Spatially Associated with Neuronal Laminin after Transient Focal Cerebral Ischemia.

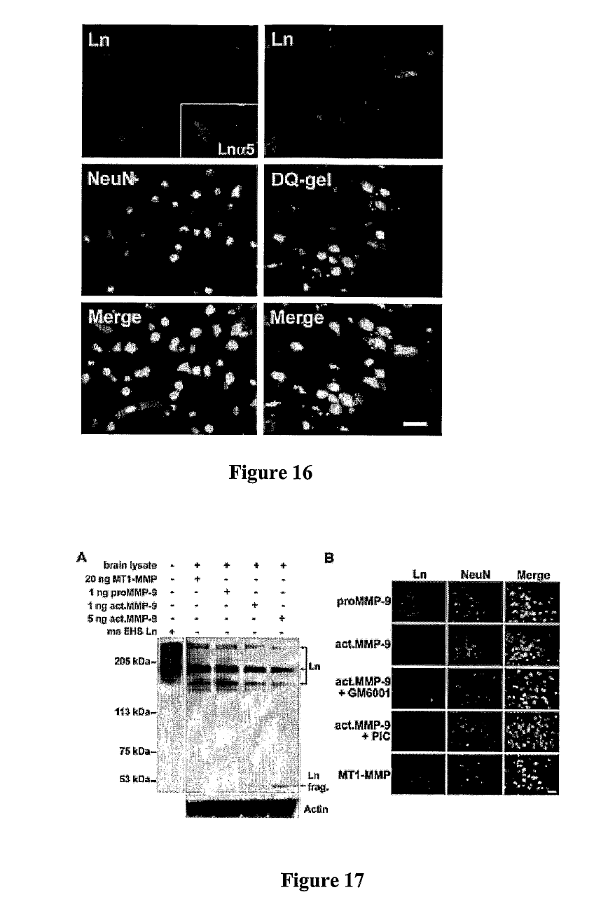

It has been shown that activated MMP-9 directly induces neuronal apoptosis both in vitro and in vivo after focal cerebral ischemia/reperfusion (Gu et al., 2002). An additional recent report states that MMP-9contributes to delayed neuronal cell death in the hippocampus after transient global ischemia (Lee et al., 2004). Although several reports (Asahi et al., 2001; Castellanos et al, 2003; Chen et al., 2003; Hamann, 2003; Horstmann et al., 2003) suggest that basement membrane proteins are involved in an MMP-9 proteolytic pathway, it is unclear how MMP substrates contribute to neuronal cell death. To identify potential targets of MMP-9 proteolysis in ischemic cortex, several basement membrane proteins, including laminin, were examined by immunohistochemistry. Pan-Ln antibody staining, followed by deconvolution microscopy, detected high immunoreactivity in proximity to NeuN-positive cells (FIG. 16, left panels), and microvascular structures. Excess laminin (purified from the mouse Engelbreth-Holm-Swarm sarcoma) completely blocked anti-Ln immunostaining in these brain sections (data not shown), indicating the high specificity of the pan-Ln immunoreactivity. This neuronal laminin immunoreactivity was confirmed by immunostaining with a polyclonal antibody against the neuron-associated-5 laminin subunit (FIG. 16, top left, inset). Ln immunoreactivity was also detected in regions adjacent to laminin-2 subunit-positive microvascular structures (data not shown). According to these immunostaining data and in situ zymography, gelatinolytic activity was primarily colocalized with Ln-positive neurons in the ischemic cortex, within 2 h of reperfusion (FIG. 16, right panels). These data suggest colocalization of MMP-9 activity with neuronal laminin in the early stages of brain damage after transient ischemia.

Exogenous MMP-9 Degrades Laminin in Mouse Brain.

To corroborate these observations, it was tested whether purified activated MMP-9 can cleave laminin. For this purpose, activated MMP-9 was coincubated with brain lysates and followed lamimn cleavage by analyzing digested samples with SDS-PAGE and Western blotting. It was determined that MMP-9, in a dose-dependent manner, cleaved laminin subunits (FIG. 17A, top bands) to generate a 51 kDa fragment. As controls, latent proMMP-9 or catalytically active MT1-MMP did not degrade laminin (FIG. 17A,B). A broad-spectrum MMP inhibitor, GM6001, unlike a cocktail of non-MMP protease inhibitors, inhibited MMP-9 proteolysis of neuronal laminin (FIG. 17B). Together, the data indicate that MMP-9 can cleave laminin on the neuronal surface.

MMP-9 Activation is Essential for Degradation of Laminin after Transient Focal Cerebral Ischemia.

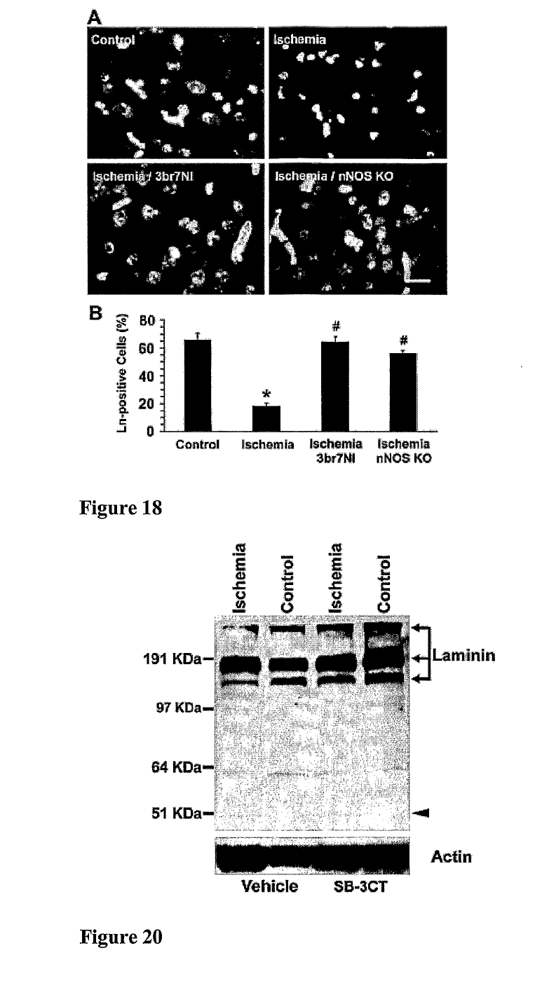

Previously, it was reported (Gu et al., 2002) that S-nitrosylation of MMP-9 leads to its activation and that increased MMP-9 activity in the ischemic cortex does not occur in neuronal NOS (nNOS) knock-out (KO) mice or in wild-type mice treated with the specific nNOS inhibitor 3-bromo-7-nitroindazole. Herein, blockage of laminin degradation was demonstrated in the ischemic cortex during stroke in nNOS KO mice and in wild-type mice treated with the specific nNOS inhibitor (FIG. 18). These new results provide evidence for a link between MMP-9 activation by S-nitrosylation and laminin cleavage.

Increased MMP Gelatinolytic Activity Induces Laminin Degradation Before Apoptotic Cell Death in the Ischemic Cortex.

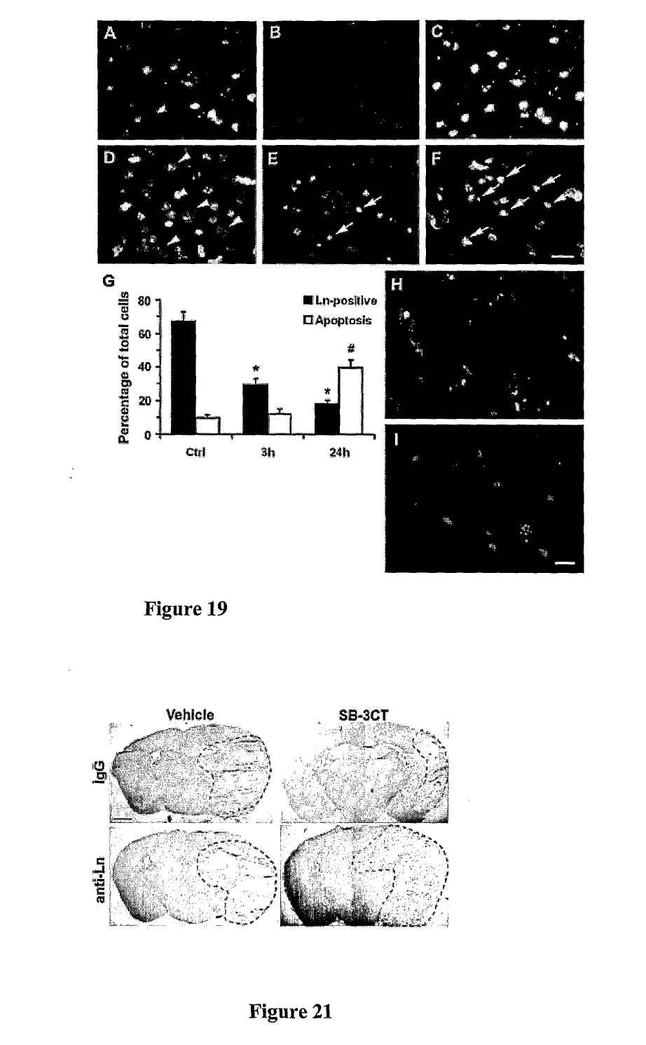

The time course of laminin degradation and apoptotic cell death in the ischemic cortex was examined. Increased gelatinolytic activity was associated with apoptotic cells 24 h after reperfusion (FIG. 19A-C). Decreased laminin immunoreactivity with moderate cell death 3 h after reperfusion and increasing apoptotic cell death 24 h later was also found (FIG. 19D-I). These results indicate that laminin degradation in the ischemic cortex occurs before cell death and after MMP activation.

SB-3CT Attenuates Laminin Degradation after Transient Focal Cerebral Ischemia.

The effects of the specific MMP-9/MMP-2 inhibitor SB-3CT on MMP-mediated proteolysis of laminin in vivo in the mouse stroke model was also examined. Western blots of brain extracts demonstrated partial degradation of laminin to a 51 kDa fragment in the ischemic hemisphere after transient MCAO (FIG. 20). Degradation of laminin and formation of the 51 kDa laminin fragment were reduced in the ischemic hemisphere of mice injected with SB-3CT, indicating that specific gelatinase inhibition significantly protected laminin from proteolysis.

Disruption of Laminin-Cell Surface Interactions with Anti-Laminin Antibody Increases Neuronal Death in the Ischemic Mouse Brain Treated with SB-3CT.

To determine causality between laminin degradation and neuronal cell death in vivo, a pan-laminin antibody was infused into the brain. If laminin degradation contributes to neuronal cell death, this procedure should increase neuronal vulnerability during MCAO despite treatment with SB-3CT. First, we brain infusions were performed with laminin antibody or control IgG into normal animals. No significant cellular damage was found, except for minor changes at the site of infusion (data not shown). However, if anti-laminin antibody was infused into the brains of ischemic animals treated with SB-3CT, significant damage and neuronal cell death in the ischemic regions, including the cortex, hippocampus, and striatum was found. This finding suggested that the anti-laminin antibody had abrogated the neuroprotective effect of SB-3CT; as a control, normal serum did not alter the protective effect of SB-3CT (FIG. 21),

Discussion

MMPs have been implicated in the pathogenesis of brain injury after ischemia and a number of neurodegenerative disorders (Rosenberg et al., 1996; Yong et al., 2001). After various insults, MMPs, especially MMP-9 and MMP-2, are upregulated and lead to neuronal cell death and/or hemorrhagic consequences because of neurovascular matrix degradation (Heo et al., 1999; Asahi et al., 2001; Gu et al., 2002; Horstmann et al., 2003). Li the mouse brain, MMP-9 appears to play a dominant role, because MMP-9 knock-out mice are relatively protected from ischemic and traumatic damage (Asahi et al., 2001). Broadspectrum pharmacological inhibitors of MMPs significantly reduce brain damage after insults in animal models (Romanic et al., 1998; Asahi et al., 2000). However, previous human clinical trials with MMP inhibitors, representing hydroxamate derivatives, failed because of side effects attributed, at least in part, to their lack of specificity (Coussens et al., 2002; Overall and Lopez-Otin, 2002). The results demonstrate that a new chemical class of MMP inhibitors, represented by thiirane derivative SB-3CT, potently decreases brain damage and can extend the window of therapeutic intervention to 6 h after insult in animal models of cerebral ischemia/reperfusion. This class of drugs represents a more specific form of MMP antagonist, targeting only MMP-9 and MMP-2, and appears to be well tolerated, at least in our animal models.

Additionally, the results show that the proteolysis of laminin by MMP-9 follows cerebral ischemia and contributes to neuronal apoptosis. These results build on the previous work that identified the molecular mechanism whereby nitric oxide (NO) activates MMP-9 in the brain (Gu et al., 2002). MMP-9 may arise from different cell types, including neutrophils and macrophages, which are known to migrate into the brain after damage to the blood-brain barrier because of ischemia/reperfusion injury (Yang et ah, 2001). Although Lo and colleagues (Asahi et al., 2001) could not unambiguously demonstrate degradation of lamimn in the ischemic brain, they could not rule out this possibility. The results support a model in which production of proMMP-9, and subsequent activation by NO (Gu et al., 2002) or tPA (Zhao et al., 2004), leads to laminin cleavage and resulting neuronal cell death. In fact, the effect of MMP-9 is functionally similar to that of tPA, which is also known to contribute to laminin degradation (Chen and Strickland, 1997; Wang et al., 1998; Indyk et al., 2003). This point underscores the fact that tPA, which is frequently used as a clot buster and hence as a therapy for stroke, can also contribute to neuronal cell death (Wang et al., 1998). Nonetheless, specific inhibition of MMP-9 should be able to prevent the dire consequences of excessive activation of this gelatinase MMP either by NO or tPA.

Previously, it had been demonstrated that ECM proteins such as laminin are important for cell survival and prevention of apoptosis, representing a form of cell death known as anoikis, in which cells detach from their matrix (Frisch and Francis, 1994). If cells are prohibited from interacting with the ECM, their viability is thus impaired. The laminin antibody that we used disrupts cell-laminin interactions and can therefore contribute to neuronal cell death (Chen and Strickland, 1997). These data suggest that laminin serves as a cell-survival factor in this system. The anti-laminin neutralizing antibody was used in this case to show that the effect of laminin disruption was downstream to the action of the SB-3CT compound, because treatment with SB-3CT was unable to rescue neurons from damage initiated by the antilaminin antibody.

In summary, it was shown that SB-3CT, a mechanism-based and selective gelatinase inhibitor, provides significant protection against brain damage in an experimental model of focal cerebral ischemia, consistent with a role for MMP-9 in laminin degradation and neuronal cell death in stroke. It is conclude that targeting MMP-9 in stroke patients is a highly promising therapeutic approach. The use of novel and selective MMP inhibitors such as SB-3CT or its derivatives, which exhibit selective inhibition of MMP-9/MMP-2 gelatinases, should minimize the undesired side effects caused by broad spectrum MMP inhibitors in previous clinical trials. This study is the first to demonstrate a significant pharmacological benefit of a new type of synthetic MMP inhibitor in an animal model of stroke. These results hold promise for the successful application of SB-3CT derivatives to stroke patients.

### BIBLIOGRAPHY

Adamson, D. C., Kopnisky, K. L., Dawson, T. M., and Dawson, V. L., Mechanisms and structural determinants of HIV-1 coat protein, gp41-induced neurotoxicity, Journal of Neuroscience, 19, 64-71 (1999).
Adamson, D. C., McArthur, J. C, Dawson, T. M., and Dawson, V. L., Rate and severity of HIV-associated dementia (HAD): correlations with gp41 and iNOS, Molecular Medicine, 5, 98-109, (1999).
Adamson, D, C, Wildemann, B., Sasaki, M., Glass, J. D., McArthur, J. C, Christov, V. L, Dawson, T., M., and Dawson, V. L., Immunologic NO synthase: elevation in severe AIDS dementia and induction by HIV-1 gp41, Science, 274, 1917-21 (1996).
Ankarcrona M, Dypbukt J M, Bonfoco E, Zhivotovsky B, Orrenius S, Lipton S A, Nicotera P. Glutamate-induced neuronal death: A succession of necrosis or apoptosis depending on mitochondrial integrity. Neuron 1995; 15:961-973.
Asahi M, Asahi K, Jung J C, del Zoppo G J, Fini M E, Lo E H (2000) Role for matrix metalloproteinase 9 after focal cerebral ischemia: effects of gene knockout and enzyme inhibition with BB-94. J Cereb Blood Flow Metab 20:1681-1689.
Asahi M, Wang X, Mori T, Sumii T, Jung J C, Moskowitz M A, Fini M E, Lo E H (2001) Effects of matrix metalloproteinase-9 gene knock-out on the proteolysis of blood-brain barrier and white matter components after cerebral ischemia. J Neurosci 21:7724-7732.
Asahi, M., Asahi, K., Jung, J. C, del Zoppo, G. J., Fini, M. E., and Lo, E, H., Role for matrix metalloproteinase 9 after focal cerebral ischemia: effects of gene knockout and enzyme inhibition with BB-94, Journal of Cerebral Blood Flow & Metabolism, 20, 1681-1689 (2000).
Bai G, Lipton S A, Aberrant RNA splicing in sporadic amyotrophic lateral sclerosis. Neuron 1998; 20:363-366.
Bannikov, G. A., Karelina, T. V., Collier, I. E., Manner, B. L., and Goldberg, G. I., Substrate binding of gelatinase B induces its enzymatic activity in the presence of intact propeptide, Journal of Biological Chemistry, 277, 16022-16027 (2002).
Becker, K., Savvides, S. N., Keese, M., Schirmer, R. H., and Karplus, P. A., Enzyme inactivation through sulfhydryl oxidation by physiologic NO-carriers, Nature Structural Biology, 5, 267-271 (1998).
Bernardo, M. M., Brown, S., Li, Z. H., Fridman, R., and Mobashery, S., Design, synthesis, and characterization of potent, slow-binding inhibitors that are selective for gelatinases, Journal of Biological Chemistry, 277, 11201-11207 (2002).

Bonfoco E, Ankarcrona M, Krainc D, Nicotera P, Lipton S A. Techniques for distinguishing apoptosis from necrosis in cerebrocortical and cerebellar neurons. In: Neuromethods: Apoptosis Techniques and Protocols, Poirier J, ed, Totowa, N.J.: Humana Press, 1997. pp. 237-253.
Bonfoco E, Krainc D, Ankarcrona M, Nicotera P, Lipton S A. Apoptosis and necrosis: two distinct events induced respectively by mild and intense insults with NMDA or nitric oxide/superoxide in cortical cell cultures. Proc Natl Acad Sci USA 1995; 92:7162-7166.
Bonfoco E, Leist M, Zhivotovsky B, Orrenius S, Lipton S A, Nicotera P. Cytoskeletal breakdown and apoptosis elicited by NO-donors in cerebellar granule cells require NMDA-receptor activation. J Neurochem 1996; 67:2484-2493.
Bonfoco E, Zhivotovsky B, Rossi A, Agullar-Santelises A, Orrenius S, Lipton SA, Nicotera P. BCL-2 delays apoptosis and PARP cleavage induced by NO-donors in GT1-7 cells. NeuroReport 1996; 8:273-276.
Bossy-Wetzel E, Lipton S A. Nitric Oxide signaling regulates mitochondrial number and function. Cell Death Diff, in press.
Bossy-Wetzel E, Talantova M V, Digicaylioglu M, Ju C, Zhang D, Lipton S A. Apoptotic cell shrinkage: regulation of voltage-gated potassium channels in NO induced neurotoxicity [Abstract]. Soc Neurosci Abstr 2001; 27:1788.
Bossy-Wetzel, E, Talantova M V, Lee W D, Schoelzke M, Goetz T, Mathews E, Harrop A, Lipton S A. Molecular mechanism of apoptotic cell shrinkage during neurotoxicity induced by nitric oxide [Abstract]. Soc Neurosci Abstr 2002; 225.5.
Brown S, Bernardo M M, Li Z H, Kotra L P, Tanaka Y, Fridman R, Mobashery S (2000) Potent and selective mechanism-based inhibition of gelatinases. J Am Chem Soc 122:6799-6800.
Budd S, Lipton S A. Calcium tsunamis: do astrocytic signaling waves transmit cell death messages via gap junctions during ischemia Nature Neurosci 1998; 1:431-432.
Budd S, Lipton S A. Mitochondrial-signaling for apoptosis following NMDA receptor activation is related to an energy crisis [Abstract], Soc Neurosci Abstr 199925:2131.
Budd S L, Kohli S S, Lipton S A. Apoptosis in cerebrocortical neurons is initiated by more than mitochondrial dysfunction [Abstract[. Soc Neurosci Abstr 2000; 26:1011.
Budd S L, Lee, W, Lipton S A. Mitochondria—energetic co-ordinators of apoptosis [Abstract]. Eur Soc Neurochem, Advances in Molecular Mechanisms of Neurological Disorders, Perugia, Italy, May 22-25, 2001.
Budd S L, Lipton S A. Excitotoxic NMDA receptor stimulation initiates a cytochrome c-mediated apopotitc pathway. Symposium on Neuronal Apoptosis, Universitätsklinikum Tübingen, August, 1999, p. 33.
Budd S L, Lipton S A. Mitochondrial permeability transition and membrane potential in NMDA-induced apoptosis [Abstract]. Soc Neurosci Abstr 1998; 24:1453.
Budd S L, Lipton S A. Signalling events in NMDA receptor-induced apoptosis in cerebrocortical cultures. NY Acad Sci 1999; 893:261-264.
Budd S L, Tenneti L, Lishnak T, Lipton S A. Mitochondrial and extramitochondrial apoptotic signaling pathways in cerebrocortical neurons. Proc Natl Acad Sci USA 2000; 97:6161-6166.
Bukrinsky M I, Nottet H S L M, Schmidtmayerova H, Dubrovsky L, Flanagan C R, Mullins M E, Lipton S A, Gendelman, H E. Regulation of nitric oxide synthase activity in human immunodeficiency virus type 1 (HIV-1)-infected monocytes: Implications for HIV-associated neurological disease. J Exp Med 1995; 181:735-745.
Cardone, M. H., Salvesen, G. S., Widmann, C, Johnson, G., and Frisch, S. M., The regulation of anoikis: MEKK-1 activation requires cleavage by caspases, Cell, 90, 315-323 (1997).
Castellanos M, Leira R, Serena J, Pumar J M, Lizasoain I, Castillo J, Davalos A (2003) Plasma metalloproteinase-9 concentration predicts hemorrhagic transformation in acute ischemic stroke. Stroke 34:40-46.
Chatterton J E, Awobuluyi M, Cui J, Sevarino K A, Lipton S A, Zhang D. Cloning and characterization of a new member of the NMDA receptor gene family, NMDAR3B [Abstract]. Soc Neurosci Abstr 2001; 27:1849.
Chatterton J E, Awobuluyi M, Premkumar L S, Takahashi H, Talantova M, Shin Y, Cui J, Sevarino K A, Tu S, Nakanishi N, Tong G, Lipton S A, Zhang D. Excitatory glycine receptor containing the NR3 family of NMDA receptor subunits. Nature 2002; 415:793-798.
Chen H-S V, Choi Y-B, Lipton S A. Poly-5-nitrosylation of cysteine residues mediates inhibition of NMDAR activity by nitric oxide-related species [Abstract]. Soc Neurosci Abstr 1999; 25:24.
Chen H-S V, Wang Y F, Rayudu P Y, Edgecomb P, Neill J C, Segal M M, Lipton S A, Jensen F E. Neuroprotective concentrations of the NMDA open-channel blocker memantine are effective without cytoplasmic vacuolization following post-ischemic administration and do not block maze learning or LTP. Neuroscience 1998; 86:1121-1132.
Chen Z L, Indyk J A, Strickland S (2003) The hippocampal laminin matrix is dynamic and critical for neuronal survival. Mol Biol Cell 14:2665-2676.
Chen Z L, Strickland S (1997) Neuronal death in the hippocampus is promoted by plasmin-catalyzed degradation of laminin. Cell 91:917-925.
Cheung W, Bhan I, Lipton S A. Nitric oxide (NO.) destabilizes filopodia while nitrosonium donors (NO+) induce outgrowth by rat retinal ganglion cells in vitro [Abstract]. Soc Neurosci Abstr 1996; 22:734.
Cheung W S, Bhan I, Lipton S A. Nitric oxide (NO.) stabilizes whereas nitrosonium (NO+) enhances filopodial outgrowth by rat retinal ganglion cells in vitro. Brain Res 2000; 868:1-13.
Cheung W S, Chen H-S V, Zhang X, Lipton S A. S-Nitrosylation of putative critical regulatory sulfhydryl groups in recombinant cyclic nucleotide-gated channels [Abstract]. Soc Neurosci Abstr 1997; 23:1479.
Choi Y-B, Chen H-S V, Lipton S A. Multiple cysteine residues on different subunits are involved in redox modulation of NR1/NR2A receptors [Abstract]. Soc Neurosci Abstr 1997; 23:932.
Choi Y-B, Chen H-S V, Lipton S A. Three pairs of cysteine residues mediate both redox and Zn²⁺ modulation of the NMDA receptor. J Neurosci 2001; 21 392-400.
Choi Y-B, Chen H-S V, Lipton S A. Three pairs of redox-sensitive cysteine residues are involved in high-affinity inhibition of NMDAR1/NMDAR2A receptors by Zn2+ [Abstract]. Soc Neurosci Abstr 1998; 24:342.
Choi Y-B, Chen H-S V, Sucher N J, Lipton S A. Redox modulatory site(s) of recombinant NMDA receptors: subunit composition and conformation [Abstract]. Soc Neurosci Abstr 1996; 22:1761.
Choi Y-B, Lipton S A. Redox modulation of the NMDA receptor. In: NMDA Receptors, Shaw C, Pasqualotto B, eds. Methods in Enzymology: Cellular and Molecular Life Sciences, 2000; 57:1535-1541.
Choi Y-B, Tenneti L, Le D A, Ortiz J, Bai G, Chen H-S V, Lipton S A. Molecular basis of NMDA receptor-coupled ion channel modulation by S-nitrosylation. Nature Neuroscience 2000; 3:15-21.
Chuang K I, Digicaylioglu M, Ju C, Lipton S A, Lyden P D. VEGF neuroprotection against NMDA insult in primary cerebrocortical cultures involves the VEGFR2-PI3kinase-phospho-AKT pathway [Abstract]. Soc Neurosci Abstr 2001; 27:2028.
Coussens L M, Fingleton B, Matrisian L M (2002) Matrix metalloproteinase inhibitors and cancer; trials and tribulations. Science 295:2387-2392.
Das S, Sasaki Y F, Rothe T, Premkumar L S, Takasu M, Crandall J E, Dikkes P, Connor D A, Rayudu P V, Cheung W, Chen H-S V, Lipton S A Nakanishi N. Increased NMDA current and spine density in mice lacking the NMDA receptor subunit NR3A Nature 1998; 393:377-381.
Dawson, T. M., and Snyder, S. H., Gases as biological messengers: nitric oxide and carbon monoxide in the brain, Journal of Neuroscience, 14, 5147-5159 (1994).
D'Emilia D, Sucher N J, Stamler J S, Lipton S A. Balance between thiol and S-nitrosothiol determines neurotoxicity in cortical cultures [Abstract]. Soc Neurosci Abstr 1996; 22:1920.
D'Emilia D M, Garden G A, Lipton S A. HIV-gp120 induced neuronal apoptosis is mediated by caspase enzymes [Abstract]. Soc Neurosci Abstr 1998; 24:1447.
D'Emilia D M, Lipton S A. Ratio of S-nitrosohomocyst(e)ine to homocysteine or other thiols determines neurotoxicity in rat cerebrocortical cultures, Neurosci Lett 1999; 265:103-106.
Digicaylioglu H M, Lipton S A. Erythropoietin protects rat central neurons from NMDA- and NO-induced apoptosis (Abstract). Soc Neurosci Abstr 1997, 23:1667.
Digicaylioglu M, Lipton S A, Erythropoietin (EPO) protects against nitric oxide-induced neuronal apoptosis via a Jak-2/NF-B signaling pathway (Abstract). Soc Neurosci Abstr 1999; 25:1088.
Digicaylioglu M, Lipton S A. Erythropoietin mediated neuroprotection involves cross-talk between Jak2 and NF-B signalling cascades. Nature 2001; 412:641-647.
Digicaylioglu M, Moayeri N, Lipton S A. Neuroprotection from nitric oxide by erythropoietin (EPO) is mediated by NF B (Abstract). 1998; 24:1792.
Digicaylioglu M. H., Lipton S. A. Erythropoietin-induced neuroprotection is mediated by crosstalk between the Janus kinase-2 and Nuclear Factor-B signaling pathways (Abstract). Soc Neurosci Abstr 2000; 26:774,
Dreyer E B, Lipton S A. New perspectives on glaucoma. JAMA 1999; 281:306-308,
Dreyer E B, Zurakowski D, Gorla M, Vorwerk C K, Lipton S A. The contribution of various nitric oxide synthase gene products to HIV-1 coat protein (gp120)-mediated retinal ganglion cell injury. Invest Opthalmol V is Sci 1999; 40:983-989.
Epstein L G, Gendelman H E, Lipton S A. Human immunodeficiency virus-1 neuropathogenesis. In: Berger J R, Levy R M, eds. AIDS and the Nervous System, 2nd ed. Philadelphia; Lippincott-Raven, 1996, pp. 59-75.
Farkas R H, Lipton S A. Pressure increases NMDA receptor activity of retinal ganglion cells: possible role in glaucoma (Abstract). Invest Opthalmol Vis Sci 2000; 4I:S899.
Felix A C, Tracey T, Kleefield J, Lipton S, Gonzalez R G, Navia B. HIV leucoencephalopathy—a unique disorder of AIDS: clinical and pathogenetic features (Abstract). Neurology 1998; 50:A217.
Frisch S M, Francis H (1994) Disruption of epithelial cell-matrix interactions induces apoptosis. J Cell Biol 124:619-626.
Garden G, Budd S, Tsai E, Singh S, Kaul M, Masliah E, Lipton S. Caspase enzymes in HIV induced neuronal injury and apoptosis [Abstract]. 4th International Symposium on Neurovirology. 10th Conference on Neuroscience of HIV Infection. 18-22 Jun. 2002, Düsseldorf, Germany (Abstract). J Neurovirol. 2002; 8(Suppl 1):42.
Garden G, Budd S L. Tsai E, Hanson L, Kaul M, D'Emilia D M, Friedlander R M, Yuan J, Masliah E, Lipton S A. Caspase cascades in HIV-associated neurodegeneration. J Neurosci 2002; 22:4015-4024.
Garden G, Tsai E, Budd S, Lishnak T, Lipton S. Caspase-3-like activity in HIV/gp120-induced apoptosis [Abstract]. Soc Neurosci Abstr 1999; 25:281.
Garden G A, Budd S L, Kaul M, Lipton S A. HIV-gp120 induces apoptosis in cultured cortical neurons by pathways involving caspases 8 and 9 and TNF-(Abstract). Soc Neurosci Abstr 2000; 26:1062.
Garden G A, Budd S L, Lipton S A. Is caspase enzyme inhibition a potential treatment modality for HIV demenita [Abstract]? Neurology 2000; (Suppl 3), A252.
Garden G A, Tsai E, Hanson L, Singh S, Torelli R, Tun C, Guo W, Lipton S A. Caspase inhibition prevents CNS injury in the HIV/gp120 transgenic mouse [Abstract]. Soc Neurosci Abstr 2001; 27:1746.
Gasche, Y., Fujimura, M., Morita-Fujimura, Y., Copin, J. C, Kawase, M., Massengale, J., and Chan, P. H., Early appearance of activated matrix metalloproteinase-9 after focal cerebral ischemia in mice: a possible role in blood-brain barrier dysfunction, Journal of Cerebral Blood Flow & Metabolism, 19, 1020-1028 (1999).
Gendelman H E, Eiden L, Epstein L G, Grant I, Lipton S A, McArthur J, Pomerantz R, Price R, Swindells S. The Neuropathogenesis of HIV-1-dementia. In: The Neurology of AIDS, Gendelman H E, Lipton S A, Epstein L G, Swindells S, eds. New York: Chapman and Hall, 1998, pp. 1-10.
Giannelli G, Falk-Marzillier J, Schiraldi O, Stetler-Stevenson W G, Quaranta V (1997) Induction of cell migration by matrix metalloprotease-2 cleavage of laminin-5. Science 277:225-228.
Gillessen T, Budd S L. Lipton S A. Excitatory amino acid neurotoxicity. In: Molecular and Cellular Biology of Neuroprotection in the CNS, Alzheimer C, ed., Eureka Press, in press.
Gu Z, Kaul M, Kridel S J, Ju C, Smith J W, Lipton S A. Induction of neuronal apoptosis by matrix metalloproteinase-9 (MMP-9) in rat cerebrocortical cultures [Abstract]. Soc Neurosci Abstr 2001; 27:299.
Gu Z, Kaul M, Yan B, Kridel S J, Cui J, Strongin A, Smith J W, Liddington R C, Lipton S A. S-Nitrosylation of matrix metalloproteinases: signaling pathway to neuronal cell death. Science 2002; 297:1186-1190.
Gu Z, Kaul M, Yan B, Kridel S J, Cui J, Strongin A, Smith J W, Liddington R C, Lipton S A. Activation of matrix metalloproteinase-9 triggered by nitric oxide (NO): An extracellular signaling pathway to neuronal cell death [Abstract]. Soc Neurosci Abstr 2002; 493.13.
Heo J H, Lucero J, Abumiya T, Koziol J A, Copeland B R, del Zoppo G J (1999) Matrix metalloproteinases increase very early during experimental focal cerebral ischemia. J Cereb Blood Flow Metab 19:624-633.
Horstmann S, Kalb P, Koziol J, Gardner H, Wagner S (2003) Profiles of matrix metalloproteinases, their inhibitors, and laminin in stroke patients: influence of different therapies. Stroke 34:2165-2170.
Huang, Z., Huang, P. L., Panahian, N., Dalkara, T., Fishman, M. C, and Moskowitz, M. A., Effects of cerebral ischemia in mice deficient in neuronal nitric oxide synthase, Science, 265, 1883-1885 (1994).
Indyk J A, Chen Z L, Tsirka S E, Strickland S (2003) Laminin chain expression suggests that laminin-10 is a major isoform in the mouse hippocampus and is degraded by the tissue plasminogen activator/plasmin protease cascade during excitotoxic injury. Neuroscience 116:359-371.
Itano H, Okamoto S, Zhang D, Lipton S A, Ruoslahti E. Cell spreading controls endoplasmic and nuclear calcium: a physical gene regulation pathway from the cell surface to the nucleus. Proc Natl Acad Sci USA, in press.
Jaffrey, S. R, Erdjument-Bromage, H., Ferris, C, D., Tempst, P., and Snyder, S. H., Protein S-nitrosylation: a physiological signal for neuronal nitric oxide, Nature Cell Biology, 3, 193-197 (2001).
Jia, L., Bonaventura, C, Bonaventura, J., and Stamler, J. S., S-nitrosohaemoglobin: a dynamic activity of blood involved in vascular control, Nature, 380, 221-226 (1996).
Johnston, J. B Zhang, K.; Silva, C, Shalinsky, D, R., Conant, K., Ni, W., Corbett, D., Yong, V. W., and Power, C, HIV-1 Tat neurotoxicity is prevented by matrix metalloproteinase inhibitors, Annals of Neurology, 49, 230-241 (2001).
Kaul M, Garden G A, Lipton S A. Pathways to neuronal injury and apoptosis in HIV-associated dementia. Nature 2001; 410:988-994.
Kaul M, Lipton S A. Chemokines and activated macrophages in gp120-induced neuronal apoptosis. Proc Natl Acad Sci USA 1999; 96:8212-8216.

Kaul M, Lipton S A. HIV Infection and the Central Nervous System. In: Neuroimmune Endocrinology. Greenen V, ed., in press.
Kaul M, Lipton S A. Knock out of HIV-1 coreceptors attenuates neuronal apoptosis induced by HIV envelope glycoprotein GP120 [Abstract], Soc Neurosci Abstr 2001; 27:1789.
Kaul M, Lipton S A. Neuronal apoptosis induced by HIV-1 gp120 and -chemokine SDF-1 is attenuated by β-chemokines and p38 MAP kinase inhibitors [Abstract]. Neurology 2000; (Suppl 3), A252-A253.
Kaul M, Lipton S A. Neuronal apoptosis induced by HIV-gp120 and the chemokines SDF-1 and involves signaling by p38 MAP kinase [Abstract], Soc Neurosci Abstr 1999; 25:1304.
Kaul M, Lipton S A. NMDA receptors mediate AIDS-related damage in the central nervous system. In: Therapeutic Potential of Ionotropic Glutamate Receptor Antagonists and Modulators. Danysz W, Parsons C, eds., in press.
Kaul M, Lipton S A. The NMDA receptor—its role in neuronal apoptosis and HIV-associated dementia. Science Online: NeuroAIDS 2000; 3:1-5.
Kaul M, Lipton S A. The role of the NMDA receptor in neuronal apoptosis and HIV-associated dementia. In: Glutamate & Addiction, Herman B H, Frankenheim J, Litten R, Sheridan P, Weight F F, Zukin S, eds. Totowa, N.J.: Humana, 2001.
Kaul M, Lipton S A. β-Chemokines or knockout of CXCR4 chemokine receptor attenuates neuronal apoptosis induced by HIV-1 gp120 and SDF-1 [Abstract]. Soc Neurosci Abstr 2000; 26:1062,
Kaul M, Medders K E, Lipton S A. HIV-1 envelope glycoprotein gp120 deregulates activity of stress-activated protein kinases and can induce neuronal death in the absence of HIV coreceptors CXCR4 and CCR5 [Abstract]. Soc Neurosci Abstr 2002; 93.16.
Kikuchi M, Bai G, Vorwerk C K, Dreyer E, Lipton S A. Signaling by p38 mitogen-activated protein kinase (MAPK) in axotomy-induced apoptosis of rat retinal ganglion cells [Abstract]. Soc Neurosci Abstr 1999; 25:757.
Kikuchi M, Tenneti L, Lipton S A. Role of p38 mitogen-activated protein kinase in axotomy-induced apoptosis of rat retinal ganglion cells, J Neurosci 2000; 20:5037-5044.
Kikuchi M, Tenneti L, Okamoto S-I, Lipton S A. p38 Mitogen activated-protein kinase mediates axotomy-induced apoptosis of rat retinal ganglion cells [Abstract]. Soc Neurosci Abstr 2000; 26:109.
Kim W-K, Choi Y-B, Rayudu P V, Das P, Asaad W, Arnelle D R, Stamler J S, Lipton S A. Attenuation of NMDA receptor activity and neurotoxicity by nitroxyl (NO—). Neuron 1999; 24:461-469.
Kim W-K, Rayudu P V, Mullins M E, Stamler J S, Lipton S A. Down regulation of NMDA receptor activity in cortical neurons by peroxynitrite. In: The Biology of Nitric Oxide; part 5, Stamler J S, Gross S, Moncada S, eds, London: Portland Press, 1996, p, 26.
Kim W-K, Stamler J S, Lipton S A. Redox congeners of nitric oxide, the NMDA receptor, and intracellular Ca2+. In: Nitric Oxide, a vol. of Methods in Neurosciences, Maines M D, ed., Orlando: Academic Press, 1996.
Kleifeld, O., Kotra, L. P., Gervasi, D. C., Brown, S., Bernardo, M. M., Fridman, R., Mobashery, S., and Sagi I., X-ray absorption studies of human matrix metalloproteinase-2 (MMP-2) bound to a highly selective mechanism-based inhibitor, comparison with the latent and active forms of the enzyme, Journal of Biological Chemistry, 276, 17125-17131 (2001).
Kridel, S. L, Chen, E., Kotra, L. P., Howard, E. W., Mobashery, S., and Smith, J. W., Substrate hydrolysis by matrix metalloproteinase-9, Journal of Biological Chemistry, 276, 20572-20578 (2001).
Kumura, E., Kosaka, H., Shiga, T., Yoshimine, T., and Hayakawa, T., Elevation of plasma nitric oxide end products during focal cerebral ischemia and reperfusion in the rat, Journal of Cerebral Blood Flow & Metabolism, 14, 487-491 (1994).
Lapchak P A, Chapman D F, Zivin J A (2000) Metalloproteinase inhibition reduces thrombolytic (tissue plasminogen activator)-induced hemorrhage after thromboembolic stroke. Stroke 31:3034-3040.
Le D, Das S, Wang Y F, Yoshizawa T, Sasaki Y F, Takasu M, Nemes A, Mendelsohn M, Dikkes P, Lipton S A, Nakanishi N. Enhanced neuronal death from focal ischemia in AMPA-receptor transgenic mice. Molec Brain Res 1997; 52:235-241.
Le D, Lipton S A. Potential and current use of N-methyl-D-aspartate (NMDA) receptor antagonists in diseases of aging. Drugs & Aging 2001; 18:717-724.
Le D A, Wang Y, Kaul M, Lipton S A. Neuroprotection by salicylhydroxamic acid (SHA) is in part mediated by scavenging of free radicals [Abstract]. Soc Neurosci Abstr 1999; 25:299.
Lee S R, Tsuji K, Lo E H (2004) Role of matrix metalloproteinases in delayed neuronal damage after transient global cerebral ischemia. J Neurosci 24:671-678.
Lee W D, Lipton S A, Budd S L. Mitochondria maintain respiratory capacity after release of cytochrome C (Abstract). Soc Neurosci Abstr 2002; 892.15.
Li M, Ona V O, Chen M, Kaul M, Tenneti L, Stieg, P E, Lipton S A, Friedlander R M. Functional role and therapeutic implications of neuronal caspase-1 and -3 in a mouse model of traumatic spinal cord injury. Neuroscience 2000; 99:333-342.
Lipton S A Bossy-Wetzel E. Dueling activities of AIF in cell death versus survival: DNA binding and redox activity. Cell 2002; 111:147-150.
Lipton S A, Choi Y-B, Chen H-S V, Godzik A, Bankston L. Neurotoxicity of NMDA: Novel therapeutic approaches via nitric oxide-mediated modulation (Abstract). Oxygen Club of California 2002 World Congress, Santa Barbara, Calif. Oxidants and Antioxidants in Biology, Mar. 6-9, 2002, pp. 82-83.
Lipton S A, Choi Y-B, Godzik A, Bankston L. Nitric oxide-mediated modulation of NMDA receptor activity (Abstract). Nitric oxide, cell survival and cell death. 1st Wolfson Institute International Conference, 14-15 Sep. 2001, University College London, p. 10.
Lipton S A, Choi Y-B, Sucher N J, Chen H-S V. Neuronal protection by nitric oxide-related species. In: Nitric Oxide in the Eye, S. Kashii, H. Honda and A. Akaike, eds, Springer-Verlag, Tokyo, May 2000.
Lipton S A, Choi Y-B, Sucher N J, Chen H S-V. Neuroprotective vs. neurodestructive effects of NO-related species. Proceedings of the Intn'tl Free Radical Society, Barcelona. BioFactors 1998; 8:33-40.
Lipton S A, Choi Y-B, Sucher N J, Chen H S-V. NO-NMDA receptor interactions: A neuromolecular approach to novel therapeutics. Proceedings of the Ernst Schering A G Research Foundation, Berlin, May 1997.
Lipton S A, Choi Y-B, Sucher N J, Pan Z-H, Stamler J S. Redox state, NMDA receptors, and NO-related species, Trends Pharmacol Sci 1996; 17:186-187.
Lipton S A, Choi Y-B, Takahashi T, Zhang D, Li W, Godzik A, Bankston L A. Cysteine regulation of protein function—as exemplified by NMDA-receptor modulation. Trends Neurosci. 2002; 25:474-480.
Lipton S A, Choi Y-B, Tenneti L, D'Emilia D M, Chen H-S V. S-Nitrosylation of NMDA receptor and caspases affords neuroprotection from NMDA receptor-mediated insults. In: Pharmacology of Cerebral Ischemia, 1998. Krieglstein J, ed. Medpharm Scientific Publishers, Stuttgart, pp. 217-226,
Lipton S A, Garthwaite, L Neuronal systems for the analysis of the effects of NO and redox-related congeners. In: Feelisch M, Stamler J S, eds. Methods in Nitric Oxide Research. London: John Wiley and Sons, 1996, pp. 593-602.

Lipton S A, Gelbard H A. Development of adjunctive therapies for the neurological manifestations of AIDS: dementia and painful neuropathy. In: The Neurology of AIDS, 2nd Ed., Gendelman H E, Lipton S A, Grant I, Everall I, Swindells S, eds, New York; Oxford University Press, in press.
Lipton S A, Gendelman H E. Dementia associated with the acquired immunodeficiency syndrome. N Engl J Med 1995; 332:934-940.
Lipton S A, Gu Z, Yan B, Kridel S J, Cui J K, Strongin A, Smith J W, Liddington R C, Kaul M. S-Nitrosylation and subsequent sulfonation of matrix metalloproteinases produce an extracellular signaling pathway for neuronal cell death. In: Pharmacology of Cerebral Ischemia, 2002. Krieglstein J, Klumpp S, eds. Medpharm Scientific Publishers, Stuttgart, pp. 233-241.
Lipton S A, Heniemann, S F. Molecular basis for the potential use of NMDA receptor Open-channel blockers in the treatment of cerebral ischemia and other brain insults. In: Cardiovascular Diseases, Chapter 28, Stroke: Molecular Insights. Chien K, editor, in press.
Lipton S A, Kaul M, Glutamate- and chemokine-receptors in neuronal vulnerability. Proceedings of the NIH/NINDS Neurology of AIDS Program Panel 2, Miami, Fla., February 1999. J Neurovirology.
Lipton S A, Kaul M. Role of chemokines and activated macrophages in HIV gp120-induced neuronal apoptosis [Abstract]. Keystone Meeting on Effectors of Inflammation in the CNS, Taos, N. Mex., Mar. 9-14, 1999, p. 50.
Lipton S A, Kieburtz K. Development of adjunctive therapies for the neurologic manifestations of AIDS: Dementia and painful neuropathy. In: The Neurology of AIDS, Gendelman H E, Lipton S A, Epstein L G, Swindells S, eds. New York: Chapman and Hall, 1998, pp. 377-381.
Lipton S A, Kim W-K, Choi Y-B, Kumar S, D'Emilia D M, Rayudu P V, Arnelle D R, Stamler J S. Dual actions of homocysteine at the NMDA receptor. Proc Natl Acad Sci USA 1997; 94:5923-5928.
Lipton S A, Kim W-K, Rayudu P V, Asaad W, Arnelle D R, Stamler J S. Nitroxyl anion (NO−) decreases NMDA receptor activity and neurotoxicity (Abstract). Neurology 1997; 48:A81-A82.
Lipton S A, Kim W-K, Rayudu P V, Asaad W, Amelle D R, Stamler J S. Singlet and triplet nitroxyl anion (NO−) lead to N-memyl-D-aspartate (NMDA) receptor downregulation and neuroprotection. In: The Biology of Nitric Oxide, part 5, Stamler J S, Gross S, Moncada S, eds, London: Portland Press, 1996, p. 125.
Lipton S A, Kim W-K, Stamler J S. Neuroprotective and neuropathotogic effects of nitric oxide—Role of the NMDA receptor. In: Free Radicals in Brain Physiology and Disorders, Packer L, Hiramatsu M, Yoshikawa T, eds, Orlando: Academic Press, 1996, pp. 71-82.
Lipton S A, Nakanishi N. Shakespeare in love—with NMDA receptors? Nature Medicine 1999; 5:270-271. (News and Views article).
Lipton S A, Nicotera P. Calcium, free radicals and excitotoxins in neuronal apoptosis. Cell Calcium 1998; 23:165-171.
Lipton S A, Nicotera P. Excitotoxicity, free radicals, necrosis, and apoptosis. The Neuroscientist 1998; 4:345-352.
Lipton S A, Rayudu, P V, Choi Y-B, Sucher N J, Chen H S V. Redox modulation of the NMDA receptor by NO-related species. Progress in Brain Research, R R Mize, V Dawson, T M Dawson, M Friedlander, eds., Elsevier, Amsterdam, 1998; 118:73-82.
Lipton S A, Stamler J S. NO—friend or foe. In Neuroprotection: Fundamental and Clinical Aspects. Bär P R, Beal M F, eds, Marcel Dekker, Inc., New York, 1998.
Lipton S A, Tenneti L, Budd, S L. Caspases, mitochondrial depolarization, and permeability transition in NMDA-induced apoptosis [Abstract]. J Neurochem 1999; 72:S90.
Lipton S A, Wang Y F. NO-related species can protect from focal cerebral ischemia/reperfusion. In: Pharmacology of Cerebral Ischemia, 1996. Krieglstein J, Oberpichler-Schwenk H, eds. Wissenschaftliche Verlagsgesellschaft mbH, Stuttgart, 1996. pp. 183-491.
Lipton S A, Wong H-k, Chan S F, Premkumar L S, Rothe T, Chen H-S V, Zhang D, Trimmer J S, Das S, Nakanishi N, Sucher N J. NR3A: Single channel currents, electrophysiology of knock-out mice, and characterization with a monoclonal antibody (Abstract), Soc Neurosci Abstr 1998; 24:1271.
Lipton S A. AIDS Dementia and Stroke: Potential Treatment with NMDA Open-Channel Blockers and Nitric Oxide-Related Species. In: Delaying Dysfunction and Death in Neurones and Disintegration of Synapses—Prospects for Developing Treatment Strategies. Barnes J, Price D L, eds. Henry Stewart Conference Studies, 1996.
Lipton S A. AIDS dementia as a form of excitotoxicity: potential therapy with NMDA open-channel blockers and redox congeners of nitric oxide. In: Neurodegenerative diseases: molecular and cellular mechanisms and therapeutic advances, Fiskum G, ed. Plenum Publishing Corp., 1996.
Lipton S A. Apoptotic and necrotic excitotoxicity in AIDS dementia and stroke: Potential treatment with NMDA open-channel blockers and NO. In: Proceedings of the Korean Neuroscience Society Plenary Lectures, Seoul, June 1997.
Lipton S A. Apoptotic and necrotic excitotoxicity in AIDS dementia and stroke: potential treatment [Abstract]. Korean Neuroscience Association Meeting, Ewha Medical Center, Seoul, Korea, Jun. 27, 1997.
Lipton S A. Clinically-tolerated NMDA receptor antagonists and newly cloned NMDA receptor subunits that mimic them. In: Proceedings of the 22nd Princeton Conference on Cerebrovascular Disease. Chan P H, editor. Cambridge Univ. Press, 2001.
Lipton S A. Distinctive chemistries of NO-related species. Neurochem Int 1996; 29:111-114.
Lipton S A. Excitotoxic apoptosis and necrosis in AIDS dementia and stroke: potential treatment with NMDA open-channel blockers and NO-related species (Abstract). Japan Intractable Diseases Research Foundation, Development of Brain Science and Diseases, International Seminar on Neurobiology X, Tokyo, Japan, Jan. 20-21, 1998, p. 22.
Lipton S A. Excitotoxins, free radicals, and apoptosis in AIDS dementia and focal cerebral ischemia: treatment with NMDA open-channel blockers and nitric oxide-related species (Abstract). Brain Res Assoc, 14th Annual Meeting, Liverpool, UK 1997; 14:84,
Lipton S A. HIV-related neuronal injury; potential therapy with NMDA open-channel blockers and redox congeners of nitric oxide. In: Excitatory Amino Acids and the Cerebral Cortex, Conti F, Hicks T P, eds. MIT Press/Bradford Books, Cambridge, Mass., 1996, pp. 253-260,
Lipton S A. Janus Faces of NF-B: Neurodestruction versus neuroprotection. Nature Medicine 1997; 3:20-22.
Lipton S A. Mechanisms of neurological injury. In: Brain Injury and Pediatric Cardiac Surgery, Jonas R A, Newburger J W, Volpe J J, eds, Boston: Butterworth-Heinemann Publishers, 1996, pp. 229-238.
Lipton S A. NeuroAIDS: The etiology and treatment of HIV-associated dementia. In: Int'l Workshop on Neuronal Injury in MS and Related Disorders: Mechanisms and Prevention, New Orleans, La. Mar. 18-21, 2001, pp. 13-14.
Lipton S A. Neuronal injury associated with HIV-1: Approaches to treatment. Ann Rev of Pharmacol Toxicol 1998; 38:159-177.
Lipton S A. Neuronal injury in AIDS dementia: Potential treatment with NMDA open-channel blockers and nitric oxide-related species. Korean International Meeting of Pharmacology, 1996.
Lipton S A. Neuronal protection and destruction by NO. Cell Death Diff 1999; 6:943-951.
Lipton S A. Neuropathogenesis of acquired immunodeficiency dementia. Curr Neurol 1997; 10:247-253.
Lipton S A. Neuroprotective and neurodestractive mechanisms of NO-related species [Abstract]. Harvard Medical International-ASAN Medical Center Conference on Cerebrovascular Disease, Seoul, Korea, Jul. 4-5, 1997.
Lipton S A. Nitric oxide and related molecules in neuronal death and survival. The Neurologist 1996.
Lipton S A. Nitric oxide and respiration. Nature 2001; 413:118-121.
Lipton S A. NMDA receptors and focal cerebral ischemia: novel mechanisms of modulation from work on recombinant receptors compared to native receptors [Abstract], Harvard Medical International-ASAN Medical Center Conference on Cerebrovascular Disease, Seoul, Korea, Jul. 4-5, 1997.
Lipton S A. NO in AIDS-associated neurologic disease. In: Nitric Oxide and Infection, Fang F C, ed. New York: Plenum, 1999.
Lipton S A. NO-related species: Neuroprotection versus neurodestruction. In: Primer on Cerebrovascular Diseases, Welch K M A, Caplan L, Reis D, Weir B, Siesjö B, eds., Orlando: Academic Press, 1997, pp. 210-214.
Lipton S A. Pathogenesis of AIDS dementia: potential treatment with NMDA open-channel blockers and NO-related species (Abstract). Keystone Symposia, C2: Molecular aspects of Viral Immunity. Tamarron, Colo., Feb. 16-22,1998, p. 30.
Lipton S A Redox modulation of the NMDA receptor by nitric oxide and related species [Abstract]. EAAS 97, ISN/ASN Satellite Symposium and ASPET Colloquium on Excitatory Amino Acids, Waterville Valley, N.H., Jul. 16-20,1997, p. 34.
Lipton S A. Redox sensitivity of NMDA receptors. In: Methods in Molecular Biology: NMDA Receptor Protocols, Li M, ed. Totowa, N.J.: Humana, 1999; 128:121-130.
Lipton S A. Retinal ganglion cells, glaucoma and neuroprotection. In: Concepts and Challenges in Retinal Biology—A Tribute to John E. Dowling. Progr Brain Res 2001; 131:712-718.
Lipton S A. Role for memantine in protecting retinal ganglion cells from glaucomatous damage. Surv Opthalmol, in press.
Lipton S A. Role of nitric oxide in neuronal protection versus apoptosis. In: Nitric Oxide Biology and Pathbiology, Ignarro L, ed. San Diego: Academic Press, 2000.
Lipton S A. Signaling pathways to neuronal apoptosis in ischemia and dementia: glutamate receptors and beyond [Abstract]. VI Workshop on Apoptosis in Biology and Medicine: Role of proinflammatory and chemotactic cytokines in normal and pathological brain. Hotel Porto Pirgos, Parghelia (VV), Calabria, Italy, 25-29 May, 2002, pp. 21-24.
Lipton S A. Similarity of neuronal cell injury and death in AIDS dementia and focal cerebral ischemia: potential treatment with NMDA open-channel blockers and nitric oxide-related species. Brain Pathol 1996; 6:507-517.
Lipton S A. S-Nitrosylation of the NMDA receptor: a novel mechanism of redox modulation (Abstract). Conference on Neurological Pharmacologic Therapeutics, Seoul National University, Jul. 2, 1997.
Lipton S A. Treating AIDS dementia. Science 1997; 276:1629-1630.
Lipton S A. Workshop on HIV-associated neural injury and apoptosis: excitotoxins, chemokines, and caspases (Abstract). 4th International Symposium on Neurovirology. 10th Conference on Neuroscience of HIV Infection. 18-22 Jun. 2002, Düsseldorf, Germany. L Neurovirol. 2002; 8(Suppl 1):41.
Lipton, S. A. Differential actions on neurons of the various redox-related forms of nitric oxide (NO+, NO., NO−). Third Mainzer Forum in Medicinal Chemistry, Johannes Gutenberg-Urnversität, Mainz, Germany, 1996.
Lipton, S. A., Choi, Y. B., Pan, Z. H., Lei, S. Z., Chen, H. S., Sucher, N. J., Loscalzo, J., Singel, D. J., and Stamler, J. S., A redox-based mechanism for the neuroprotective and neurodestructive effects of nitric oxide and related nitroso-compounds, Nature, 364, 626-632 (1993).
Lukes, A., Mun-Bryce, S., Lukes, M., and Rosenberg, G. A., Extracellular matrix degradation by metalloproteinases and central nervous system diseases, Molecular Neurobiology, 19, 267-284 (1999).
Manabe S, Lipton S A. NMDA-induced divergent signals leading to proapoptotic and antiapoptotic pathways in the rat retina. Invest Opthalmol Vis Neurosci 2003; 44:385-392.
Manabe S, Lipton, S A. p38 mitogen-activated protein kinase is involved in NMDA-induced retinal ganglion cell death in vivo [Abstract]. Soc Neurosci Abstr 2001; 27:697.
Matthews, J. R., Botting, C. H., Panico, M., Morris, H. R., and Hay, R. T., Inhibition of NF-kappaB DNA binding by nitric oxide, Nucleic Acids Research, 24, 2236-2242 (1996).
Melino, G., Bernassola, F., Knight, R. A., Corasaniti, M. T., Nistico, G., and Finazzi-Agro, A., S-nitrosylation regulates apoptosis, Nature, 388, 432-433 (1997).
Montaner, J., Alvarez-Sabin, J., Molina, C., Angles, A., Abilleira, S., Arenillas, J., Gonzalez, M. A., and Monasterio, J., Matrix metalloproteinase expression after human cardioembolic stroke: temporal profile and relation to neurological impairment, Stroke, 32, 1759-1766 (2001).
Morgunova, E., Tuuttila, A., Bergmann, U., Isupov, M., Lindqvist, Y., Schneider, G., and Tryggvason, K., Structure of human pro-matrix metalloproteinase-2: activation mechanism revealed, Science, 284, 1667-1670 (1999).
Muffins M E, Sondheimer N J, Huang Z, Singel D J, Huang P L, Fishman M C, Jensen F E, Lipton S A, Stamler J S, Moskowitz, M A. Spin-trapping NO in nNOS-deficient mice: indications for stroke therapy, In: The Biology of Nitric Oxide, part 5, Stamler J S, Gross S, Moncada S, eds, London; Portland Press, 1996, p. 9.
Nakanishi N, Das S, Sasaki Y, Tao G L, Takasu M, Rothe T, Lipton S A. Role of NR3A in cortical development (Abstract). Soc Neurosci Abstr 1998; 24:1271.
National Institute of Neurological Disorders and Stroke rt-PA Stroke Study Group (1995) Tissue plasminogen activator for acute ischemic stroke. N Engl J Med 333:1581-1587.
Navia B A, Dafni R, Simpson D, Tucker T, Singer E, McArthur J C, Yiannoutsos C, Zaborski L, Lipton S A. A phase I/II trial of nimodipine for HIV-related neurological complications. Neurology 1998; 51:221-228.
Nicotera P, Ankarcrona M, Bonfoco E, Orrenius S, Lipton S A. Neuronal necrosis and apoptosis: two distinct events induced by exposure to glutamate or oxidative stress. In: Neuronal Regeneration, Reorganization and Repair, Advances in Neurology series, Vol, 72, Seil F J, ed, Philadelphia: Lippincott-Raven Publishers, 1997, pp. 95-101.
Nicotera P, Ankarcrona M, Bonfoco E, Orrenius S, Lipton S A. Neuronal apoptosis versus necrosis induced by glutamate or free radicals. Apoptosis 1996; 1:5-10.
Nicotera P, Lipton S A. Excitotoxins and neuronal apoptosis versus necrosis. J Cereb Blood Flow Metab 1999; 19:583-591.
Okamoto S-i, Krainc D, Sherman K, Lipton S A. Antiapoptotic role of the p38 mitogen-activated protein kinase-myocyte enhancer factor 2 transcription factor pathway during neuronal differentiation. Proc Natl Acad Sci USA 2000; 97:7561-7566,
Okamoto S-i, Li Z, Ju C, Schölzke M N, Matthews E, Cui J, Salvesen G S, Bossy-Wetzel E, Lipton S A. Dominant-interfering forms of MEF2 generated by caspase cleavage contribute to NMDA-induced neuronal apoptosis. Proc Natl Acad. Sci. USA 2002; 99:3974-3979.
Okamoto S-I, Lipton S A. Antiapoptotic role of the p38 MAP kinase/MEF2 transcription factor pathway during neuronal differentiation of P19 cells (Abstract), Soc Neurosci Abstr 2000; 26:605.
Opdenakker G, Van den Steen P E, Van Damme J (2001) Gelatinase B: a tuner and amplifier of immune functions. Trends Immunol 22:571-579.
Overall C M, Lopez-Otin C (2002) Strategies for MMP inhibition in cancer: innovations for the post-trial era. Nat Rev Cancer 2:657-672.
Pan Z-H, Segal M M, Lipton S A. Nitric oxide-related species inhibit evoked neurotransmission but enhance spontaneous miniature synaptic currents in central neuronal cultures. Proc Natl Acad Sci USA 1996; 93:15234-15428.
Pan Z-H, Segal M M, Lipton S A. Redox congeners of nitric oxide produce presynaptic inhibition of evoked synaptic currents but enhancement of spontaneous miniature synaptic currents in hippocampal/cortical neurons. In: The Biology of Nitric Oxide, part 5, Stamler J S, Gross S, Moncada S, eds, London: Portland Press, 1996, p. 22.
Rayudu P V, D'Emilia D, Gaudiano P, Lipton S A. Apoptosis vs. necrosis: role of Ca2+ and energy failure (Abstract). Soc Neurosci Abstr 1997; 23:2291.
Romanic A M, White R F, Arleth A J, Ohlstein E H, Barone F C (1998) Matrix metalloproteinase expression increases after cerebral focal ischemia in rats: inhibition of matrix metalloproteinase-9 reduces infarct size. Stroke 29:1020-1030.
Rosenberg G A, Navratil M, Barone F, Feuerstein G (1996) Proteolytic cascade enzymes increase in focal cerebral ischemia in rat. J Cereb Blood Flow Metab 16:360-366.
Rothe T, Chen H-S V, Sucher N J, Das S, Nakanishi N, Lipton S A. Increased NMDA currents in cerebral cortex of NMDAR-L deficient mice [Abstract]. Soc Neurosci Abstr 1997; 23:948.
Sasaki Y F, Rothe T, Premkumar L S, Das S, Cui J: Wong H-k, Sucher N J, Zhang D, Nakanishi N, Lipton S A. Characterization and comparison of the NR3A subunit of the NMDA receptor in recombinant systems and primary cerebrocortical neurons. J Neurophysiol 2002; 87:2052-2063.
Sato, S., Tominaga, T., Ohnishi, T., and Ohnishi, S. T., Electron paramagnetic resonance study an nitric oxide production during brain focal ischemia and reperfusion in the rat, Brain Research, 647, 91-96 (1994).
Soriano S G, Coxon A, Wang Y F, Frosch M P, Lipton S A, Hickey P R, Mayadas T N. Mice deficient in Mac-1 (CD11b/CD18) are less susceptible to cerebral ischemia-reperfusion injury. Stroke 1999; 30:134-9,
Soriano S G, Lipton S A, Wang Y F, Springer T A, Gutierrez-Ramos J-C, Hickey P R. Intercellular adhesion molecule-1 (ICAM-1)-deficient mice are less susceptible to cerebral ischemia-reperfusion injury. Ann Neurol 1996; 39:618-624.
Soriano S G, Wang Y F, Lipton S A, Dikkes P, Gutierrez-Ramos J-C, Hickey P R. ICAM-1 is not required for neuronal apoptosis after transient focal cerebral ischemia. Brain Res 1998; 780:337-341
Stamler J S, Toone E J, Lipton S A, Sucher N J. (S)NO signals: translocation, regulation and a consensus motif. Neuron 1997; 18:691-696,
Stamler, J. S., and Hausladen, A., Oxidative modifications in nitrosative stress, Nature Structural Biology, 5, 247-249 (1998).
Stamler, J. S., Redox signaling: nitrosylation and related target interactions of nitric oxide, Cell, 78, 931-936 (1994).
Stieg P E, Le D A, Kaul M, Carroll M, Lipton S A. Cerebral infarcts in complement (C3)-deficient mice (Abstract). Soc Neurosci Abstr 1999; 25:317.
Stieg P E, Sathi S, Warach S, Le D A, Lipton S A., Neuroprotection by the NMDA open-channel blocker memantine in a photothrombotic model of cerebral focal ischemia in neonatal rat. Eur J Pharmacol 375:115-120, 1999.
Sucher N J, Lipton S A Dreyer E B. Molecular basis of glutamate toxicity in retinal ganglion cells. Vision Res 1997; 37:3483-3493.
Takasu M, Das S, Sasaki Y, Sucher N J, Lipton S A, Nakanishi N. Abnormal dendritic morphology in NMDA receptor-like (NMDAR-L) deficient mice [Abstract]. Soc Neurosci Abstr 1997; 23:947.
Tenneti L, D'Emilia D, Troy C M, Lipton S A, Role of caspases in NMDA-induced apoptosis of cerebrocortical neurons (Abstract). Soc Neurosci Abstr 199723:1667.
Tenneti L, D'Emilia D M, Lipton S A. Suppression of neuronal apoptosis by S-nitrosylation of caspases. Neurosci Lett 1997; 236:139-142.
Tenneti L, D'Emilia D M, Troy C M, Lipton S A. Role of caspases in N-methyl-D-aspartate-induced apoptosis in cerebrocortical neurons. J Neurochem 1998; 71:946-959.
Tenneti L, Lipton S A, Role of caspases in NMDA-induced apoptosis of cultured rat retinal ganglion cells (Abstract). Soc Neurosci Abstr 1999; 25:1604.
Tenneti L, Lipton S A. Activation of caspases during NMDA-induced apoptosis of cultured cerebrocortical neurons (Abstract). Soc Neurosci Abstr 1998; 24:1450.
Tenneti L, Lipton S A. Involvement of activated caspase-3-like proteases in N-methyl-D-aspartate-induced apoptosis in cerebrocortical neurons. J Neurochem 2000; 74:134-142.
Toggas, S. M., Masliah, E., Rockenstein, E. M., Rail, G. F., Abraham, C. R., and Mucke, L., Central nervous system damage produced by expression of the HIV-1 coat protein gp120 in transgenic mice, Nature, 367, 188-193 (1994).
Traynelis S F, Lipton S A. Is tissue plasminogen activator a threat to neurons Nature Med, 2001; 7:17-18.
Tsirka S E, Gualandris A, Amaral D G, Strickland S (1995) Excitotox in induced neuronal degeneration and seizure are mediated by tissue plasminogen activator. Nature 377:340-344.
Tu S, Funamoto C, Chatterton J E, Lipton S A, Nakanishi N. Generation of NR3A transgenic mice (Abstract). Soc Neurosci Abstr 2001; 27:1850.
Van Wart, H. E., and Birkedal-Hansen, H., The cysteine switch: a principle of regulation of metalloproteinase activity with potential applicability to the entire matrix metalloproteinase gene family, Proceedings of the National Academy of Sciences of the United States of America, 87, 5578-5582 (1990).
Wang X, Lee S R, Arai K, Tsuji K, Rebeck G W, Lo E H (2003) Lipoprotein receptor-mediated induction of matrix metalloproteinase by tissue plasminogen activator. Nat Med 9:1313-1317.
Wang Y F, Tsirka S E, Strickland S, Lipton S A, Tissue plasminogen activator knockout mice manifest smaller cerebral infarcts in a focal ischemia/reperfusion model (Abstract). Soc Neurosci Abstr 1996; 22:1694.
Wang Y F, Tsirka S E, Strickland S, Soriano S G, Lipton S A. Attenuated cerebral infarction in tissue plasminogen activator (tPA)-deficient mice (Abstract). Neurology 1997; 48:A408-A409.
Wang Y F, Tsirka S E, Strickland S, Stieg P E, Soriano S G, Lipton S A. Enhanced damage after tPA administration in the intravascular filament model of focal cerebral ischemia/reperfusion (Abstract). Soc Neurosci Abstr 1997 [Abstract]. Soc Neurosci Abstr 1997; 23:1921.
Wang Y F, Tsirka S E, Strickland S, Stieg P E, Soriano S G, Lipton S A. Tissue plasminogen activator (tPA) increases neuronal damage after focal cerebral ischemia in wild-type and tPA-deficient mice. Nature Medicine 1998; 4:228-231.
Wink, D. A., Kim, S., Coffin, D., Cook, J. C, Vodovotz, Y., Chistodoulou, D., Jourd'heuil, D., and Grisham, M. B., Detection of S-nitrosothiols by fluorometric and colorhnetric methods, Methods in Enzymology, 301, 201-211 (1999).
Yeh, M W, Kaul, M., Zheng J, Nottet HSLM, Thylin M, Gendelman H E, Lipton S A. Cytokine-stimulated, but not HIV-infected, human monocyte-derived macrophages produce neurotoxic levels of L-cysteine. J Immunol 2000; 164:4265-4270.
Yong V W, Power C, Forsyth P, Edwards D R (2001) Metalloproteinases in biology and pathology of the nervous system. Nat Rev Neurosci 2:502-511.
Yong, V. W., Krekoski, C. A., Forsyth, P. A., Bell, R., and Edwards, D. R., Matrix metalloproteinases and diseases of the CNS, Trends in Neurosciences, 21, 75-80 (1998).
Zhang J W, Gottschall P E (1997) Zymographic measurement of gelatinase activity in brain tissue after detergent extraction and affinity-support purification. J Neurosci Methods 76:15-20.
Zhao B-Q, Ikeda Y, Ihara H, Urano T, Fan W, Mikawa S, Suzuki Y, Kondo K, Sato K, Nagai N, Umemura K (2004) Essential role of endogenous tissue plasminogen activator through matrix metalloproteinase 9 induction and expression on heparin-produced cerebral hemorrhage after cerebral ischemia in mice. Blood 103:2610-2616.
Zurakowski D, Vorwerk C K, Gorla M, Kanellopoulos A J, Chaturvedi N, Grosskreutz C L, Lipton S A, Dreyer E B. Nitrate therapy may retard glaucomatous optic neuropathy, perhaps through modulation of glutamate receptors. Vision Res 1998; 38:1489-1494.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A method for treating a neurological disorder, an opthalmological disorder, or a combination thereof in a mammal inflicted with a neurological disorder, an opthalmological disorder, or a combination thereof, the method comprising administering to the mammal in need of such treatment an effective amount of a compound of formula (I): 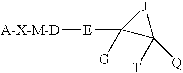
a metabolite thereof, a prodrug thereof, or a pharmaceutically acceptable salt thereof, wherein
A-X-M is a hydrophobic group;
D is O, S, (C₁-C₆)alkyl, a direct bond, SO₂, SO, C(═O)NR, C(═O)O, NRC(═O), or OC(═O);
E is a direct bond, (C₁-C₆)alkyl, (C₃-C₈)cycloalkyl, (C₂-C₆)alkenyl, or (C₂-C₆)alkynyl, wherein any alkyl, cycloalkyl, alkenyl, or alkynyl of E is optionally substituted with one or more (C₁-C₆)alkyl, hydroxy, (C₁-C₆)alkoxy, cyano, nitro, halo, SR, NRR, or COOR, wherein each R is independently H or (C₁-C₆)alkyl;
J is S or O;
G, T, and Q are each independently H, (C₁-C₆)alkyl, or cyano.

2. The method of claim 1 wherein A-X-M is a saturated or partially unsaturated hydrocarbon chain comprising one or more carbon atoms and optionally comprising one or more oxy (—O—), thio (—S—), sulfinyl (—SO—), sulfonyl (S(O)₂—), or NR_{f} in the chain, wherein each R_{f} is independently hydrogen or (C₁-C₆)alkyl;
wherein the saturated or partially unsaturated hydrocarbon chain is optionally substituted with one or more oxo (═O), hydroxy, cyano, halo, nitro, trifluoromethyl, trifluoromethoxy, (C₁-C₆)alkyl, (C₁-C₆)alkoxy, (C₁-C₆)alkoxy(C₁-C₆)alkyl, (C₃-C₈)cycloalkyl, aryl, heteroaryl, (C₃-C₈)cycloalkyl(C₁-C₆)alkyl, (aryl)(C₁-C₈)alkyl, (heteroaryl)(C₁-C₆)alkyl, (C₃-C₈)cycloalkyl oxy, (aryl)oxy, (heteroaryl)oxy, (C₃-C₈)cycloalkyl, (aryl)oxy(aryl), (heteroaryl)oxy(heteroaryl), (C₃-C₈)cycloalkyl oxy (C₁-C₆)alkyl, (aryl)oxy (C₁-C₆)alkyl, or (heteroaryl)oxy (C₁-C₆)alkyl; and
wherein any aryl, (C₃-C₈)cycloalkyl, or heteroaryl is optionally substituted with one or more oxo (═O), hydroxy, cyano, halo, nitro, trifluoromethyl, trifluoromethoxy, (C₁-C₆)alkyl, (C₁-C₆)alkoxy, (C₁-C₆)alkoxy(C₁-C₆)alkyl, (C₃-C₈)cycloalkyl, aryl, heteroaryl, (C₃-C₈)cycloalkyl(C₁-C₆)alkyl, (aryl)(C₁-C₈)alkyl, (heteroaryl)(C₁-C₆)alkyl, (C₃-C₈)cycloalkyl oxy, (aryl)oxy, (heteroaryl)oxy, (C₃-C₈)cycloalkyl, (aryl)oxy(aryl), (heteroaryl)oxy(heteroaryl), (C₃-C₈)cycloalkyl oxy (C₁-C₆)alkyl, (aryl)oxy (C₁-C₆)alkyl, or (heteroaryl)oxy (C₁-C₆)alkyl.

3. The method of claim 1 wherein A and M are each independently phenyl or monocyclic heteroaryl, wherein any phenyl or monocyclic heteroaryl is optionally substituted with one or more hydroxy, (C₁-C₆)alkyl, (C₁-C₆)alkanoyl, (C₁-C₆)alkanoyloxy, (C₁-C₆)alkoxy, cyano, nitro, halo, trifluoromethyl, trifluoromethoxy, SR, NRR, or COOR; and
X is O, S, SO, SO₂, C(═O)NR, C(═O)O, NRC(═O), OC(═O), NR, a direct bond, or (C₁-C₆)alkyl optionally substituted with one or more hydroxy, (C₁-C₆)alkoxy, cyano, nitro, halo, SR, NRR, or COOR.

4. The method of claim 1 wherein A-X-M is bicyclic aryl, bicyclic heteroaryl, or bicyclic alkyl; wherein any aryl, heteroaryl or alkyl is optionally substituted with one or more hydroxy, (C₁-C₆)alkyl, (C₁-C₆)alkanoyl, (C₁-C₆)alkanoyloxy, (C₁-C₆)alkoxy, cyano, nitro, halo, trifluoromethyl, trifluoromethoxy, SR, NRR, or COOR; wherein each R is independently H, (C₁-C₆)alkyl, phenyl, benzyl, or phenethyl.

5. The method of claim 1 wherein A-X-M is bicyclic aryl, bicyclic heteroaryl, or bicyclic alkyl.

6. The method of claim 1 wherein A-X-M is: 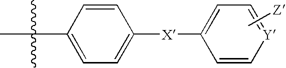
wherein
X′ is O, CH₂, or a direct bond;
Y′ is N or CH₂; and
Z′ is halo, OCH₃, or hydroxy.

7. The method of claim 1 wherein A-X-M is: 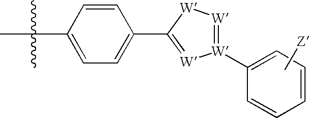
wherein
each W′ is independently N or CH; and
Z′ is halo, OCH₃, or hydroxy.

8. The method of claim 1 wherein A-X-M is: 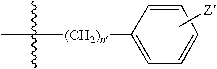
wherein
n′ is about 1 to about 4; and
Z′ is halo, OCH₃, or hydroxy.

9. The method of claim 1 wherein A-X-M is: 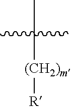
wherein
R′ is O, CH₂, or S; and
m′ is about 2 to about 7.

10. The method of claim 1 wherein A-X-M is: 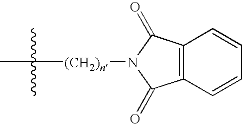
wherein
n′ is about 1 to about 4.

11. The method of claim 1 wherein A-X-M is: 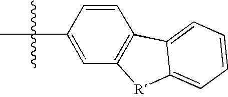
wherein
R′ is O, CH₂, or S.

12. The method of claim 1 wherein A-X-M is naphthyl.

13. The method of claim 1 wherein A is phenyl.

14. The method of claim 1 wherein M is phenyl.

15. The method of claim 1 wherein X is O.

16. The method of claim 1 wherein D is S, SO₂, or SO.

17. The method of claim 1 wherein D is SO₂.

18. The method of claim 1 wherein E is (C₁-C₆)alkyl.

19. The method of claim 1 wherein E is methyl.

20. The method of claim 1 wherein J is S.

21. The method of claim 1 wherein G is hydrogen.

22. The method of claim 1 wherein T is hydrogen.

23. The method of claim 1 wherein Q is hydrogen.

24. The method of claim 1 wherein A is phenyl, M is phenyl, X is O, D is SO₂, E is methyl, J is S, G is hydrogen, T is hydrogen, and Q is hydrogen.

25. The method of claim 1 further comprising administering a second neurological agent, or a pharmaceutically acceptable salt thereof.

26. The method of claim 1 wherein the mammal is a human.

27. The method of claim 1 wherein the neurological disorder, opthalmological disorder, or a combination thereof is an acute neurological disorder, opthalmological disorder, or a combination thereof.

28. The method of claim 1 wherein the neurological disorder, opthalmological disorder, or a combination thereof is a chronic neurological disorder, opthalmological disorder, or a combination thereof.

29. The method of claim 1 wherein the neurological disorder, opthalmological disorder, or a combination thereof arises from at least one of trauma, ischemic and hypoxic conditions.

30. The method of claim 1 wherein the neurological disorder, opthalmological disorder, or a combination thereof arises from at least one of painful neuropathy, neuropathic pain, diabetic neuropathy, drug dependence, drug withdrawal, drug addiction, depression, anxiety, movement disorders, tardive dyskinesia, cerebral infections that disrupt the blood-brain barrier, meningitis, meningoencephalitis, stroke, hypoglycemia, cerebral ischemia (stroke), cardiac arrest, spinal cord trauma, head trauma, perinatal hypoxia, cardiac arrest and hypoglycemic neuronal damage.

31. The method of claim 1 wherein the neurological disorder is a neurodegenerative disorder.

32. The method of claim 1 wherein the neurological disorder is a neurodegenerative disorder selected from the group of epilepsy, Alzheimer's disease, Huntington's disease, Parkinsonism, multiple sclerosis, and amyotrophic lateral sclerosis.

33. The method of claim 1 wherein the
neurological disorder, opthalmological disorder, or a combination thereof is glaucoma, retinal ischemia, ischemic optic neuropathy, macular degeneration, multiple sclerosis, sequalae of hyperhomocystinemia, convulsion, pain, depression, anxiety, schizophrenia, muscle spasm, migraine headache, urinary incontinence, drug withdrawal, nicotine withdrawal, opiate tolerance and withdrawal, emesis, brain edema, tardive dyskinesia, AIDS-induced dementia, ocular damage, retinopathy, a cognitive disorder, or a neuronal injury associated with HIV-infection.

34. The method of claim 1 wherein the neurological disorder, opthalmological disorder, or a combination thereof is a neuronal injury associated with HIV-infection selected from the group of dysfunction in cognition, movement and sensation.

35. A method for treating a neurological disorder, an opthalmological disorder, or a combination thereof in a mammal inflicted with a neurological disorder, opthalmological disorder, or a combination thereof, the method comprising administering to the mammal in need of such treatment an effective amount of a matrix metalloproteinase (MMP) inhibitor.

36. The method of claim 35 wherein the matrix metalloproteinase (MMP) is a gelatinase, collagenase, stromelysin, membrane-type MMP, MMP-23, MP-19, or matrilysin.

37. The method of claim 36 wherein the gelatinase is MMP-13, MMP-2 or MMP-9.

38.-48. (canceled)


SEQUENCE LISTING (5 sequences)

SEQ ID NO: 1 - protein, 8 aa, Artificial Sequence
  A synthetic peptide
CGVPDLGR

SEQ ID NO: 2 - protein, 7 aa, Artificial Sequence
  A synthetic peptide
CGVPDLR

SEQ ID NO: 3 - protein, 8 aa, Artificial Sequence
  A synthetic peptide
CGVPDVGK

SEQ ID NO: 4 - protein, 10 aa, Artificial Sequence
  A synthetic peptide
RPKPVENWRK

SEQ ID NO: 5 - protein, 8 aa, Artificial Sequence
  A synthetic peptide
PLGLAAAR